SEQUENCE LISTING (37 sequences)

SEQ ID NO: 1 - protein, 14 aa, Artificial Sequence
  chemokine-selective CXCR4 ectodomain-derived (poly)peptide first peptide
  VARIANT 1: any amino acid
  VARIANT 2: any amino acid
  VARIANT 3: any amino acid
  VARIANT 4: any amino acid
  VARIANT 5: any amino acid
  VARIANT 12: any amino acid
  VARIANT 13: any amino acid
  VARIANT 14: any amino acid
XXXXXWYFGNFXXX

SEQ ID NO: 2 - protein, 15 aa, Artificial Sequence
  chemokine-selective CXCR4 ectodomain-derived (poly)peptide second peptide
  VARIANT 1: any amino acid
  VARIANT 2: any amino acid
  VARIANT 3: any amino acid
  VARIANT 4: any amino acid
  VARIANT 5: any amino acid
  VARIANT 7: any amino acid
  VARIANT 10: any amino acid
  VARIANT 12: any amino acid
  VARIANT 15: any amino acid
XXXXXDXFYXNXLWX

SEQ ID NO: 3 - protein, 14 aa, Artificial Sequence
  ECL1
DAVANWYFGNFLCK

SEQ ID NO: 4 - protein, 15 aa, Artificial Sequence
  ECL2
DRYICDRFYPNDLWV

SEQ ID NO: 5 - protein, 9 aa, Artificial Sequence
  ECL1 positions 102 to 110
WYFGNFLCK

SEQ ID NO: 6 - protein, 9 aa, Artificial Sequence
  ECL2 positions 187 to 195
DRFYPNDLW

SEQ ID NO: 7 - protein, 21 aa, Artificial Sequence
  chemokine-selective CXCR4 ectodomain-derived (poly)peptide
WYFGNFLCKGGGDRFYPNDLW

SEQ ID NO: 8 - protein, 21 aa, Artificial Sequence
  chemokine-selective CXCR4 ectodomain-derived (poly)peptide
WYFGNFLCKDDDDRFYPNDLW

SEQ ID NO: 9 - protein, 21 aa, Artificial Sequence
  chemokine-selective CXCR4 ectodomain-derived (poly)peptide
WYFGNFLCKRRRDRFYPNDLW

SEQ ID NO: 10 - protein, 21 aa, Artificial Sequence
  chemokine-selective CXCR4 ectodomain-derived (poly)peptide
WYFGNFLCKKKKDRFYPNDLW

SEQ ID NO: 11 - protein, 114 aa, Homo sapiens
  human MIF 2-115
PMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 12 - protein, 31 aa, Homo sapiens
  human MIF 2-32
PMFIVNTNVPRASVPDGFLSELTQQLAQATG

SEQ ID NO: 13 - protein, 43 aa, Homo sapiens
  human MIF 38-80
IAVHVVPDQLMAFGGSSEPCALCSLHSIGKIGGAQNRSYSKLL

SEQ ID NO: 14 - protein, 32 aa, Homo sapiens
  human MIF 84-115
LAERLRISPDRVYINYYDMNAANVGWNNSTFA

SEQ ID NO: 15 - protein, 27 aa, Homo sapiens
  human MIF 54-80
SEPCALCSLHSIGKIGGAQNRSYSKLL

SEQ ID NO: 16 - protein, 15 aa, Homo sapiens
  human MIF 2-10
PMFIVNTNVPRASVP

SEQ ID NO: 17 - protein, 18 aa, Homo sapiens
  human MIF 6-23
VNTNVPRASVPDGFLSEL

SEQ ID NO: 18 - protein, 15 aa, Homo sapiens
  human MIF 13-27
ASVPDGFLSELTQQL

SEQ ID NO: 19 - protein, 15 aa, Homo sapiens
  human MIF 18-32
GFLSELTQQLAQATG

SEQ ID NO: 20 - protein, 16 aa, Homo sapiens
  human MIF 23-38
LTQQLAQATGKPPQYI

SEQ ID NO: 21 - protein, 16 aa, Homo sapiens
  human MIF 28-43
AQATGKPPQYIAVHVV

SEQ ID NO: 22 - protein, 21 aa, Homo sapiens
  human MIF 69-90
GGAQNRSYSKLLCGLLAERLR

SEQ ID NO: 23 - protein, 14 aa, Homo sapiens
  human MIF 76-90
YSKLLCGLLAERLR

SEQ ID NO: 24 - protein, 14 aa, Homo sapiens
  human MIF 81-94
CGLLAERLRISPDR

SEQ ID NO: 25 - protein, 14 aa, Homo sapiens
  human MIF 82-95
GLLAERLRISPDRV

SEQ ID NO: 26 - protein, 23 aa, Homo sapiens
  human MIF 38-60
IAVHVVPDQLMAFGGSSEPCALC

SEQ ID NO: 27 - protein, 27 aa, Homo sapiens
  human MIF 38-64
IAVHVVPDQLMAFGGSSEPCALCSLHS

SEQ ID NO: 28 - protein, 35 aa, Homo sapiens
  human MIF 38-72
IAVHVVPDQLMAFGGSSEPCALCSLHSIGKIGGAQ

SEQ ID NO: 29 - protein, 39 aa, Homo sapiens
  human MIF 38-76
IAVHVVPDQLMAFGGSSEPCALCSLHSIGKIGGAQNRSY

SEQ ID NO: 30 - protein, 11 aa, Homo sapiens
  human MIF 50-60
FGGSSEPCALC

SEQ ID NO: 31 - protein, 30 aa, Homo sapiens
  human MIF 50-80
FGGSSEPCALCSLHSIGKIGGAQNRSYSKL

SEQ ID NO: 32 - protein, 17 aa, Homo sapiens
  human MIF 51-67
GGSSEPCALCSLHSIGK

SEQ ID NO: 33 - protein, 27 aa, Homo sapiens
  human MIF 54-80
SEPCALCSLHSIGKIGGAQNRSYSKLL

SEQ ID NO: 34 - protein, 24 aa, Homo sapiens
  human MIF 57-80
CALCSLHSIGKIGGAQNRSYSKLL

SEQ ID NO: 35 - protein, 15 aa, Homo sapiens
  human MIF 60-74
CSLHSIGKIGGAQNR

SEQ ID NO: 36 - protein, 18 aa, Homo sapiens
  human MIF 62-80
LHSIGKIGGAQNRSYSKL

SEQ ID NO: 37 - protein, 114 aa, Mus musculus
  mouse MIF 2-115
PMFIVNTNVPRASVPEGFLSELTQQLAQATGKPAQYIAVHVVPDQLMTFSGTNDPCALCSLHSIGKIGGAQNRNYSKLLCGLLSDRLHISPDRVYINYYDMNAANVGWNGSTFA

(19) United States
(12) Patent Application Publication
(10) Pub. No.: US 2023/0242616 A1
(43) Pub. Date: Aug. 3, 2023

(54) CHEMOKINE-SELECTIVE CXCR4 ECTODOMAIN-DERIVED (POLY)PEPTIDE

(71) Applicants: Ludwig-Maximilians-Universität München, München (DE); Technische Universität München, München (DE)

(72) Inventors: Jürgen Bernhagen, München (DE); Omar El Bounkari, Diessen (DE); Aphrodite Kapurniotu, München (DE); Christos Kontos, Freising (DE)

(21) Appl. No.: 18/010,759

(22) PCT Filed: Jun. 15, 2021
(86) PCT No.: PCT/EP2021/066015

§ 371 Date: Dec. 15, 2022

(30) Foreign Application Priority Data
Jun. 16, 2020 (EP) 20180269.1

(51) Int. Cl. C07K 14/715; A61P 9/10; A61K 49/00

(52) U.S. Cl. CPC C07K 14/7158; A61P 9/10; A61K 49/0056

(57) ABSTRACT

The present invention relates to a chemokine-selective CXCR4 ectodomain-derived (poly)peptide comprising or consisting of a first peptide of (X1)(X2)(X3)(X4)(X5)WYFGNF(X6)(X7)(X8) (SEQ ID NO: 1) linked via a linker to a second peptide of (Y1)(Y2)(Y3)(Y4)(Y5)D(Y6)FY(Y7)N(Y8)LW(Y9) (SEQ ID NO: 2), wherein
(X1) is present or absent and, if present, is an amino acid, preferably D or A
(X2) is present or absent and, if present, is an amino acid, preferably A or G
(X3) is present or absent and, if present, is an amino acid, preferably V or A
(X4) is present or absent and, if present, is an amino acid, preferably A or G
(X5) is present or absent and, if present, is an amino acid, preferably N
(X6) is an amino acid, preferably L or A
(X7) is an amino acid, preferably C, A or S, more preferably C or A
(X8) is an amino acid, preferably K or A
(Y1) is present or absent and, if present, is an amino acid, preferably D or A
(Y2) is present or absent and, if present, is an amino acid, preferably R or A
(Y3) is present or absent and, if present, is an amino acid, preferably Y
(Y4) is present or absent and, if present, is an amino acid, preferably I or A
(Y5) is present or absent and, if present, is an amino acid, preferably C. A or S, more preferably C or A
(Y6) is present or absent and, if present, is an amino acid, preferably D, R or A
(Y7) is an amino acid, preferably P or A
(Y8) is an amino acid, preferably D or A
(Y9) is present or absent and, if present, is an amino acid, preferably V;
   and wherein said linker has a length of 0.2 to 5 nm, preferably 1 nm to 5 nm, more preferably 2 to 4 nm, and most preferably about 2.358 nm.

The present invention relates to a chemokine-selective CXCR4 ectodomain-derived (poly)peptide comprising or consisting of a first peptide of (X1)(X2)(X3)(X4)(X5)WYFGNF(X6)(X7)(X8) (SEQ ID NO: 1) linked via a linker to a second peptide of (Y1)(Y2)(Y3)(Y4)(Y5)D(Y6)FY(Y7)N(Y8)LW(Y9) (SEQ ID NO: 2), wherein

(X1) is present or absent and, if present, is an amino acid, preferably D or A

(X2) is present or absent and, if present, is an amino acid, preferably A or G

(X3) is present or absent and, if present, is an amino acid, preferably V or A

(X4) is present or absent and, if present, is an amino acid, preferably A or G

(X5) is present or absent and, if present, is an amino acid, preferably N

(X6) is an amino acid, preferably L or A

(X7) is an amino acid, preferably C, A or S, more preferably C or A,

(X8) is an amino acid, preferably K or A

(Y1) is present or absent and, if present, is an amino acid, preferably D or A

(Y2) is present or absent and, if present, is an amino acid, preferably R or A

(Y3) is present or absent and, if present, is an amino acid, preferably Y

(Y4) is present or absent and, if present, is an amino acid, preferably I or A

(Y5) is present or absent and, if present, is an amino acid, preferably C, A or S, more preferably C or A

(Y6) is present or absent and, if present, is an amino acid, preferably D, R or A

(Y7) is an amino acid, preferably P or A

(Y8) is an amino acid, preferably D or A

(Y9) is present or absent and, if present, is an amino acid, preferably V;

and wherein said linker has a length of 0.2 to 5 nm, preferably 1 nm to 5 nm, more preferably 2 to 4 nm, and most preferably about 2.358 nm.

In this specification, a number of documents including patent applications and manufacturer's manuals are cited. The disclosure or these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Chemokines are chemotactic cytokines that orchestrate cell trafficking and behavior in homeostasis and disease. Four chemokine sub-classes and their G protein-coupled receptor (GPCR)-type receptors (CKRs) constitute a complex ligand/receptor-network characterized by both specificity and redundancy^{1,2}. Chemokines are pivotal players in various inflammatory diseases including atherosclerosis^{1,3}. Therapeutic anti-cytokine approaches are successfully used in several inflammatory diseases and the positive results obtained with an interleukin-1β (IL-1β)-blocking antibody in the CANTOS trial have validated the inflammatory paradigm of atherosclerosis in humans and demonstrated the potential utility of anti-inflammatory drugs in patients with atherosclerotic disease. However, CANTOS also highlighted the need for molecular strategies with improved selectivity and less side effects⁴.

While anti-chemokine strategies such as antibodies or small molecule drugs (SMDs) have been established, targeting a specific chemokine/receptor axis remains challenging to due to the promiscuity in the chemokine network^{1,3,5,6}. In addition to antibodies and SMDs, soluble receptor-based approaches have proven as a powerful anti-cytokine strategy in inflammatory/immune diseases. For example, soluble tumor necrosis factor-receptor 1 (TNFR1)-based drugs are in clinical use for rheumatoid arthritis⁷. However, soluble receptor-based approaches based on GPCR ectodomains were sofar not established for chemokine receptors.

Hence, there is an unmet need for chemokine receptor-based poly(peptides) that are useful in therapy. This need is addressed by the present invention.

Accordingly, the present invention relates in first aspect to a chemokine-selective CXCR4 ectodomain-derived (poly)peptide comprising or consisting of a first peptide of

(X1)(X2)(X3)(X4)(X5)WYFGNF(X6)(X7)(X8) (SEQ ID NO: 1) linked via a linker to a second peptide of

(Y1)(Y2)(Y3)(Y4)(Y5)D(Y6)FY(Y7)N(Y8)LW(Y9) (SEQ ID NO: 2), wherein

(X1) is present or absent and, if present, is an amino acid, preferably D or A

(X2) is present or absent and, if present, is an amino acid, preferably A or G

(X3) is present or absent and, if present, is an amino acid, preferably V or A

(X4) is present or absent and, if present, is an amino acid, preferably A or G

(X5) is present or absent and, if present, is an amino acid, preferably N

(X6) is an amino acid, preferably L or A

(X7) is an amino acid, preferably C, S or A, more preferably C or A

(X8) is an amino acid, preferably K or A

(Y1) is present or absent and, if present, is an amino acid, preferably D or A

(Y2) is present or absent and, if present, is an amino acid, preferably R or A

(Y3) is present or absent and, if present, is an amino acid, preferably Y

(Y4) is present or absent and, if present, is an amino acid, preferably I or A

(Y5) is present or absent and, if present, is an amino acid, preferably C, S, or A, more preferably C or A

(Y6) is present or absent and, if present, is an amino acid, preferably D, R or A

(Y7) is an amino acid, preferably P or A

(Y8) is an amino acid, preferably D or A

(Y9) is present or absent and, if present, is an amino acid, preferably V;

and wherein said linker has a length of 0.2 to 5 nm, preferably 1 nm to 5 nm, more preferably 2 to 4 nm, and most preferably about 2.358 nm.

The term “comprise/comprising” is generally used in the sense of include/including, that is to say permitting the presence of one or more features or components. The terms “comprise” and “comprising” also encompass the more restricted terms “consist of” and “consisting of”.

As used in the specification and claims, the singular form “a”, “an” and “the” include plural references unless the context clearly dictates otherwise.

The term “(poly)peptide” in accordance with the present invention describes a group of molecules which comprises the group of peptides, consisting of up to 40 amino acids, as well as the group or polypeptides, consisting of more than 40 amino acids. Also encompassed by the term “(poly)peptide” are proteins as well as fragments of proteins. (Poly)peptides may further form dimers, trimers and higher oligomers, i.e. consisting of more than one (poly)peptide molecule. (Poly)peptide molecules forming such dimers, trimers etc. may be identical or non-identical. The corresponding higher-order structures are, consequently, termed homo- or heterodimers, homo- or heterotrimers etc. Homo- or heterodimers etc. also fall under the definition of the term “(poly)peptide”. The terms “polypeptide” and “protein” are used interchangeably herein and also refer to naturally modified polypeptides wherein the modification is effected e.g. by glycosylation, acetylation, phosphorylation and the like. Such modifications are well known in the art.

C-X-C chemokine receptor type 4 (CXCR4) also known as fusin or CD184 (cluster of differentiation 184) is a protein that in humans is encoded by the CXCR4 gene. CXCR-4 is an alpha-chemokine receptor initially thought to be specific for stromal ceN-derived-factor-1 (SDF-1 also called CXCL12), a molecule endowed with potent chemotactic activity for lymphocytes and other cell types. CXCR4 is one of two chemokine receptors that HIV can use to infect CD4+ T cells. CXCR4 belongs to the family of seven trans-membrane G-protein coupled receptors (GPCRs). Macrophage migration-inhibitory factor (MIF) is an alternate, non-cognate ligand of CXCR4 and further details on MIF and the MIF/CXCR4 interaction will be provided herein below.

The ectodomain or CXCR4 comprises three discontinuous loops, ECLs 1 to 3.

(X1)(X2)(X3)(X4)(X5)WYFGNF(X6)(X7)(X8) (SEQ ID NO: 1) is based on the sequence of ECL1 DAVANWYFGNFLCK (SEQ ID NO: 3) and (Y1)(Y2)(Y3)(Y4)(Y5)D(Y6)FY(Y7)N(Y8)LW(Y9) (SEQ ID NO: 2) is based on ECL2 DRYICDRFYPNDLWV (SEQ ID NO: 4). In the human CXCR4 ectodomain sequence, ECL1 can be found at amino acid positions 97 to 110 and ECL2 at amino acid positions 182 to 196.

In SEQ ID NO: 1 (X1), (X2), (X3), (X4) and (X5) may be present or absent. Similarly, in SEQ ID NO: 2 (Y1), (Y2). (Y3), (Y4), (Y5). (Y6), (Y7) and (Y9) may be present or absent. In the examples herein below fragments of ECL1 (amino acids 102 to 110) and ECL2 (amino acids 187 to 195) were tested and it was found that fragments lacking the indicated amino acids still retain 80% inhibitory capacity which is expressed as a binding affinity of well below 500 nM to macrophage migration-inhibitory factor (MIF) of all tested fragments.

It has also been tested in the examples to replace amino acids in ECL1 and ECL2. It has been found that in ECL1 DAVANWYFGNFLCK (SEQ ID NO: 3) and ECL2 DRYICDRFYPNDLWV (SEQ ID NO: 4) the underlined amino acids are essential and cannot be replaced or omitted without a significant loss of the inhibitory capacity. It has been found, for example, that in case one of the two underlined F in SEQ ID NO: 3 is replaced by A, the binding affinity to MIF is even completely abolished. On the other hand, it has been found that in ECL1 the amino acids in positions (X1), (X2), (X3), (X4), (X6), (X7) and (X8) and in ECL2 the amino acids in positions (Y1), (Y2), (Y4), (Y5), (Y6). (Y7) and (Y8) can be replaced by other amino acids without a significant loss of the inhibitory capacity. The replacement of the amino acids D97, D98, V99, L108, C109, K110, P191 and D193 by alanine even resulted in an improvement of the inhibitory capacity.

The term “amino acid” as used herein refers to an organic compound composed of amine (—NH₂) and carboxylic acid (—COOH) functional groups, generally along with a side-chain specific to each amino acid. The simplest amino acid glycine does not have a side chain (formula H₂NCH₂COOH). In amino acids that have a carbon chain attached to the α-carbon (such as lysine) the carbons are labeled in the order α, β, γ, δ, and so on. In some amino acids, the amine group may be attached, for instance, to the α-, β- or γ-carbon, and these are therefore referred to as α-, β- or γ-amino acids, respectively. All amino acids are in accordance with the present invention preferably α-amino acids (also designated 2-, or alpha-amino acids) which generally have the generic formula H₂NCHRCOOH, wherein R is an organic substituent being designated “side-chain”). In the simplest α-amino acid alanine (formula: H₂NCHCH₃COOH) the side is a methyl group.

Moreover, every amino acid (except glycine) can occur in two isomeric forms, because of the possibility of forming two different enantiomers (stereoisomers) around the central carbon atom. By convention, these are called L- and D-forms, analogous to left-handed and right-handed configurations. Generally only L-amino acids are manufactured in cells and incorporated into proteins. Thus, al amino acids are in accordance with the present invention preferably L-amino acids. Also in the appended examples section the used amino acids are L-amino acids, unless it is expressly stated that they are D-amino acids. More preferably, the amino acids are, in accordance with the present invention, L-α-amino acids.

As mentioned, the side-chain of an amino acid is an organic substituent, which, in the case of α-amino acids, is linked to the α-carbon atom. Hence, a side chain is a branch from the parent structure of the amino acid. Amino acids are usually classified by the properties of their side-chain. For example, the side-chain can make an amino acid a weak acid (e.g. amino acids D and E) or a weak base (e.g. amino acids K and R), and a hydrophile if the side-chain is polar (e.g. amino acids S and C) or a hydrophobe if it is non-polar (e.g. amino acids L and I). An aliphatic amino acid has a side chain being an aliphatic group. Aliphatic groups render the amino acid nonpolar and hydrophobic. The aliphatic group is preferably an unsubstituted branched or linear alkyl. Non-limiting examples of aliphatic amino acids are A, V, L, and I. In a cyclic amino acid one or more series of atoms in the side chain is/are connected to form a ring. Non-limiting examples of cyclic amino acids are P, F, W, Y and H. An aromatic amino acid is the preferred form of a cyclic amino acid, noting that in a cyclic amino acid a series of atoms in the side chain of the amino acid itself is connected to form a ring. In an aromatic amino acid the ring is an aromatic ring. In terms of the electronic nature of the molecule, aromaticity describes the way a conjugated ring of unsaturated bonds, lone pairs of electrons, or empty molecular orbitals exhibits a stronger stabilization than would be expected by the stabilization of conjugation alone. Aromaticity can be considered a manifestation of cyclic delocalization and of resonance. Non-limiting examples or aromatic amino acids are F, W, Y and H. A hydrophobic amino acid has a non-polar side chain making the amino acid hydrophobic. Non-limiting examples of hydrophobic amino acids are M, P, F, W, G, A, V, L and I. A polar, uncharged amino acid has a non-polar side chain with no charged residues. Non-limiting examples of polar, uncharged amino acids are S, T, N, Q, C, U (selenocysteine) and Y. A polar, charged amino acid has a non-polar side chain with at least one charged residue. Non-limiting examples of polar, charged amino acids are D, E, H, K and R.

The term “linker” as used in accordance with the present invention, preferably relates to peptide linkers, i.e. a sequence of amino acids, as well as to non-peptide linkers. An amino acid is generally defined as an organic compound that contains amine (—NH2) and carboxyl (—COOH) functional groups, along with a side chain (R group) specific to each amino acid. The amino acids may be naturally occurring amino acids, in particular the 21 amino acids being encoded by the genetic code. However, the amino acids may also be non-natural amino acids. For instance, 6-aminohexanoic acid as used in the appended examples is a derivative and analogue of the naturally occurring amino acid lysine. 12-aminododecanoic acid as also used in the appended examples and is an omega-amino fatty acid that is dodecanoic acid in which one of the terminal amino hydrogens has been replaced by an amino group. Hence, 6-aminohexanoic acid and 12-aminododecanoic acid are two examples of non-natural amino acids.

The term “non-peptide linker”, as used in accordance with the present invention, refers to linkage groups having two or more reactive groups but excluding peptide linkers. For example, the non-peptide linker may be a chemical compound having at least two reactive groups that link the molecules of SEQ ID NOs 1 and 2. Suitable reactive groups of chemical compounds include an aldehyde group, a propionic aldehyde group, a butyl aldehyde group, a maleimide group, a ketone group, a vinyl sulfone group, a thiol group, a hydrazide group, a carbonylimidazole group, an imidazolyl group, a nitrophenyl carbonate (NPC) group, a trysylate group, an isocyanate group, and succinimide derivatives. Examples of succinimide derivatives include succinimidyl propionate (SPA), succinimidyl butanoic acid (SBA), succinimidyl carboxymethylate (SCM), succinimidyl succinamide (SSA), succinimidyl succinate (SS), succinimidyl carbonate, and N-hydroxy succinimide (NHS). The at least two reactive groups may be the same or different and are preferably the same.

The “free” N-terminus (—NH₂) and/or the “free” C-terminus (—COOH) of the CXCR4 ectodomain-derived (poly)peptide according to the first aspect may be modified. For instance, the N-terminus may be acetylated and/or the C-terminus may be modified by amidation (—CONH₂).

In accordance with the first aspect of the invention the linker has a length of 0.2 to 5 nm, preferably 1 nm to 5 nm, more preferably 2 to 4 nm, and most preferably about 2.358 nm. The term “about” as used herein refers to “±20%”, preferably “±10%” and most preferably “±5”.

As can be taken from the appended examples, it has been surprisingly found that a chemokine-selective CXCR4 ectodomain-derived (poly)peptide according to the first aspect of the invention is capable of specifically inhibiting the interaction of CXCR4 and MIF but does not interfere or does not substantially interfere with the interaction of CXCR4 with CXCL12.

The term “does not substantially interfere with the interaction of CXCR4 with CXCL12” means that the chemokine-selective CXCR4 ectodomain-derived (poly)peptide according to the first aspect has a binding affinity to CXCL12 which is above 5 μM.

The chemokine-selective CXCR4 ectodomain-derived (poly)peptide according to the first aspect has a binding affinity to MIF which is with increasing preference below 1 μM, below 500 nM, below 250 nM, below 200 nM, below 100 nM and below 50 nM. The binding affinity is preferably determined by a fluorescence spectrometric binding assay as shown in the appended examples.

Macrophage migration-inhibitory factor (MIF) is an evolutionarily conserved, multi-functional inflammatory mediator that is structurally distinct from other cytokines^{8,9,10,11,12}. The MIF protein family also comprises D-dopachrome tautomerase (D-DT)/MIF-2¹³. MIF is an upstream regulator of the host innate and adaptive immune response, and, if dysregulated, is a key driver of acute and chronic inflammation, and cardiovascular diseases including atherosclerosis^{8,9,11,12,14,15,16,17,18}. Atherosclerotic vascular inflammation is orchestrated by chemokines, from leukocyte recruitment to foam cell formation and advanced plaque remodeling¹³. Examples are the classical chemokines CCL2 and CXCL1/CXCL8, but atypical chemokines (ACKs) that are structurally different from classical chemokines and yet interact with CKRs, have emerged as additional players in inflammation and atherogenesis^{16, 17}. Contrary to its eponymous name, MIF is recognized as a prominent ACK that enhances atherogenic leukocyte recruitment through non-cognate interactions with CXC motif chemokine receptors type 2 (CXCR2) and 4 (CXCR4)^{14,16,17}. Activation of CXCR4 by MIF thus contributes to the pathogenesis of atherosclerosis. It also contributes to cancer metastasis (Dessein at al., Cancer Res 2010; “Autocrine induction of invasive and metastatic phenotypes by the MIF-CXCR4 axis in drug-resistant human colon cancer cells”). Furthermore, MIF (and also MIF-2), are the sole ligands for the single-spanning type-II membrane protein CD74/invariant chain, through which they exert cardioprotective effects in the ischemic heart^{15,16,19,20}.

Besides other functions, the interaction of CXCR4 with CXCL12 mediates atheroprotection. It is therefore important to design a chemokine-selective CXCR4 ectodomain-derived (poly)peptide that specifically inhibits the above-described maleficial interaction of CXCR4 and MIF which at the same time does not interfere with the beneficial interaction of CXCR4 with CXCL12.

Based on the illustrative peptides ‘msR4Ms’ it is demonstrated in the examples that the (poly)peptides of the first aspect of the invention selectively bind MIF with nanomolar affinity and block the MIF/CXCR4 axis without affecting the CXCL12/CXCR4 interaction. MIF- but not CXCL12-elicited CXCR4 signaling, leukocyte chemotaxis, and foam cell formation is blocked. The achieved potency compares well with established, but pathway-non-selective MIF inhibitors, whereas the (poly)peptides of the first aspect do not interfere with the cardioprotective MIF/CD74 axis. Importantly, upon its in-vivo-administration a (poly)peptide of the first aspect localizes to atherosclerotic plaques, blocks leukocyte adhesion in atherosclerotic arteries, and potently inhibits atherosclerosis and vascular inflammation in hyperlipidemic Apoe−/− mice in vivo.

Finally, the selectivity of the (poly)peptides of the first aspect for MIF was confirmed in atherosclerotic plaques from human carotid-endarterectomy (CEA) specimens. In summary, by capitalizing on the distinctive nature of the CXCR4/MIF/CXCL12 network, an engineered CXCR4 ectodomain-based mimicry principle is provided that advantageously differentiates between disease-exacerbating (MIF) and protective pathways (CXCL12). The CXCL12/CXCR4 pathway exhibits critical homeostatic functions in resident arterial endothelial and smooth muscle cells and has a critical atheroprotective role^{26, 27}.

As discussed above, the (poly)peptide of the first aspect of the invention is based on the two discontinuous loops, ECL1 and ECL2, of the ectodomain of CXCR4. In the ectodomain of CXCR4 ECL1 and ECL2 are not adjacent to each other but are separated by more than 70 amino acids. It has been surprisingly found that ECL1 and ECL2 can be linked by a short linker having a length or 0.2 to 5 nm and the resulting (poly)peptide is still capable to specifically inhibit the interaction of CXCR4 and MIF. While it was known from Rajasekaran et al. (2016), The Journal of Biological Biochemistry, 291(30):15881-1895, that the domain of the ectodomain of CXCR4 which contributes to the binding to MIF is mainly located in the ECL1 and ECL2 area it was not obvious that the two loops alone are sufficient in order to ensure binding specificity to MIF while at the same time not interfering with the binding of CXCR4 to CXCL12. The data in Rajasekaran et al. (2018) teaches that also the receptor N-terminal domain of CXCR4 contributes to MIF binding, whereas it was surprisingly found in connection with the present invention that the receptor N-terminal domain is not essential for the binding specificity of CXCR4 to MIF.

It is also known that binding of MIF to CD74 involves MIF residues Pro-2, 80-87, and Tyr-100, while MIF binding to CXCR2 requires a pseudo-ELR motif, similar to CXCL8^{19, 21, 22, 23}. In contrast, the cognate CXCR4 ligand CXCL12 is an ELR-negative chemokine and the CXCL12/CXCR4 interface involves the receptor N-terminus and the RFFESH motif of CXCL12 at site 1 and the chemokine N-terminus and an intramembrane receptor groove at site 2. MIF binding to CXCR4 encompasses an extended N-like loop of MIF with contribution from Pro-2. Unlike for CXCL12, the MIF N-terminus around Pro-2 is rigid and likely unable to insert into the groove of CXCR4.

It was likewise not obvious that ECL1 and ECL2 can be connected by a short linker having a length of only 0.2 nm to 5 nm and that such a short linker is sufficient in order to put ECL1 and ECL2 into a three dimensional configuration which is capable of specifically inhibiting the interaction of CXCR4 and MIF. In this respect, it is of note that the inventors determined the distance of ECL1 and ECL2 in the crystals structure of the ectodomain of CXCR4 and found that the Lys 110 of ECL1 and the Asp 182 of ECL2 are about 2.25 nm apart from each other. For this reason, the linker of 0.2 nm to 5 nm in length is preferably 1 nm to 5 nm, more preferably 2 to 4 nm, and most preferably about 2.358 nm in length. It is technically advantageous to link ECL1 and ECL2 by a short linker as compared to the over 70 amino acids in nature since this results in a shorter (poly)peptide which is expected to penetrate tissue better in vivo and is less likely to exert adverse immune reactions when being administered to a subject. In addition, shorter (poly)peptides have an improved solubility and are much less costly to produce for pharmaceutical purposes.

It has also been tested in the examples to link the two cysteines or ECL1 DAVANWYFGNFLCK (SEQ ID NO: 3) and ECL2 DRYICDRFYPNDLWV (SEQ ID NO: 4) by a disulfide bond. It is of note that the disulfide bond does not interfere with inhibitory capacity of MIF binding. However, it was surprisingly found that the disulfide resulted in the undesired binding of the (poly)peptide to CXCL12. For this reason, it is preferred that no disulfide bonds are formed between the amino acid sequences of SEQ ID NOs 1 and 2 including the linker. As will be discussed herein below, disulfide bonds may be formed between cysteines being located outside the amino acid sequences of SEQ ID NOs 1 and 2 including the linker.

Signaling experiments, chemotaxis, foam cell formation, and leukocyte recruitment studies in the atherosclerotic vasculature in the appended examples demonstrate that the (poly)peptides of the invention can act as agonist-specific anti-atherogenic compounds, blocking CXCR4-mediated atherogenic MIF activities, while sparing CXCL12 and protective MIF/CD74-dependent signaling in cardiomyocytes. It is shown that the (poly)peptides of the invention not only home to and mark atherosclerotic plaque tissue in a MIF-specific manner in mouse and human lesions, but functionally protect from lesion development and atherosclerotic inflammation in an atherogenic Apoe model in vivo.

In accordance with a preferred embodiment of the first aspect of the invention, the linker comprises or consists of 1 to 8, and preferably 2 or 3 amino acids.

In accordance with this preferred embodiment, the linker is a peptide linker and the number of amino acids results in a linker length between 0.2 nm and 5 nm as required by the first aspect of the invention. The use of peptide linkers is exemplified in the appended examples, wherein ECL1- and ECL2-derived peptides were inter alia linked by the three amino acids GGG, DDD, RRR or KKK.

In accordance with another preferred embodiment of the first aspect of the invention, the linker comprises or consists of non-natural amino acids.

The use of non-natural amino acids (such as beta-alanine) is, for example, advantageous, since non-natural amino acids may display an improved stability against peptidases in vivo.

In accordance with a more preferred embodiment, the non-natural amino acids are selected from the group consisting of 6-aminohexanoic acid (6-Ahx), 12-amino-dodecanoic acid (12-Ado) and 3,6-dioxaoctanoic acid (O2Oc).

In accordance with a even more preferred embodiment wherein the linker comprises or consists of 6-Ahx-12-Ado or O2Oc-12-Ado, and preferably consists of 6-Ahx-12-Ado.

The amino acids 6-aminohexanoic acid (6-Ahx), 12-amino-dodecanoic acid (12-Ado) and 3,6-dioxaoctanoic acid (O2Oc) were used in the examples herein below to connect ECL1 and ECL2. It is in particular preferred to use the amino acids 6-Ahx and 12-Ado or the amino acids O2Oc and 12-Ado as two amino acids linking SEQ ID NOS 1 and 2. This is because this results in a linker length of about 2.358 nm which closely resembles the natural distance of ECL1 and ECL2 of 2.25 nm in the three dimensional folding structure of the CXCR4 ectodomain.

The use of 6-Ahx and 12-Ado is preferred over the use O2Oc and 12-Ado since the (poly)peptide with the latter linker O2Oc and 12-Ado displays a higher self-assembly propensity.

6-Ahx, 12-Ado and O2Oc are non-limiting but preferred examples of non-natural amino acids that may be used in the linker according to the invention.

It is also possible to use the non-natural amino acid 1,13-diamino-4,7,10-trioxatridecan-succinamic acid with a length of around 1.6 nm (Category A) together with one of 2-(2-(2-(2-amino)ethoxy)ethoxy)ethylamino)-diglycolic acid, 4-amino-benzoic acid and 12-amino-4,7,10-trioxa-dodecanoic acid, all having an approximate length of around 0.6 nm (Category B).

Also the same non-natural amino acid from category B may be used twice in a row, or twice in a row the non-natural amino acid 8-aminooctanoic acid (approx. length of 2.1 nm).

Yet further, O1Pen-C1Pen (=2×5-amino-3-oxapentanoic acid) or 4-times 5-amino-3-oxapentanoic acid (or O1Pen) may be used to reach a length of about 2.2 nm.

In addition, a linker providing for a distance of about 1 nm may be used. Examples are 1×8-aminooctanoic acid and 2×5-amino-3-oxapentanoic acid.

In accordance with another preferred embodiment of the first aspect of the invention the linker comprises or consists of three naturally-occurring amino acids, preferably selected from G, D, R and K, wherein the linker is most preferably selected from DDD and RRR.

As discussed herein above, the use of peptide linkers is exemplified in the appended examples, wherein ECL1- and ECL2-derived peptides were inter ala linked by the three amino acids GGG, DDD, RRR or KKK.

The linkers DDD and RRR are particularly preferred since they resulted in an improved solubility of the (poly)peptide as compared to the use of the linker of 6-Ahx-12-Ado.

In accordance with further preferred embodiment of the first aspect of the invention the (poly)peptide is a cyclic (poly)peptide.

As discussed herein above, linking the side chains of the two cysteines within ECL1 and ECL2 including the linker resulted in a loss of the specificity of the (poly)peptide to MIF. Hence, the cycle is to be formed by outside of SEQ ID NO: 1 and SEQ ID NO: 2 between a residue being located N-terminally of SEQ ID NO: 1 and a residue being located C-terminally of SEQ ID NO: 2.

Such a circle formation is expected to not interfere with the binding specificity of the (poly)peptide to MIF and the non-binding to CXCL12. Moreover, such circle formation is expected to increase the stability of the (poly)peptide in vivo since it protects the ends (poly)peptide from degradation by proteases.

In accordance with a more preferred embodiment of the first aspect of the invention the (poly)peptide contains two cysteines or homocysteines being linked by an S—S bond.

S—S bonds or disulfide bridges are formed between thiol groups in two cysteine or homocysteine residues. S—S bonds are am important component of the secondary and tertiary structure of natural proteins.

In accordance with an even more preferred embodiment of the first aspect of the invention the two cysteines or homocysteines are preferably located at the N-terminus of the first peptide and the C-terminus of the second peptide.

Cysteines or homocysteines being located at the N-terminus of the first peptide (SEQ ID NO: 1) and the C-terminus of the second peptide (SEQ ID NO: 1) allow to keep the small size of the (poly)peptide and conformational flexibility while at the same time allowing for the discussed stability advantages of a cyclic (poly)peptide.

In accordance with an even more preferred embodiment of the first aspect of the invention the (poly)peptide is fused to (i) a component modulating serum half-life, wherein the component modulating serum half-life is preferably Fc domain of an antibody, an albumin binding tag, albumin or polyethylene glycol, (e) a component increasing the solubility of the (poly)peptide, wherein the component is preferably selected from a peptide comprising acids with positively and negatively charged side chains, betaines, polyionic tags, cyclodextrins, glycosyl moieties, and conjugated nanoparticles, and/or (iii) a diagnostic label, preferably a chromogenic label, a fluorogenic label, or an isotope.

The fusion of biologicals to components increasing serum or blood half-life and/or solubility is a widely used approach in order to improve the pharmacokinetic properties of biologicals (for review Strohl (2015), Bio Drugs, 29(4): 215-239 and Bech et al. (2018). ACS Medical Chemistry Letters: 9:577-580).

Non-limiting but preferred examples of components increasing serum or blood half-life are albumin (preferably human serum albumin (HSA)), lipids, fatty acids, cholesterol-Hike albumin binders, small molecule albumin binders (e.g. an oxynotomodulin analog), transferrin (Tf), linear or branched-chain monomethoxy poly-ethylene glycol (PEG), the constant fragment (Fc) domain of a human immunoglobulin (IgG), non-structured polypeptides such as XTEN (i.e. a class of unstructured hydrophilic, biodegradable protein polymers designed to increase the half-lives of therapeutic peptides), homo-amino acid polymer (HAP: HAPylation), a proline-alanine-serine polymer (PAS: PASylation), an elastin-like peptide (ELP: ELPylation), a negatively charged, and highly sialylated peptide (e.g., carboxy-terminal peptide [CTP; of chorionic gonadotropin (CG) β-chain].

The component modulating serum half-life is preferably an albumin binding tag, albumin or polyethylene glycol (PEG).

Albumin is a natural transport protein with multiple ligand binding sites, cellular receptor engagement, and a long circulatory half-life due to interaction with the recycling neonatal Fc receptor. These properties make albumin suitable for half-life extension and targeted intracellular delivery of drugs attached by covalent conjugation, genetic fusions, association or ligand-mediated association. Instead of the direct fusion of albumin, also an albumin binding tag can be used as fusion partner. Albumins are commonly found in blood so that the albumin becomes bound to the albumin tag upon administration of the fusion construct carrying the albumin binding tag. The albumin is preferably human serum albumin. Human serum albumin (HSA) is a globular, ‘all α-helical’ protein present in the circulatory system; in fact, it is the most abundant of all plasma proteins (˜60%), with an average concentration of 50 grams per liter.

Another modification that can be made to improve the pharmacokinetics of peptide or biologic drugs is the conjugation of the drug to either linear or branched-chain polyethylene glycol (PEG), resulting in increases in the molecular mass and hydrodynamic radius, and a decrease in the rate of glomerular filtration by the kidney. PEG is a highly flexible, uncharged, mostly non-immunogenic, hydrophilic, non-biodegradable molecule, which generates a larger hydrodynamic radius than an equivalently sized protein.

Peptide-Fc fusion drugs, also known as peptibodies, are a category of biological therapeutics in which the Fc region of an antibody is genetically fused to a peptide of interest. Also the primary reason for fusion of a binding moiety with Fc is half-life extension. Many biologically active proteins and peptides have very short serum half-lives due to fast renal clearance, which limits their exposure in the target tissue and, consequently, their pharmacological effects. The Fc domain prolongs the serum half-life of Fc-fusion proteins due to pH-dependent binding to the neonatal Fc receptor (FcRn), which salvages the protein from being degraded in endosomes. As an additional benefit, the Fc portion of Fc-fusion proteins allows easier expression and protein A-affinity purification, which confers practical advantages in the development of antibody and Fc-fusion therapeutics.

The use of an albumin binding tag is advantageous as compared to the use of albumin since the tag has a smaller size and is more stable as compared to the protein albumin. Non-limiting but preferred examples of albumin binding tags are fatty acids and in particular palmitic acid and γ-glutamic acid, noting that palmitic acid is used in the examples. Upon the administration of the cyclic inhibitor of the invention linked to a fatty acid being capable to bind to albumin to a subject, the fatty acid binds to albumin. Thereby the small cyclic inhibitor is sterically shielded from rapid proteolytic degradation in the blood and protected from rapid renal filtration due to the relatively large size of the albumin (HSA=66 kDa).

As mentioned, albumin is preferably human serum albumin. Human serum albumin contains nine different fatty acid binding sites, which can bind free fatty acids as well as fatty acids linked to other molecules (Bech et al. (2018), ACS Medical Chemistry Letters; 9:577-580).

The fatty acid is preferably a fatty acid having between 10 carbon atoms (e.g. lauric acid or laurate) and 18 carbon atoms (e.g. oleic acid or oleate), and more preferably between 14 (e.g. myristic acid or myristate) and 18 carbon atoms, and most preferably 16 carbon atoms (e.g. palmitic acid or palmitate). Also fatty di-acids can be used, such as octadecanedioic acid.

The application of solubility-enhancement tags (SETs) has been highly effective in overcoming solubility and sample stability issues. Non-limiting but preferred examples of components increasing the solubility of the (poly)peptide of the first aspect of the invention are peptides comprising acids with positively and negatively charged side chains, betaines, polyionic tags, and cyclodextrins, glycosyl moieties, conjugated nanoparticles

A diagnostic label allows to detect the (poly)peptide according to the invention within a subject or within a sample obtained from the subject. Non-limiting but preferred examples of diagnostic labels are a chromogenic label, a fluorogenic label, or an isotope.

Chromogenic labels generally rely on chemical reactions triggered by enzymes conjugated with either the primary or secondary antibody. Peroxidases such as horseradish peroxidise (HRP) are a commonly used tool for such reactions.

The fluorescent label is preferably a component selected from Alexa Fluor, Cy dyes and Fluorescein. Non-limiting further examples of fluorescent proteins are green fluorescent protein (GFP), yellow fluorescent protein (YFP), red fluorescent protein (RFP), cyan fluorescent protein (CFP) and infrared fluorescent protein (IFP).

The isotope (or radionuclide) is preferably either selected from the group of gamma-emitting isotopes, more preferably ⁹⁹mTc, ¹²³I, or ¹¹¹In, and/or from the group of positron emitters, more preferably ¹⁸F, ⁶⁴Cu, ⁶⁸Ga, ⁸⁶Y, ⁸⁹Zr, or ¹²⁴I and/or from the group of beta-emitters, more preferably ¹³¹I, ⁹⁰Y, ¹⁷⁷Lu, or ⁶⁷Cu, or from the group of alpha-emitter, preferably ²¹³Bi, or ²¹¹At. The radionuclide is more preferably a positron emitter since they are particularly suitable for diagnostics, e.g. via positron emission tomography imaging. In connection with the isotope, positron-emission tomography (PET) may be used. PET is a nuclear medicine functional imaging technique that is used to observe metabolic processes in the body as an aid to the diagnosis of diseases. PET detects pairs of gamma rays emitted indirectly by a positron-emitting radioligand, such as ¹⁸F, which is introduced into the body on a biologically active molecule called a radioactive tracer.

In accordance with a preferred embodiment of the first aspect of the invention, the (poly)peptide comprises a first peptide differing by no more than three, preferably by no more than two, more preferably by one amino acid mutation and most preferably by zero amino acid mutations from the peptide of DAVANWYFGNFLCK (SEQ ID NO: 3) and/or comprises a second peptide differing by no more than three, preferably by no more than two, more preferably by one amino acid mutation and most preferably by zero amino acid mutations from the peptide of DRYICDRFYPNDLVW (SEQ ID NO: 4).

In accordance with another preferred embodiment of the first aspect of the invention, the (poly)peptide comprises a first peptide differing by no more than three, preferably by no more than two, more preferably by one amino acid mutation and most preferably by zero amino acid mutations from the peptide of WYFGNFLCK (SEQ ID NO: 5) and/or comprises a second peptide differing by no more than three, preferably by no more than two, more preferably by one amino acid mutation and most preferably by zero amino acid mutations from the peptide of DRFYPNDLW (SEQ ID NO: 6).

Hence, also described herein is a chemokine-selective CXCR4 ectodomain-derived (poly)peptide comprising or consisting of a first peptide differing by no more than three, preferably by no more than two, more preferably by one amino acid mutation and most preferably by zero amino acid mutations from the peptide of DAVANWYFGNFLCK (SEQ ID NO: 3) linked via a linker to a second peptide differing by no more than three, preferably by no more than two, more preferably by one amino acid mutation and most preferably by zero amino acid mutations from the peptide of DRYICDRFYPNDLWV (SEQ ID NO: 4), wherein said linker has a length of 0.2 to 5 nm, preferably 1 nm to 5 nm, more preferably 2 to 4 nm, and most preferably about 2.358 nm. The linker is preferably as defined herein above in connection with the first aspect of the invention.

Furthermore described herein is a chemokine-selective CXCR4 ectodomain-derived (poly)peptide comprising or consisting of a first peptide differing by no more than three, preferably by no more than two, more preferably by one amino acid mutation and most preferably by zero amino acid mutations from the peptide of WYFGNFLCK (SEQ ID NO: 5) finked via a linker to a second peptide differing by no more than three, preferably by no more than two, more preferably by one amino acid mutation and most preferably by zero amino acid mutations from the peptide of DRFYPNDLW (SEQ ID NO: 6), wherein said linker has a length of 0.2 to 5 nm, preferably 1 nm to 5 nm, more preferably 2 to 4 nm, and most preferably about 2.358 nm. The linker is preferably as defined herein above in connection with the first aspect of the invention.

The “differing” amino acids may be deleted, added or substituted amino acids and are preferably substituted amino acids.

As discussed herein above, SEQ ID NOs 3 and 4 are the sequences of the loops ECL1 (positions 97 to 110) and ECL2 (positions 182 to 196) of the ectodomain of CXCR4. As is demonstrated in the examples, not all amino acids within the loops are important for the capability of the (poly)peptide for inhibiting the binding of MIF and CXCR4. It is in particular demonstrated that the loops can be shortened by the deletion of amino acids. Instead of the full-length ECL1 positions 102 to 110 (SEQ ID NO: 5) can be used and instead of the full-length ECL2 positions 187 to 195 (SEQ ID NO: 6) can be used.

In accordance with a further preferred embodiment of the first aspect of the invention, the (poly)peptide comprises or consists of an amino acid sequence selected form the group consisting of DAVANWYFGNFLCK-6-Ahx-12-Ado-DRYICDRFYPNDLWV; DAVANWYFGNFLCK-O2Oc-12-Ado-DRYICDRFYPNDLWV, WYFGNFLCK-6-Ahx-12-Ado-DRFYPNDLW, WYFGNFLCK-8-Aoc-DRFYPNDLW, WYFGNFLCK-Pen-01-Pen-DRFYPNDLW, WYFGNFLCK-GGG-DRFYPNDLW (SEQ ID NO: 7), WYFGNFLCK-DDD-DRFYPNDLW (SEQ ID NO: 8), WYFGNFLCK-RRR-DRFYPNDLW (SEQ ID NO: 9) and WYFGNFLCK-KKK-DRFYPNDLW (SEQ ID NO: 10).

The above (poly)peptides have been manufactured and their affinity to MIF has been tested in the appended examples of the application. All these (poly)peptides have a high affinity to MIF in the nM range.

The present invention relates in a second aspect to a composition, preferably a pharmaceutical composition comprising the (poly)peptide of the first aspect of the invention.

The definitions and preferred embodiments set forth herein above, where applicable, equally apply to the second aspect of the invention.

A composition is an article of manufacture comprising at least two components, wherein one component is the (poly)peptide of the first aspect of the invention. The other component may be, for example, an adjuvant or excipient, such as a solvent (e.g. water).

In accordance with the present invention, the term “pharmaceutical composition” relates to a composition for administration to a patient, preferably a human patient. The pharmaceutical composition of the invention comprises at least one the (poly)peptide of the first aspect of the invention. It may, optionally, comprise further molecules capable of altering the characteristics of the (poly)peptide of the first aspect of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s).

These pharmaceutical compositions can be administered to the subject at a suitable dose. The dosage regimen will be determined by the attending physician and clinical factors. As it is well known in the medical arts, dosages for any one patient depend upon many factors, including the patient's weight, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 0.2 to 60 μM of the (poly)peptide of the first aspect of the invention per day or less frequently, such as every two days, twice per week or ones per week.

The length of treatment needed to observe changes and the interval following treatment for responses to occur vary depending on the desired effect. The particular amounts may be determined by conventional tests, which are well known to the person skilled in the art.

Pharmaceutical compositions of the invention preferably will typically comprise a pharmaceutically acceptable carrier or excipient. By “pharmaceutically acceptable carrier or excipient” is meant a non-toxic solid, semisolid or liquid filler, diluent, encapsulating material or formulation auxiliary of any type (see also Handbook of Pharmaceutical Excipients 6ed. 2010, Published by the Pharmaceutical Press). Examples of suitable pharmaceutical carriers and excipients are well known in the art and include phosphate buffered saline solutions, water, emulsions, such as oil/water emulsions, various types of wetting agents, sterile solutions, organic solvents including DMSO etc. Compositions comprising such carriers or excipients can be formulated by well known conventional methods. The pharmaceutical composition may be administered, for example, orally, parenterally, such as subcutaneously, intravenously, intramusculary, intraperitonealy, intrathecally, transdermally, transmucosally, subdurally, locally or topically via iontopheresis, sublingually, by inhalation spray, aerosol or rectally and the like in dosage unit formulations.

The present invention relates in a third aspect to the (poly)peptide of the first aspect of the invention for use in the treatment or prevention of disease.

The definitions and preferred embodiments set forth herein above, where applicable, equally apply to the third aspect of the invention.

Also described herein is a method of treating or preventing a disease in a subject, comprising administering a therapeutically effective amount of the (poly)peptide of the first aspect of the invention to the subject.

Administering, as it applies in the present invention, refers to contact of the (poly)peptide of the first aspect of the invention with the subject to be treated, being preferably a human. A therapeutically effective amount of the (poly)peptide, when administered to a human or animal organism, is an amount of the (poly)peptide that induces the detectable pharmacologic and/or physiologic effect of inhibiting the binding between MIF and CXCR4.

In accordance with a preferred embodiment of the third aspect of the invention, the disease is an atherosclerotic disease, an inflammatory disease, a tumor, a neuroinflammatory or neuro-degenerative disease, or an autoimmune disease.

In accordance with a more preferred embodiment of the third aspect of the invention, the atherosclerotic disease, is an atherosclerotic disease in individuals with a high-MIF expression genotype as defined by the CATT6-8 or CATT-non-5/5 promoter polymorphism; and/or wherein the tumor is cancer, preferably metastatic cancer.

As discussed herein above, the binding of MIF to CXCR4 is inter aka involved in the pathogenesis of atherosclerosis. In more detail, the activation of the MIF and CXCR4 axes promotes leukocyte recruitment, preferably monocyte or lymphocyte recruitment, which mediates the exacerbating role of MIF in atherosclerosis and contributes to the wealth of other MIF biological activities.

Moreover, MIF is a pleiotropic inflammatory cytokine and a critical upstream mediator of innate immunity. Dysregulated MIF activity exacerbates autoimmune and inflammatory conditions, not only including atherogenesis but also septic shock, inflammatory lung diseases including acute respiratory distress syndrome (ARDS), autoimmune diseases, and cancer (Deepa Rajasekaran (2016), J Biol Chem.; 291(30):15881-15895; Calandra & Roger, Nat Rev Immunol 2003; October; 3(10):791-800; Morand et al., Nat Rev Drug Discov 2006, May; 5(5):399-410: Zemecke et al. Circulation 2008. March 25:117(12):1594-602; Sinitski et al., Thrombosis & Haemostasis 2019; April; 119(4):553-566; Kang & Bucala, Nat Rev Rheumatol 2019: July; 15(7):427-437).

Hence, the (poly)peptide of the first aspect of the invention is suitable for the treatment or prevention of various diseases, in particular the diseases recited in the above preferred embodiment and more preferred embodiment of the third aspect of the invention.

The (CATT)5-8 or (CATT)-non-5/5 polymorphism in the promoter region of the MIF gene is related to the progression of atherosclerosis (Valdés-Alvarado et al. 2014, J Immunol Res. 2014; 2014: 704854). Unless otherwise defined, all technical and scientific terms used herein have the same meaning as commonly understood by one of ordinary skill in the art to which this invention belongs. In case of conflict, the patent specification including definitions, will prevail.

All amino acid sequences provided herein are presented starting with the most N-terminal residue and ending with the most C-terminal residue (N-+C), as customarily done in the art, and the one-letter or three-letter code abbreviations as used to identify amino acids throughout the present invention correspond to those commonly used for amino acids.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I: B, E, G; B, E, H; B, E, I; B, F, G; B, F, H; B, F, I: C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show.

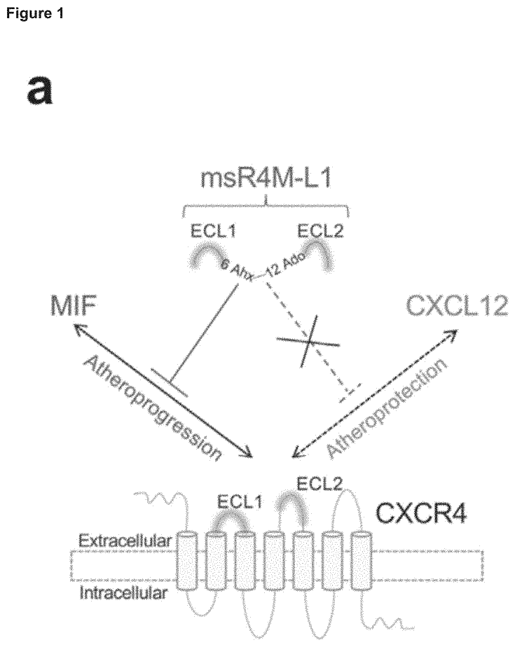

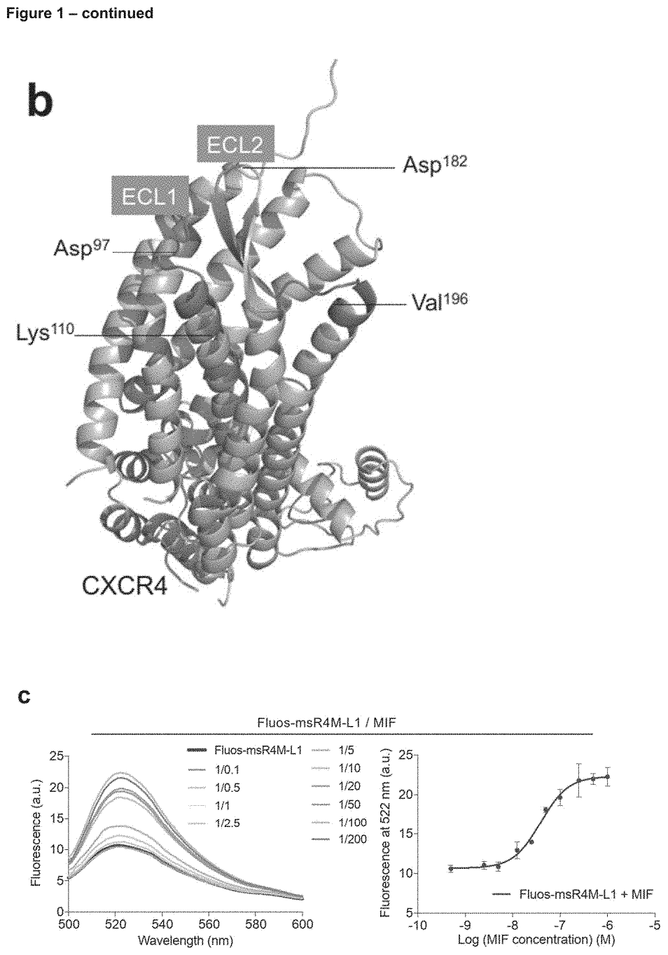

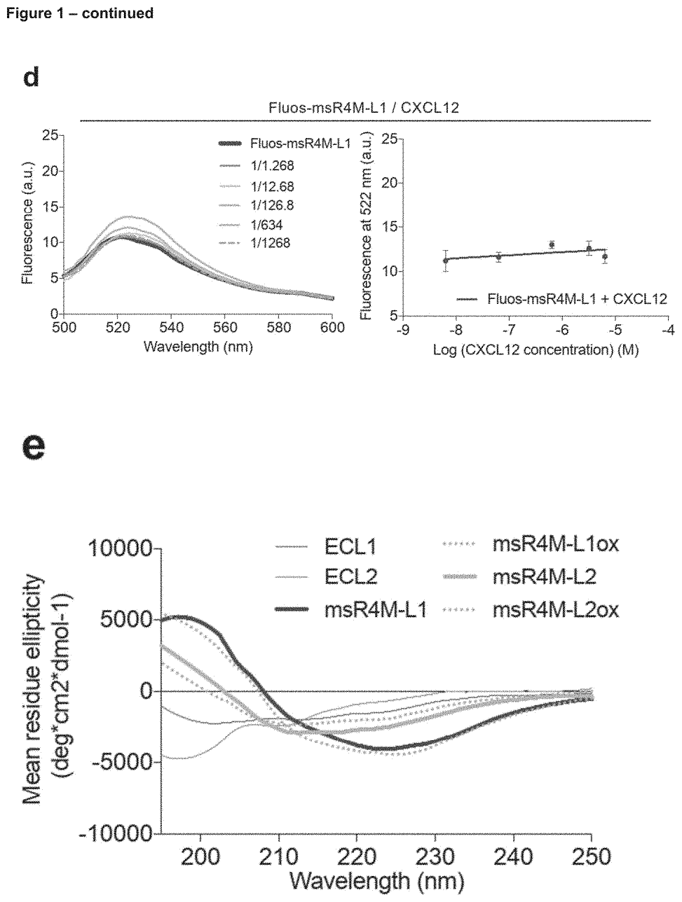

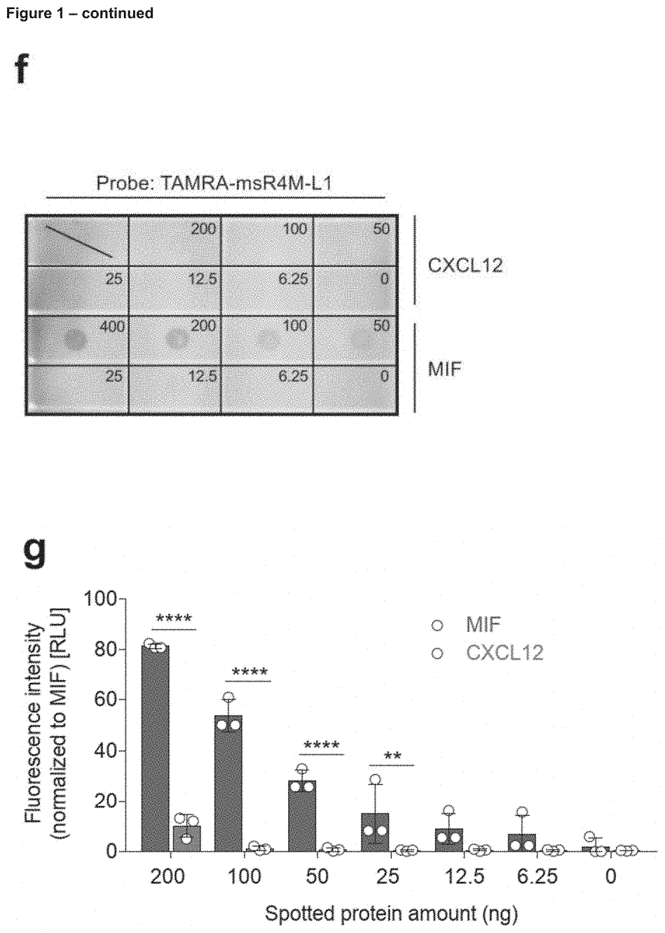

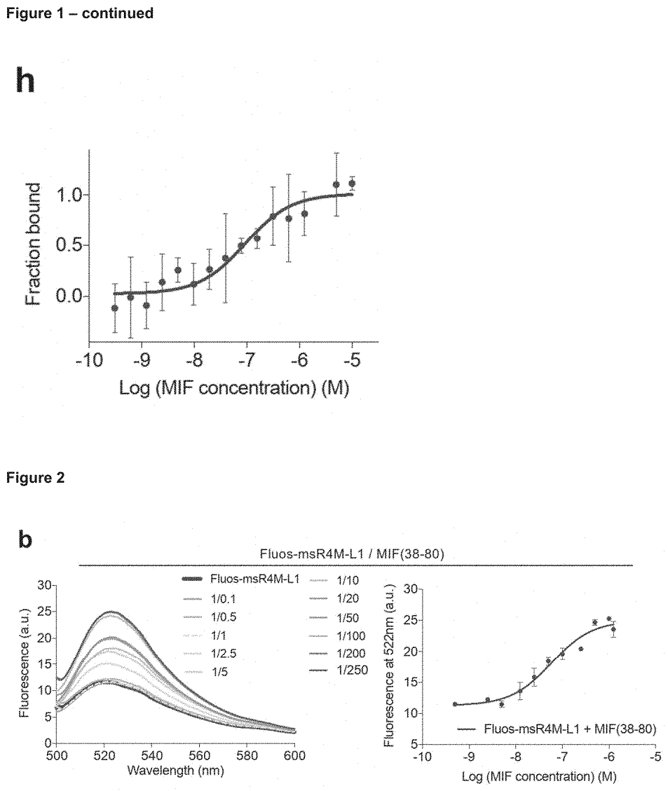

FIG. 1 The CXCR4 ectodomain mimic msR4M-L1 selectively binds to MIF but not CXCL12. a Schematic summarizing the design strategy to utilize extracellular loop moieties of CXCR4 to engineer a soluble mimic that binds MIF but not CXCL12. b Ribbon structure of human CXCR4 based on the crystal structure according to PDB code 4RWS³⁰. Sequences of extracellular loops ECL1 and ECL2 that were found to interact with MIF according to peptide array mapping²⁵ are highlighted in blue, and the N- and C-terminal residues of the ECL1 and 2 peptides are indicated. c Nanomolar affinity binding of msR4M-L1 to MIF as determined by fluorescence spectroscopic titrations. Emission spectra of Fluos-msR4M-L1 alone (blue) and with increasing concentrations of MIF at indicated ratios are shown (left panel; representative titration); binding curve derived from the fluorescence emission at 522 nm (right panel). d msR4M-L1 does not bind to CXCL12. Same as c but titration performed with increasing concentrations of CXCL12. e Conformation of CXCR4 ectodomain mimics as determined by far-UV CD spectroscopy. Mean residue ellipticity plotted over the wavelength between 195 and 250 nm. f-g Binding analysis between TAMRA-msR4M-L1 and MIF versus CXCL12 as determined by dot blot titration. f Representative blot; g quantification from three independent blots according to f. h Binding of msR4M-L1 to MIF as determined by microscale thermophoresis (MST). The fraction of MIF bound to TAMRA-msR4M-L1 is plotted against increasing concentrations of MIF. Data in d, g, and h are reported as means±SD from three independent experiments. Statistical analysis (g) was performed with unpaired T-test (*P<0.01, **P<0.001).

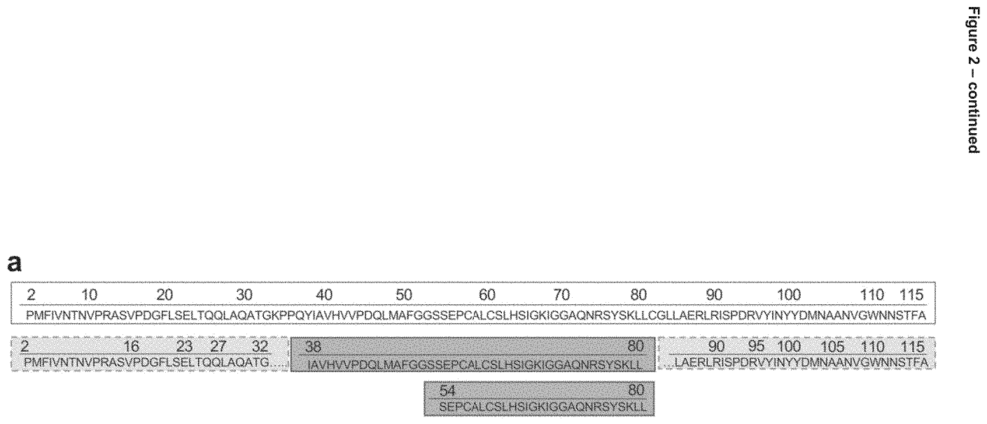

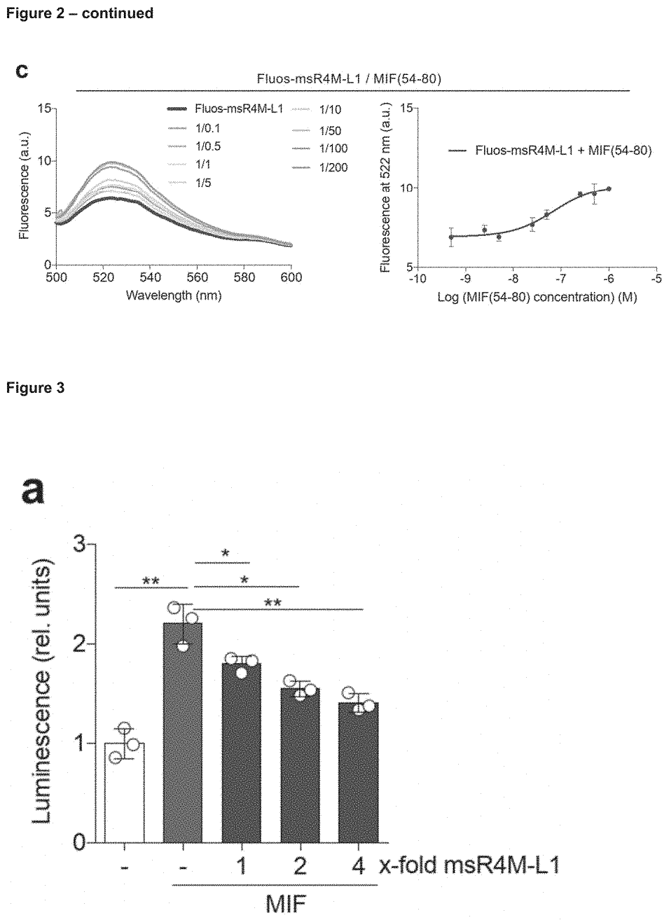

FIG. 2 Engineered CXCR4 mimic binds to a core region in MIF. a Amino acid sequence of human MIF (boxed, top). The msR4M-L1 binding core region of MIF (sequence 38-80 and 54-80) is indicated in blue, while non-binding stretches are in grey (bottom). b-c Nanomolar affinity binding of msR4M-L1 to MIF(38-80) (b) and MIF(54-80) (c) as determined by fluorescence spectroscopic titrations. Emission spectra of Fluos-msR4M-L1 alone (blue; 5 nM) and with increasing concentrations of MIF(38-80) (b) and MIF(54-80) (c) (left panels; representative titrations); binding curves derived from the fluorescence emission at 522 nm (right panels). Data in right panels are means±SD from three independent experiments.

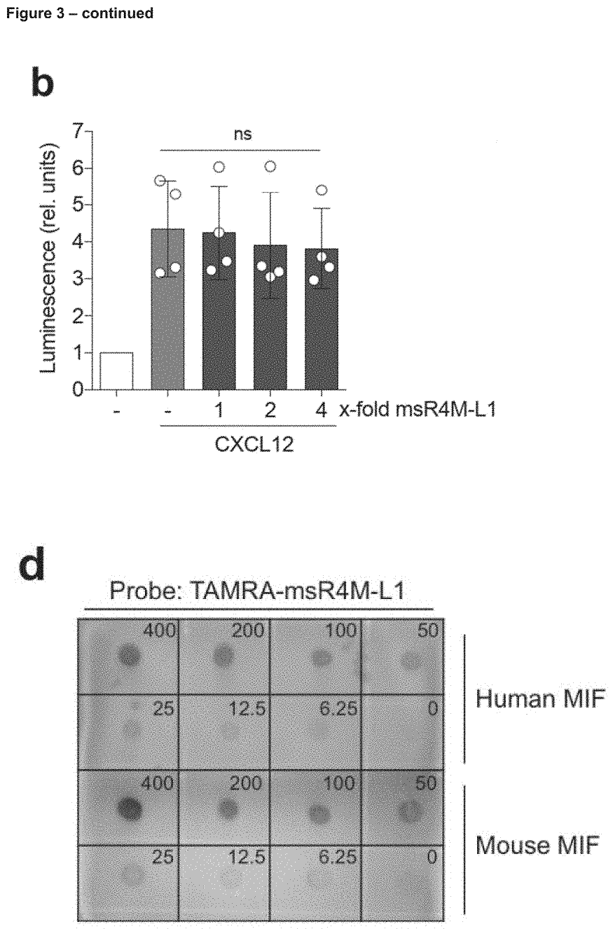

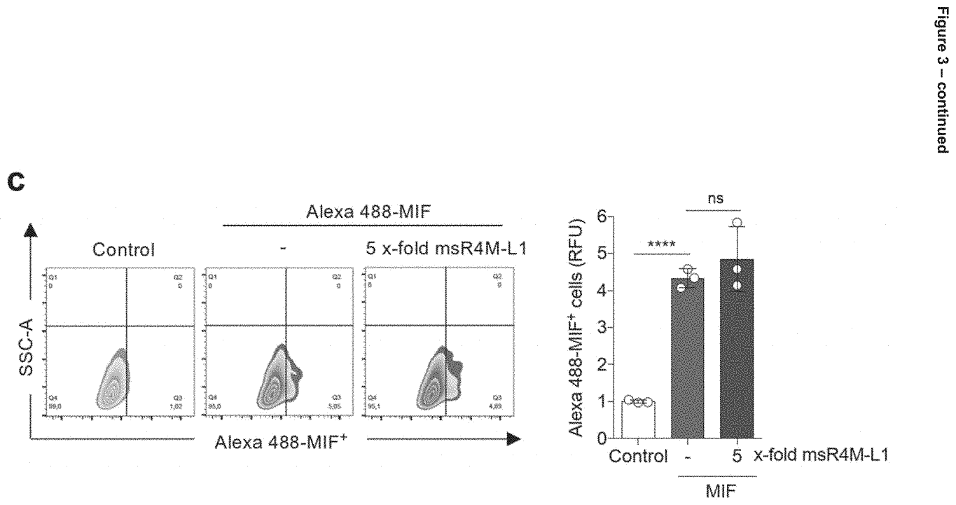

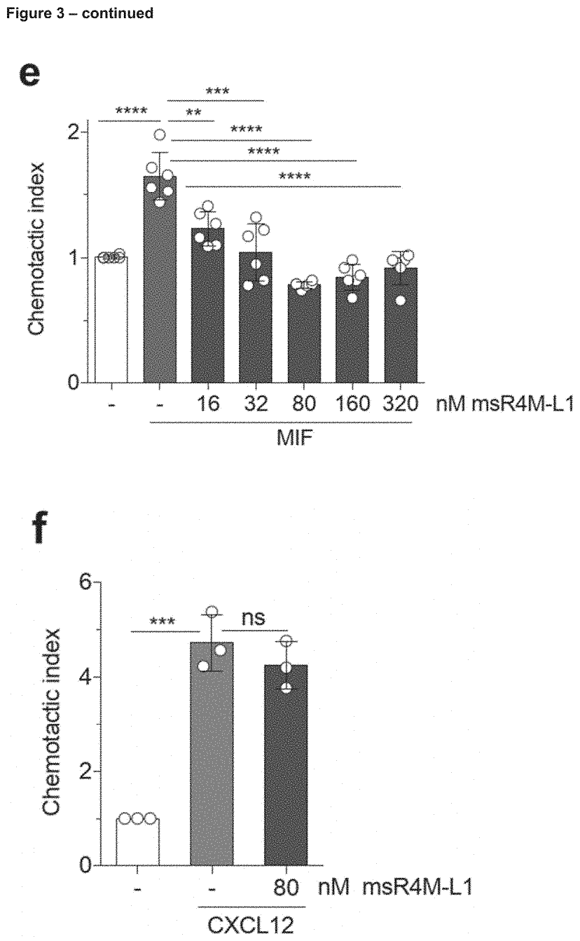

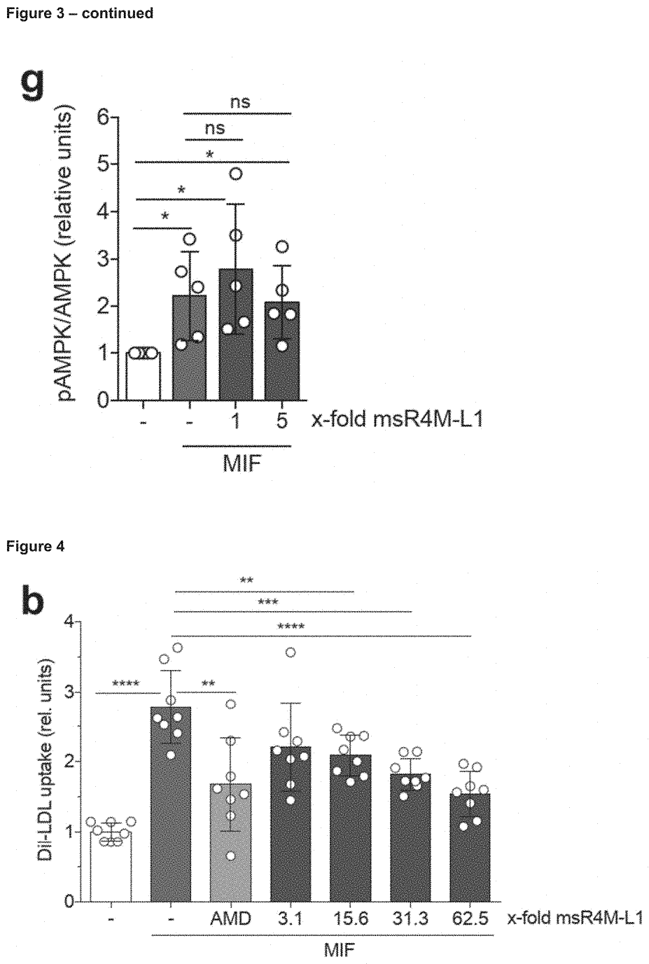

FIG. 3 CXCR4 ectodomain mimic msR4M-L1 selectively inhibits MIF-triggered cell surface CXCR4 binding, signaling and chemotaxis, but does not interfere with MIF-mediated AMPK signaling in cardiomyocytes. a-b MIF (a) but not CXCL12 (b) binding to and signaling through human CXCR4 in an S. cerevisiae system is attenuated by msR4M-L1 in a concentration-dependent manner. The molar excess of competing msR4M-L1 over MIF or CXCL12 is indicated. CXCR4 binding/signaling is read out by LacZ reporter-driven luminescence. c A 5-fold molar excess of msR4M-L1 does not interfere with binding of Alexa 488-MIF to CD74 expressed on HEK293-CD74 transfectants as measured by flow cytometry. left Shift of CD74 transfectants following Alexa 488-MIF binding (control indicates background); right quantification of three independent experiments. d Dot blot demonstrates that msR4M-L1 binds equally well to human and mouse MIF. Different protein amounts of human and mouse MIF were spotted as indicated and blots developed with TAMRA-labeled msR4M-L1. The blot shown is representative of three independently performed experiments. e-f Chemotactic migration (Transwell) of primary mouse spleen B lymphocytes elicited by 16 nM MIF (a) or CXCL12 (f) as chemoattractant and inhibitory effect of msR4M-L1. msR4M-L1 dose-dependently inhibits MIF-mediated chemotaxis (e), but the optimal inhibitory dose of 80 nM does not affect CXCL12-elicited chemotaxis (f). g msR4M-L1 does not interfere with MIF-triggered AMPK signaling in the human cardiomyocyte cell line HCM. MIF was applied at a concentration of 16 nM; msR4M-L1 added at 1- and 5-fold excess over MIF. AMPK signaling was measured using Western blot of HCM lysates developed against pAMPK and total AMPK. The densitometric ratio of pAMPK/AMPK indicates signaling intensity. Data are reported as means±SD of (n=3 (a); n=4 (b): n=3 (c); n=3-7 (a-f); and n=5 (g) independent experiments. Statistical analysis was performed with unpaired T-test (*P<0.05. **P<0.01, ***P<0.005, ****P<0.001).

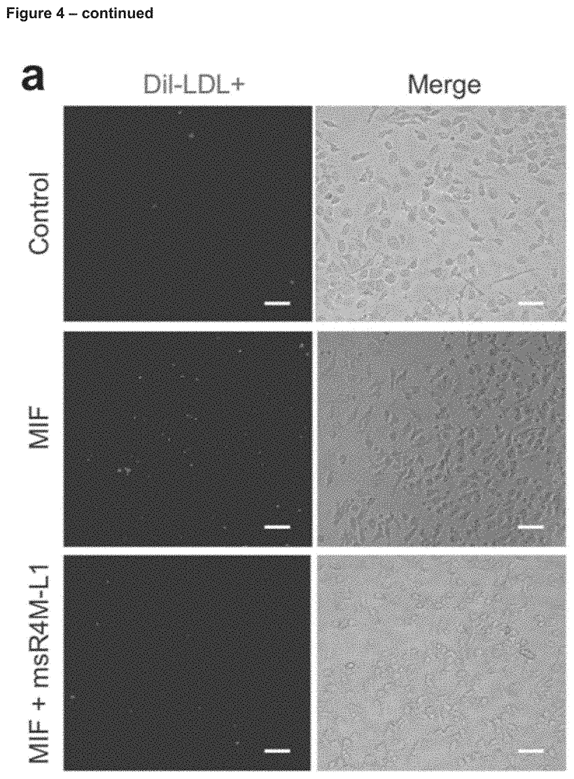

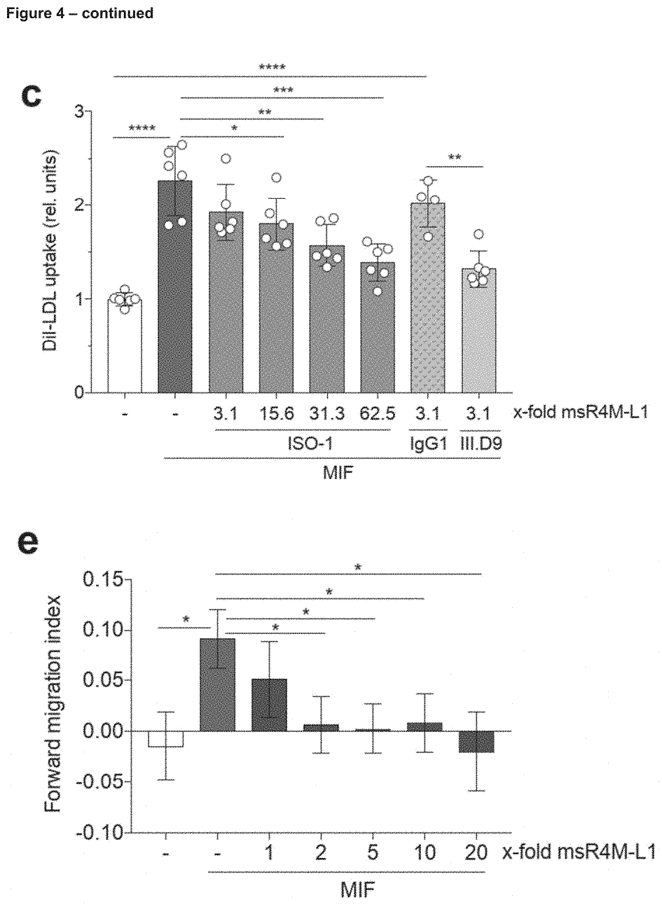

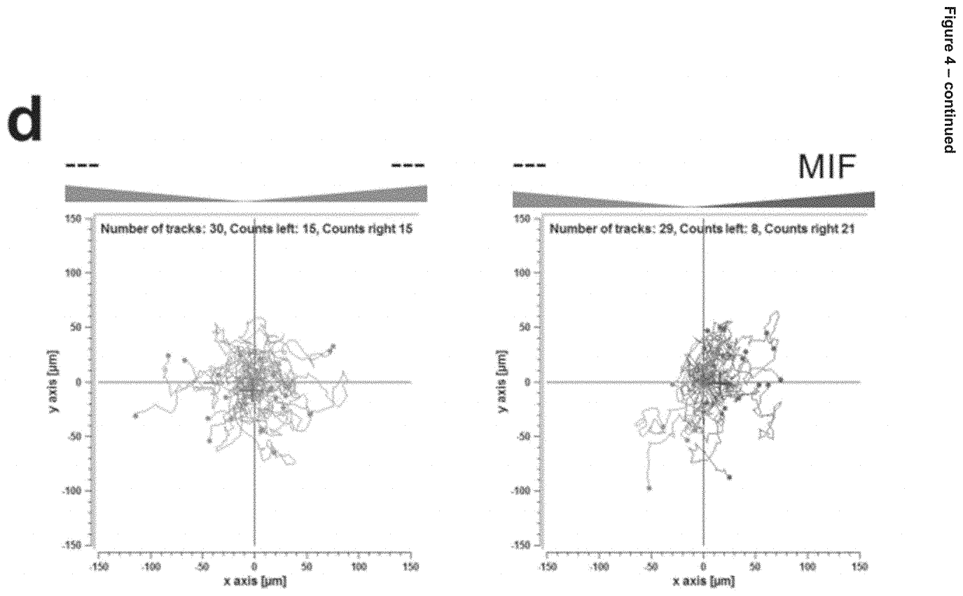

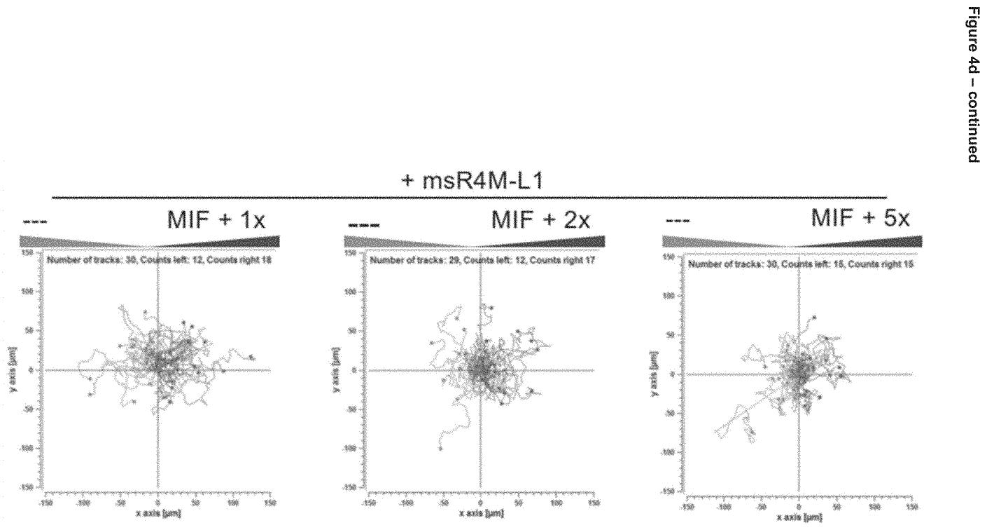

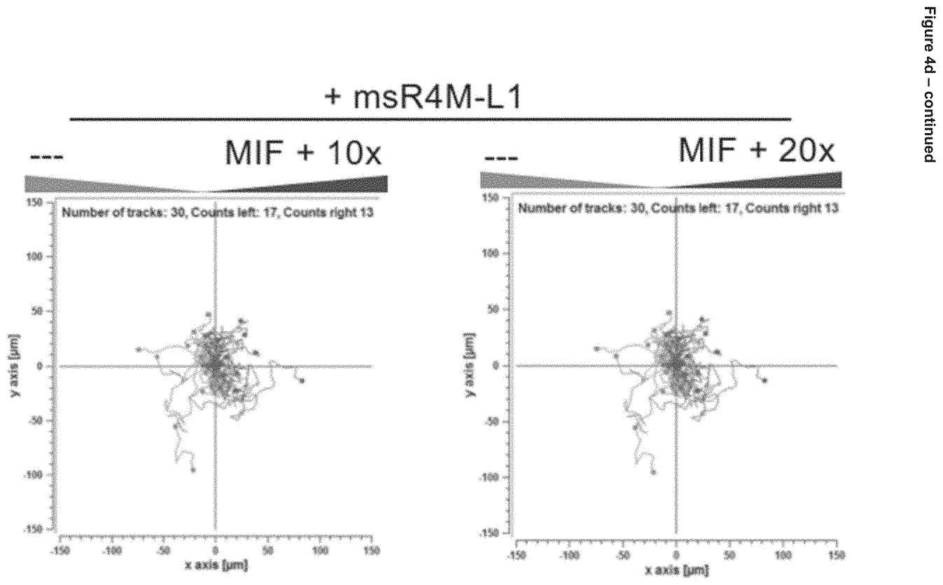

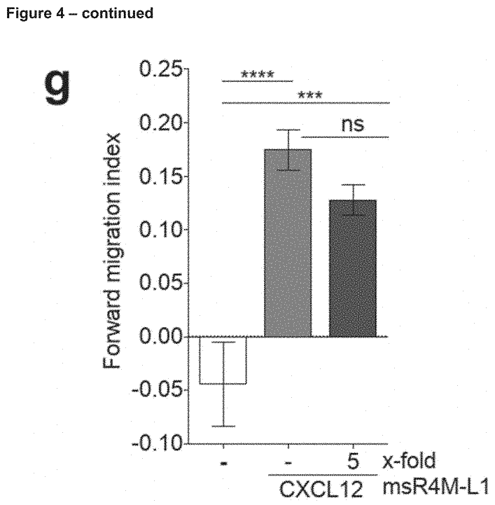

FIG. 4 CXCR4 ectodomain mimic msR4M-L1 specifically inhibits MIF- but not CXCL12-elicited pro-atherogenic monocyte activities. a-b MIF-specific Dil-LDL uptake in primary human monocyte-derived macrophages is dose-dependently inhibited by msR4M-L1 (indicated as molar excess over MIF). MIF was applied at a concentration of 80 nM. a Representative images of Dil-LDL-positive cells; b quantification (four-times-two independent experiments; 9 fields-of-view each). AMD3100 (AMD) was used to verify CXCR4 specificity of the MIF effect. c Same as in α-b, except that the small molecule inhibitor ISO-1 and neutralizing MIF antibody NIH/IIID.9 were used instead of msR4M-L1 (three-times-two independent experiments; 9 fields-of-view each: isotype control antibody IgG1: two-times-two). d Representative experiment demonstrating that msR4M-L1 inhibits MIF-elicited (red tracks) 3D chemotaxis of human monocytes as assessed by live-microscopic imaging of single cell migration tracks in x/y direction in μm. Increasing concentrations of msR4M-L1 (blue tracks, molar excess over MIF) as indicated; unstimulated control (grey tracks) indicates random motility. e Quantification of three independent experiments plotting the forward migration index. f A 5-fold molar excess of msR4M-L1 does not affect 3D human monocyte migration elicited by CXCL12; g quantification of f. Data in b, c, e, and g are reported as means±SD. Statistical analysis was performed with unpaired T-test and/or One-way ANOVA as appropriate (*P<0.05, **P<0.01, ***P<0.005, ****P<0.001).

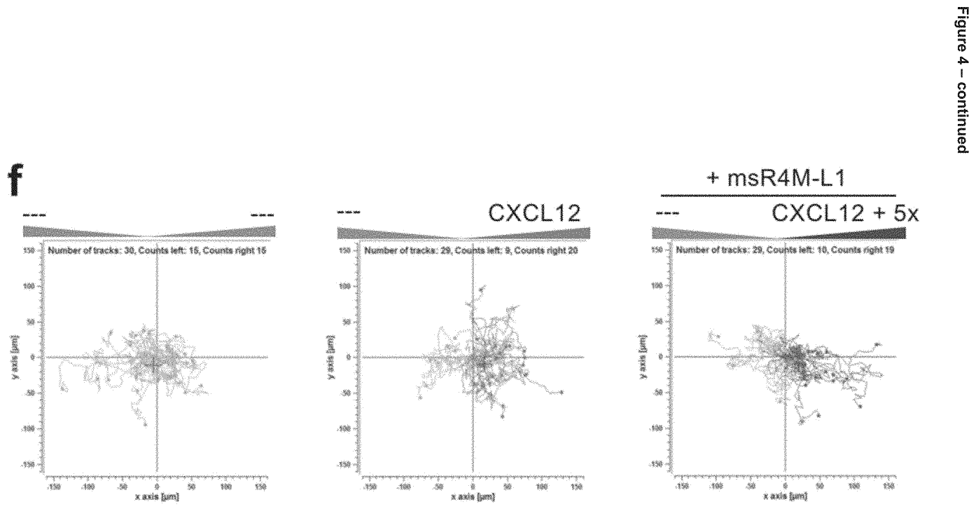

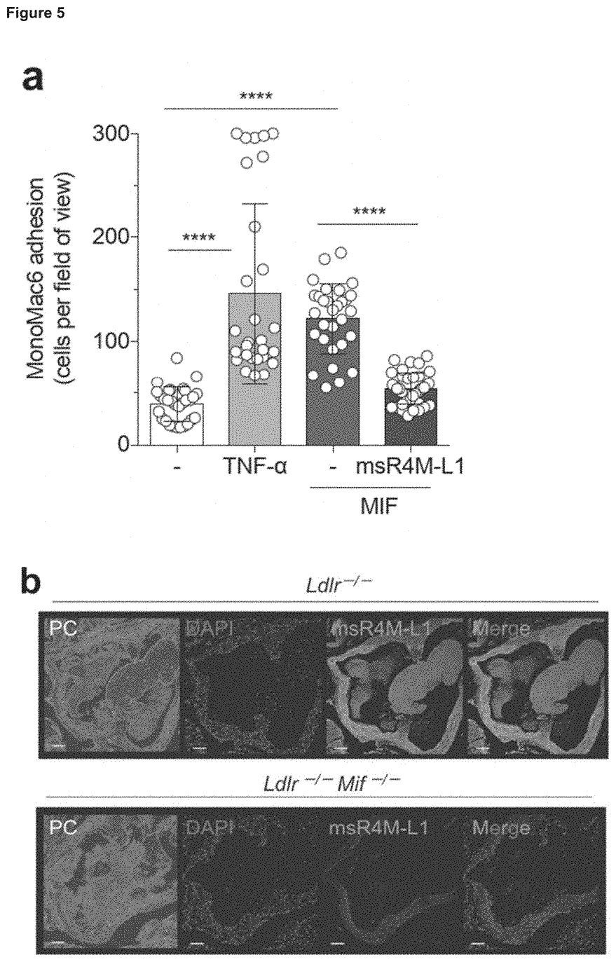

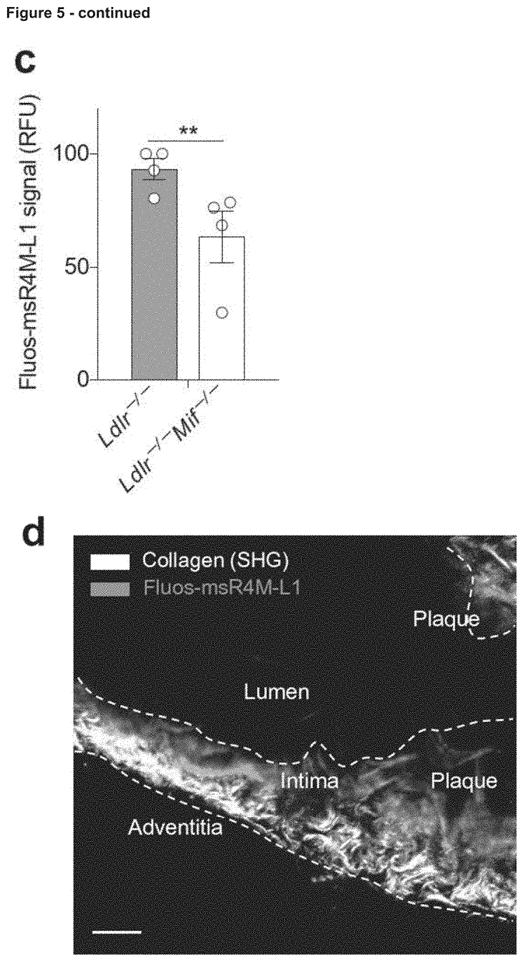

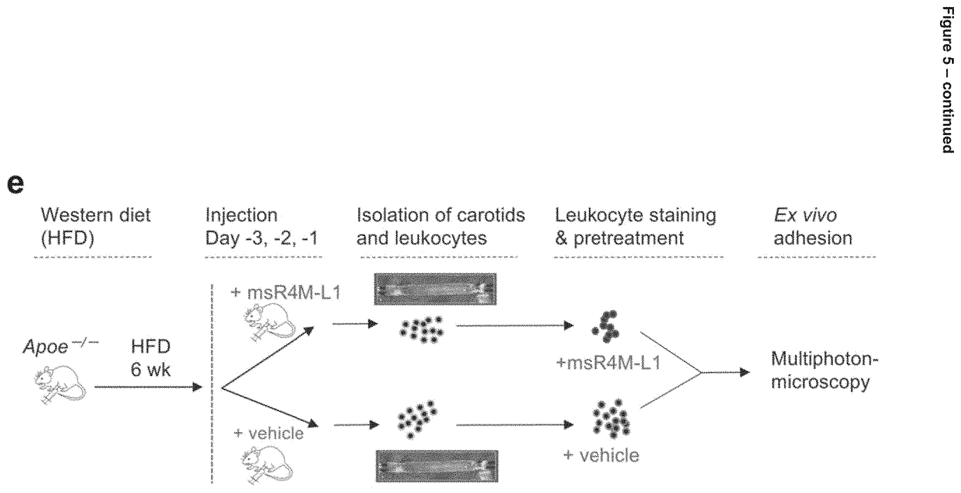

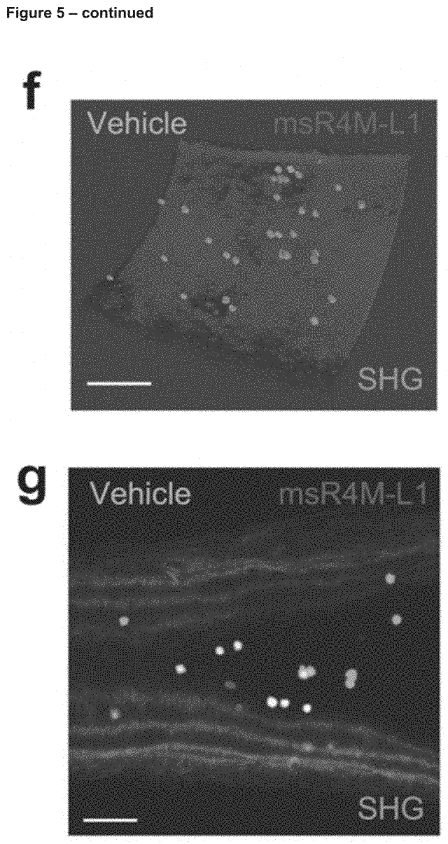

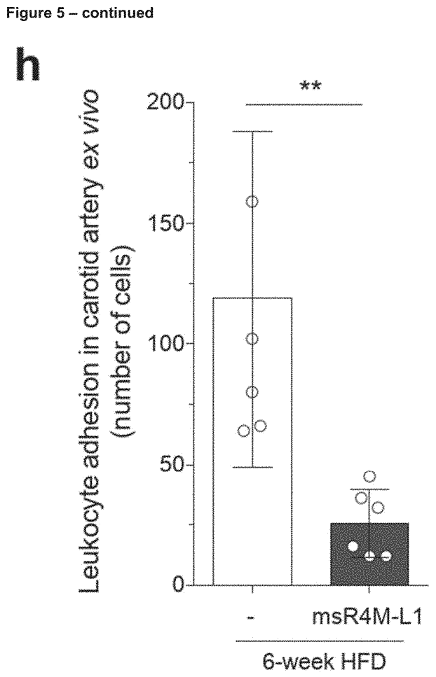

FIG. 5 CXCR4 ectodomain mimic msR4M-L1 localizes to atherosclerotic plaque tissue in a MIF-specific manner and inhibits atherogenic leukocyte arrest in vitro and in carotid arteries ex vivo. a MIF-induced static adhesion of MonoMac-6 monocytes to human aortic endothelial cells (HAoECs) is ablated by msR4M-L1. TNF-α served as a positive control (3×10 independent fields-of-view each). b-c Fluos-msR4M-L1 stains aortic root sections from atherogenic Ldlr^{-/-} mice on HFD in a MIF-specific manner (comparison between specimens from Ldlr^{-/-} and Ldlr^{-/-} Mif^{-/-} mice). b Representative images (PC, phase contrast; DAPI, cell nuclei); c quantification (as relative fluorescence units) from n=4 experiments indicates MIF-specific staining over background. d Multiphoton laser-scanning microscopy (MPM) image of a whole-mount carotid artery prepared from an Apoe^{-/-} mouse on HFD, showing that in-vivo-administered Fluos-msR4M-L1 localizes to atherosclerotic plaques. Second harmonic generation (SHG) applied for visualization of vessels. e-h msR4M-L1 inhibits leukocyte adhesion in atherogenic carotid arteries under flow as analyzed by MPM. e Schematic summarizing the ex-vivo leukocyte adhesion experiment. msR4M-L1 or vehicle was injected before vessel harvest as indicated; flushed leukocytes were pre-treated accordingly and are stained in red (msR4M-L1; CMPTX) or green (vehicle; CMFDA). f Representative image of a carotid artery (vessel morphology revealed by SHG: collagen, bright blue, Tunica adventitia; elastin, blue, Tunica media), showing that pre-treatment with msR4M-L1 (red) leads to reduced luminal leukocyte adhesion compared to vehicle control (green), imaged by 3D reconstruction after Z-stacking (0.8-1.5 μm). g Still image of a z sectioning video scan (single field of view) through the artery. h Quantification based on measurements with 5-6 independent carotid arteries per group. The number of luminally adhering cells is plotted. Scale bars: b, 100 μm; d, 50 μm; f. 100 μm; g, 100 μm. Data in a, c and h are reported as means±SD. Statistical analysis was performed with unpaired T-test (**P<0.01, ****P<0.001).

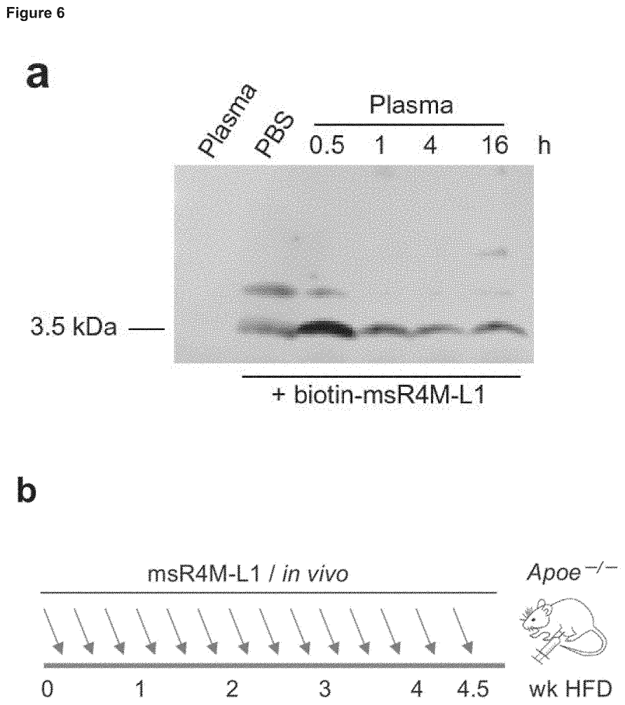

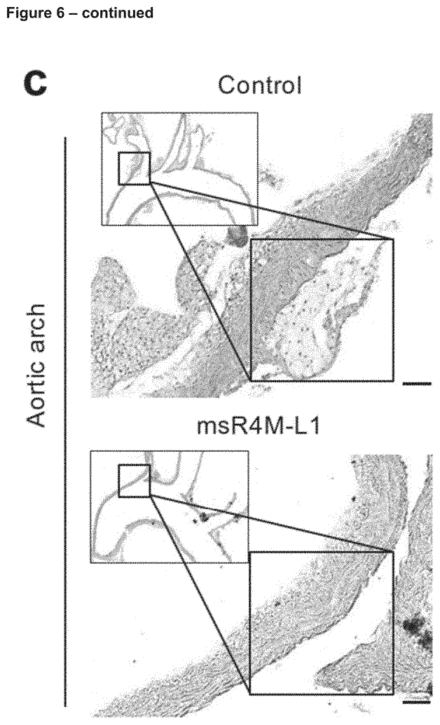

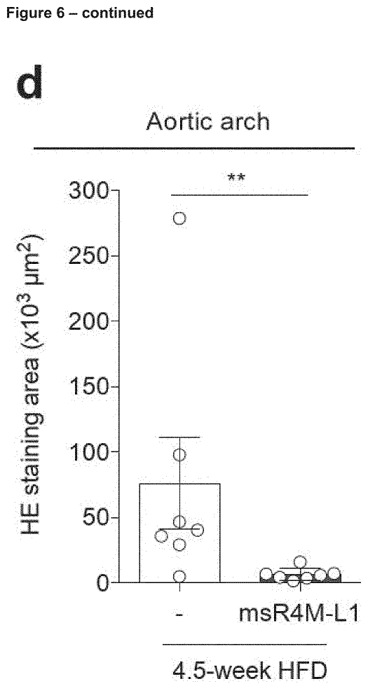

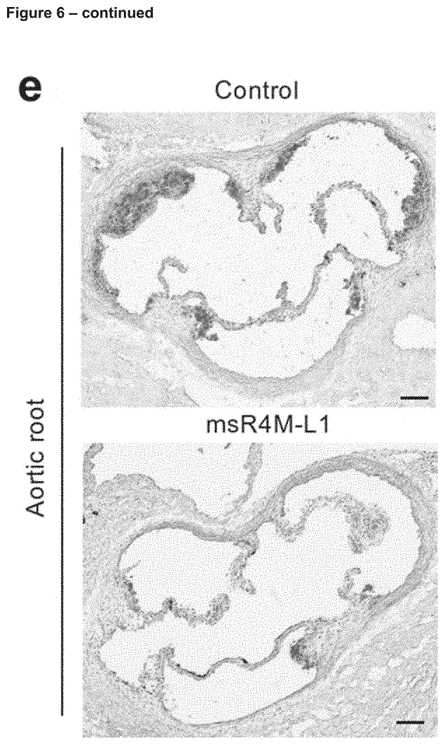

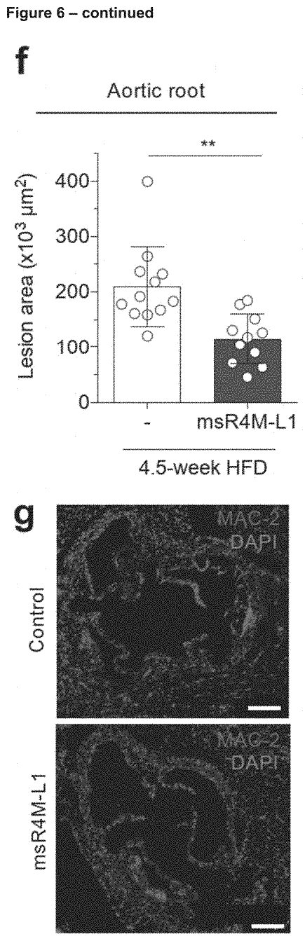

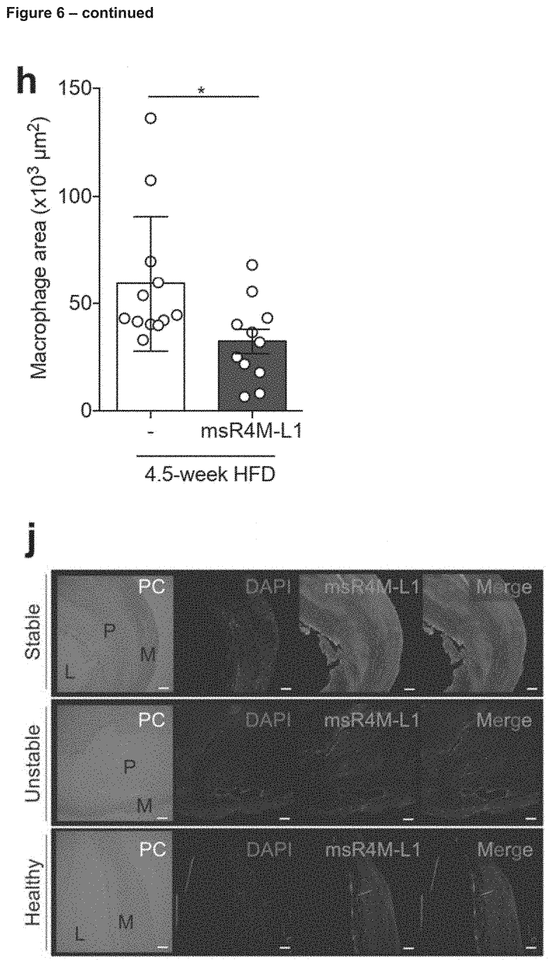

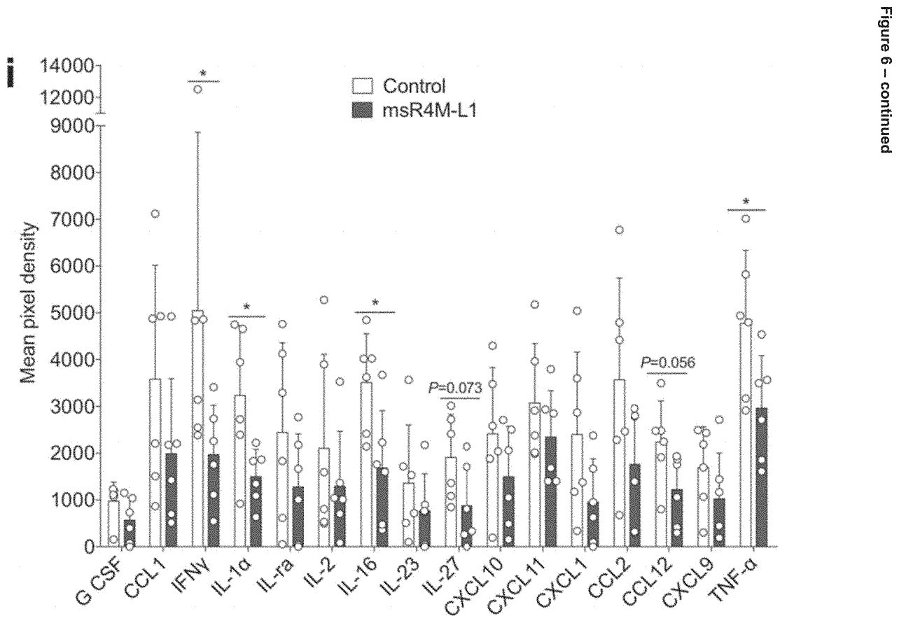

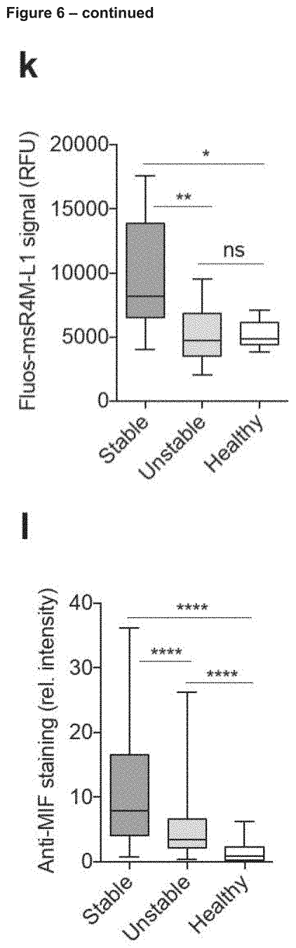

FIG. 6 The CXCR4 mimic msR4M-L1 inhibits atherosclerosis and inflammation in vivo and msR4M-L1-based staining mirrors the MIF staining phenotype of plaques from human carotid arteries. a Streptavidin-POD-developed Western blot of biotin-msR4M-L1 incubations exposed to human plasma verifies proteolytic stability of the engineered peptide of up to 16 h (PBS, 16 h control). b Schematic showing the in-vivo-injection regimen for the peptide in atherogenic Apoe^{-/-} mice on HFD. c-d msR4M-L1 treatment reduces atherosclerotic lesions in aortic arch. Representative images (c) and quantification (d, 7 mice per group) of HE-stained sections from msR4M-L1- versus vehicle-treated mice. e-f msR4M-L1 treatment reduces atherosclerotic lesions in aortic root. Representative images (e) and quantification (f, 12 mice per group) of oil red O-stained sections from msR4M-L1- versus vehicle-treated mice. g-h msR4M-L1 treatment reduces lesional macrophage content in aortic root. Representative images (g) and quantification of macrophage area (h, 11-12 mice per group) of anti-MAC2-stained (red) sections from msR4M-L1- versus vehicle-treated mice (DAPI, blue). i msR4M-L1 reduces circulating inflammatory cytokine levels. Analysis by mouse cytokine array featuring 40 inflammatory/atherogenic, cytokines/chemokines on plasma samples from msR4M-L1- versus vehicle-treated Apoe^{-/-} mice on HFD. Data are means±SD from six mice per group, performed in duplicate each. j-k Fluos-msR4M-L1 preferentially stains stable human carotid atherosclerotic plaque sections obtained from patients undergoing carotid endarterectomy (CEA). Representative images (i; PC, phase contrast; DAPI for cell nuclei) and quantification or the Fluos-msR4M-L1 signal (as relative fluorescence units) from n=9 stable and n=15 unstable plaque specimens and n=6 healthy vessels. l Anti-MIF antibody preferentially stains stable CEA plaques (n=11 stable and n=15 unstable plaque specimens, n=6 healthy vessels; n=5-7 random sections from each specimen). Scale bars: c, 50 μm; e, 200 μm; g, 200 μm; j, 200 μm. Data in d, f and h are reported as means±SD, data in j and k as box-whisker plot. Statistical analysis was performed with unpaired T-test (d, f, h, i). Mann-Whitney U-test or Kruskal-Willis test using ANOVA (i, k, l) as appropriate (*P<0.05, **P<0.01, ***P<0.005).

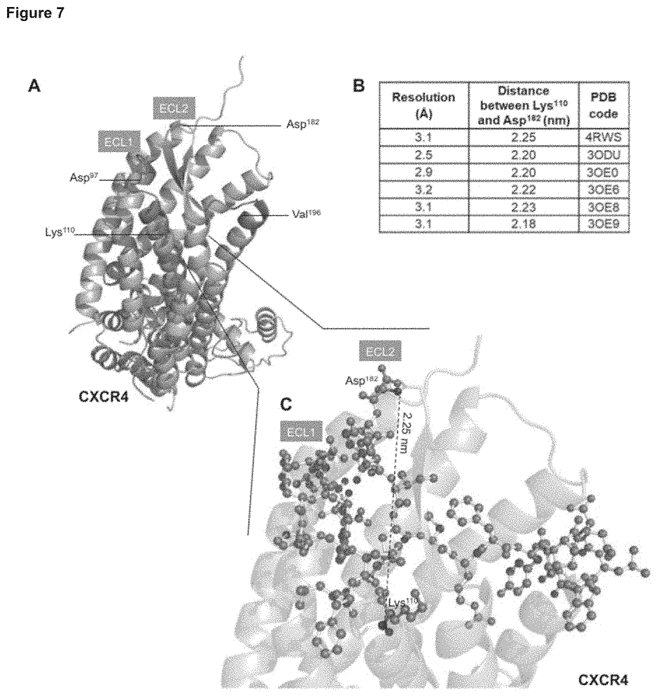

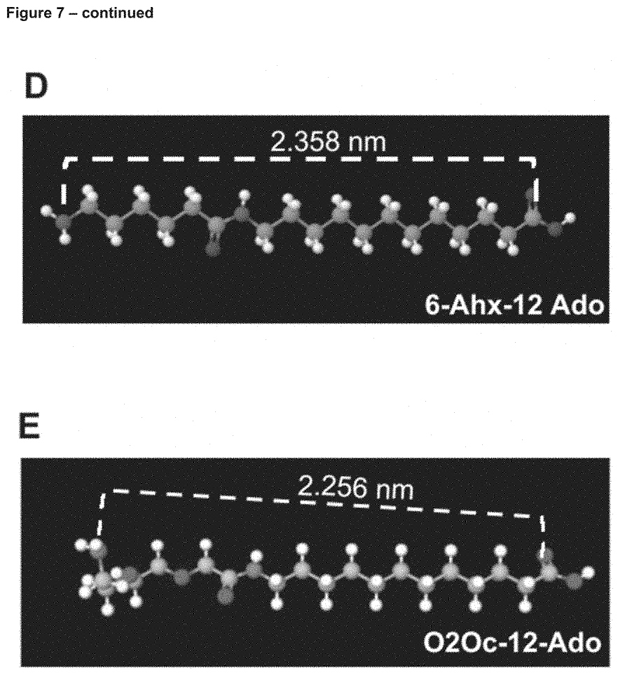

FIG. 7 Structural basis for the design of the linker connecting ECL1 and ECL2 peptide sequences of human CXCR4. a Ribbon structure of human CXCR4 based on the crystal structure according to PDB code 4RWS¹. Sequence stretches of extracellular loops ECL1 and ECL2 that were found to interact with MIF according to peptide array mapping² are highlighted in blue, and the N- and C-terminal residues of the ECL1 and 2 stretches are indicated. b Overview of distances between Lys¹⁰ and Asp¹⁸² of CXCR4 according to different crystal structure models^{1, 3}. The respective resolutions of the structures and the PDB codes are indicated. c Zoomed top view of the CXCR4 ectodomain according to a; the measured length between the C-terminus of Lys¹¹⁰ and the N-terminus of Asp¹⁸² (2.25 nm) is shown. d, e Lengths of the 6-Ahx-12-Ado and O2Oc-12-Ado linkers as used in msR4M-L1 (d) and msR4M-L2 (e), respectively.

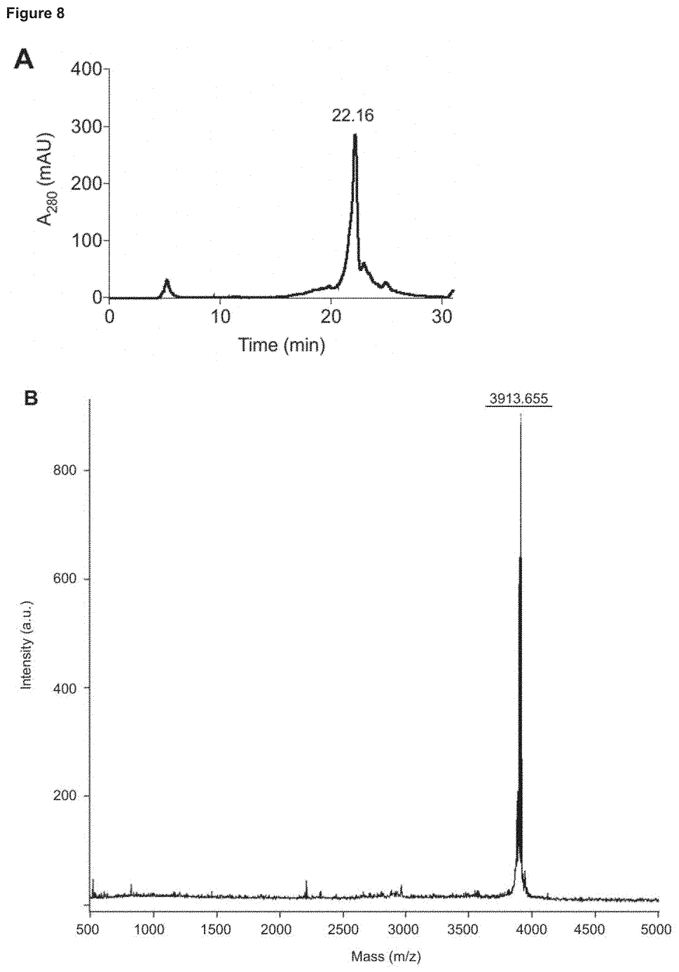

FIG. 8 Purification of msR4M-L1 by HPLC and verification of peptide purity by mass spectrometric analysis. a Representative C18 HPLC chromatogram (absorbance 280 nm) of msR4M-L1 (retention time: 22.16 min; crude product) following solid phase peptide synthesis (SPPS). b Matrix-assisted laser desorption/ionization mass spectrometry (MALDI-MS) spectrum of HPLC-purified msR4M-L1. The theoretical calculated mass [M+H]⁺ is 3912.92: the experimental mass [M+H]⁺ is 3913.66.

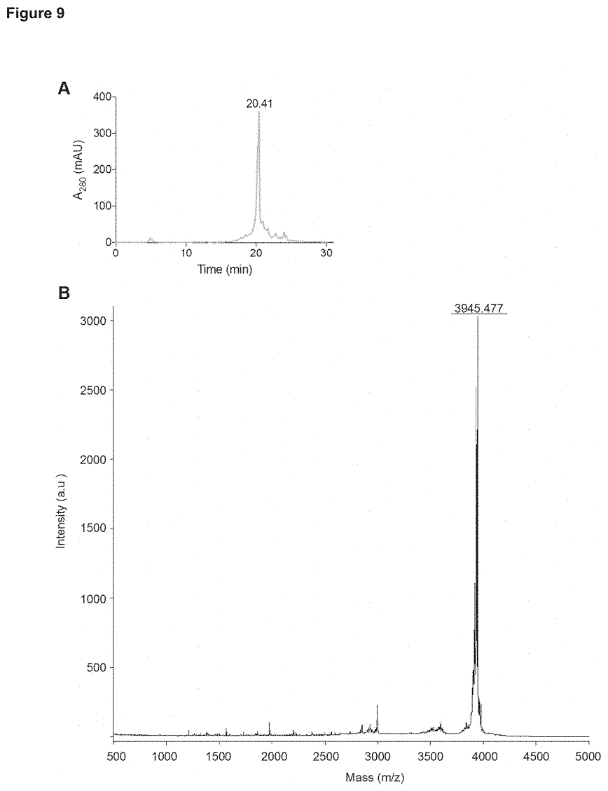

FIG. 9 Purification of msR4M-L2 by HPLC and verification of peptide purity by mass spectrometric analysis. a Representative C18 HPLC chromatogram (absorbance 280 nm) of msR4M-L2 (retention time: 20.41 min; crude product) following solid phase peptide synthesis (SPPS). b Matrix-assisted laser desorption/ionization mass spectrometry (MALDI-MS) spectrum of HPLC-purified msR4M-L2. The theoretical calculated mass [M+H]⁺ is 3944.93: the experimental mass [M+H]⁺ is 3945.48.

FIG. 10 Binding affinities between CXCR4 ectodomain peptide mimics and MIF versus CXCL12 as determined by fluorescence spectroscopic titration. Fluorescently labeled (FLUOS) ectodomain peptides (linked peptides or single extracellular loop (ECL) peptides) were titrated with increasing concentrations of MIF or CXCL12; conversely for MIF, fluorescently labeled (Alexa-488) MIF was titrated with increasing concentrations of ectodomain peptides. Left panels of a-q Fluorescence emission spectra. Right panels of a-q Binding curves derived from the fluorescence emission at 519 nm (Alexa-488 MIF) or 522 nm (Fluos-labeled peptides) as indicated. Binding affinities (K_{D}) were derived from these curves (see list of K_{D} values in Table 1 of the main manuscript). a-l Ectodomain peptide/MIF titrations as indicated; m-q ectodomain peptide/CXCL12 titrations; a, c, e, g, i, k Fluos-labeled ectodomain peptide/MIF titrations; b, d, f, h, j, l ectodomain peptide/Alexa-MIF titrations; m-q Fluos-labeled ectodomain peptide/CXCL12 titrations. Panels a and m are identical with FIGS. 1c and d of the main manuscript, respectively, and are included in this Figure for clarity reasons. Data shown are means±SD from three independent titration experiments.

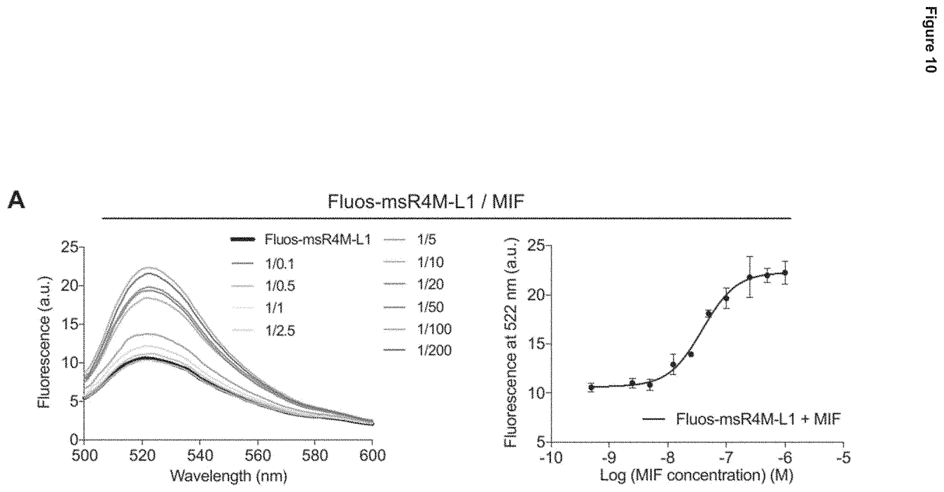

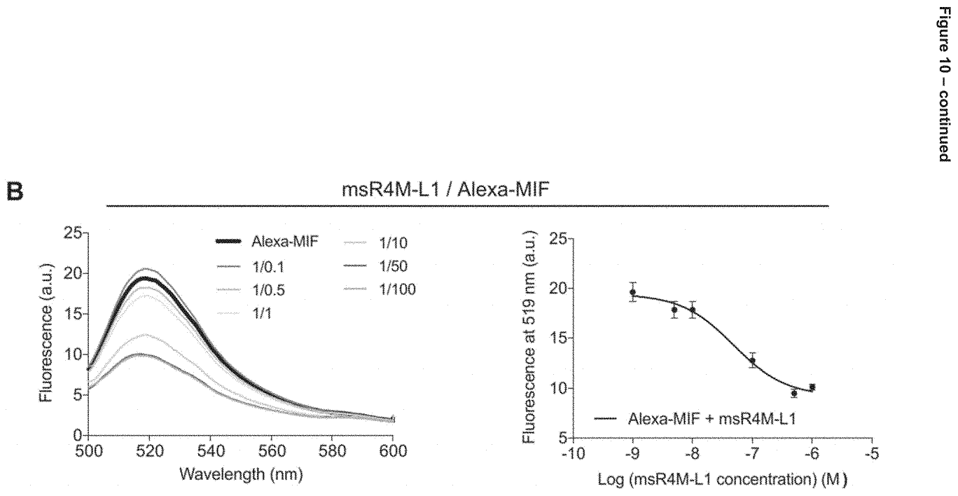

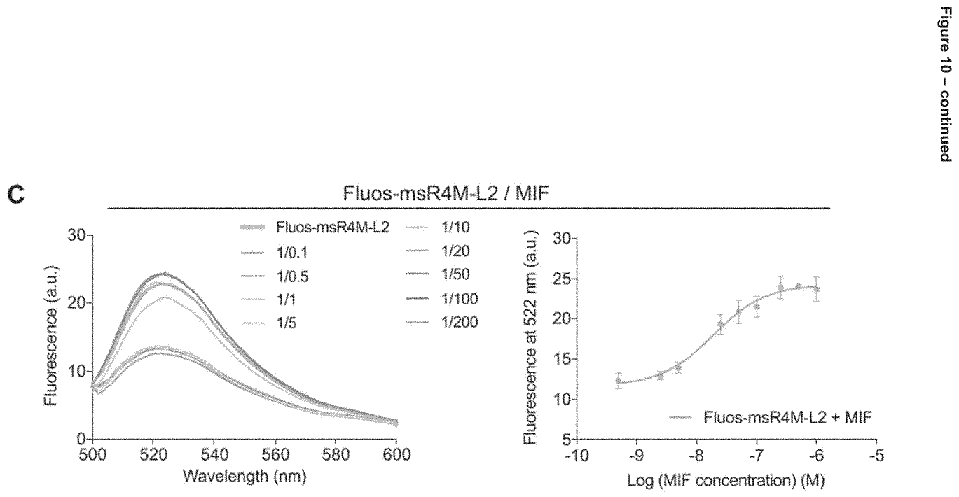

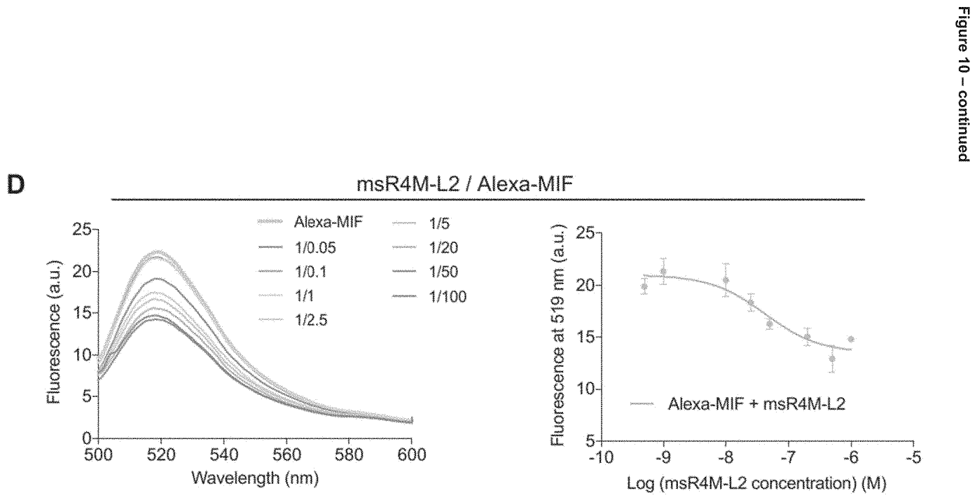

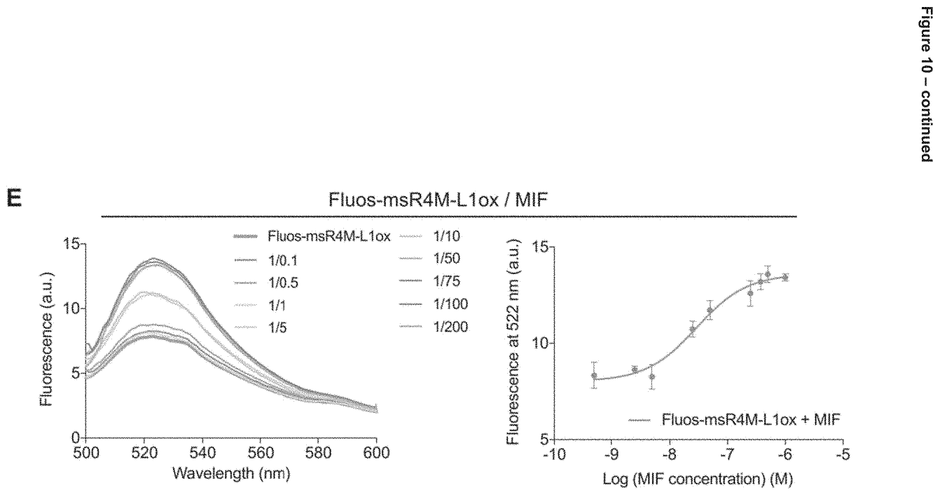

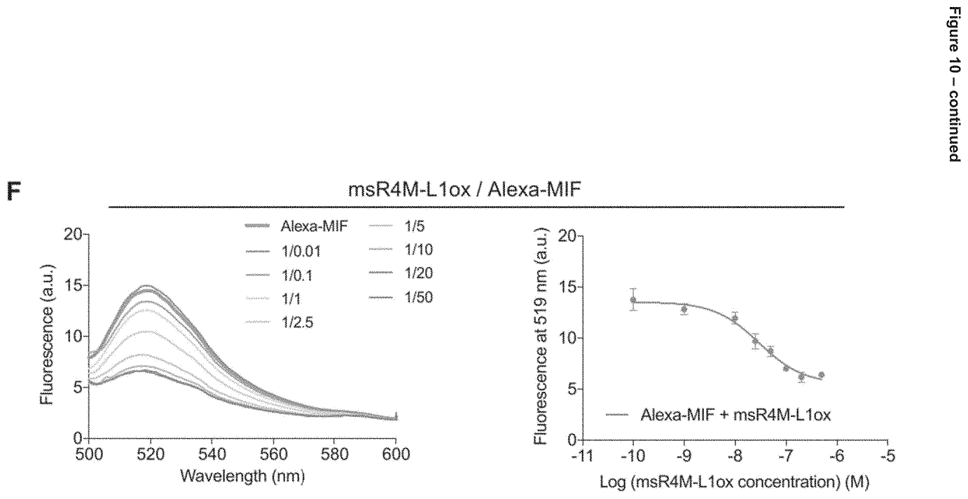

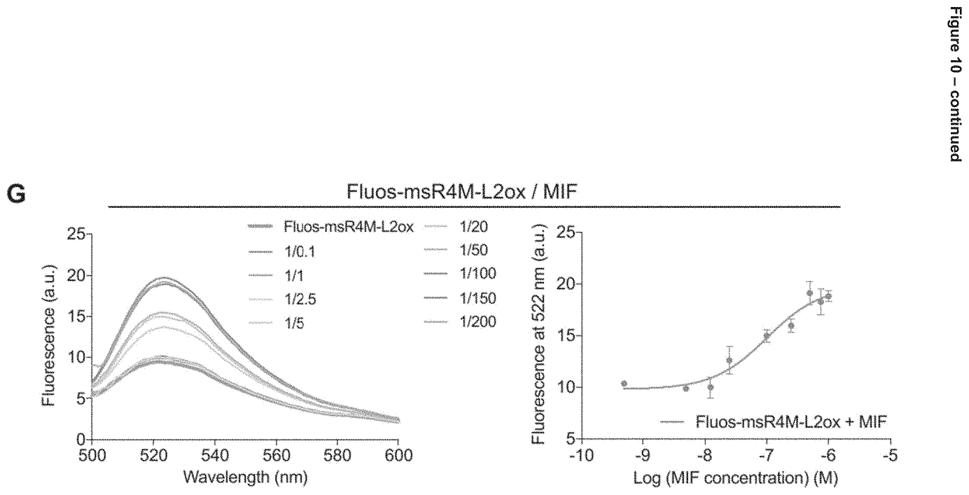

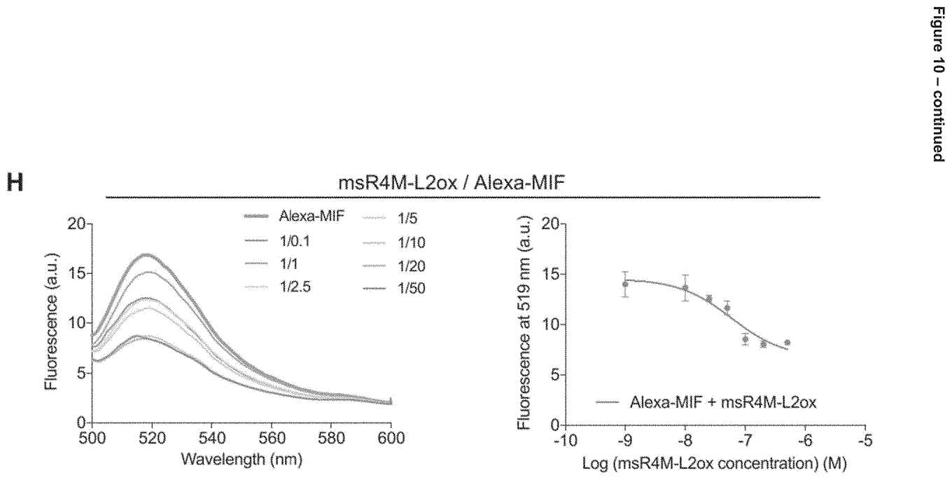

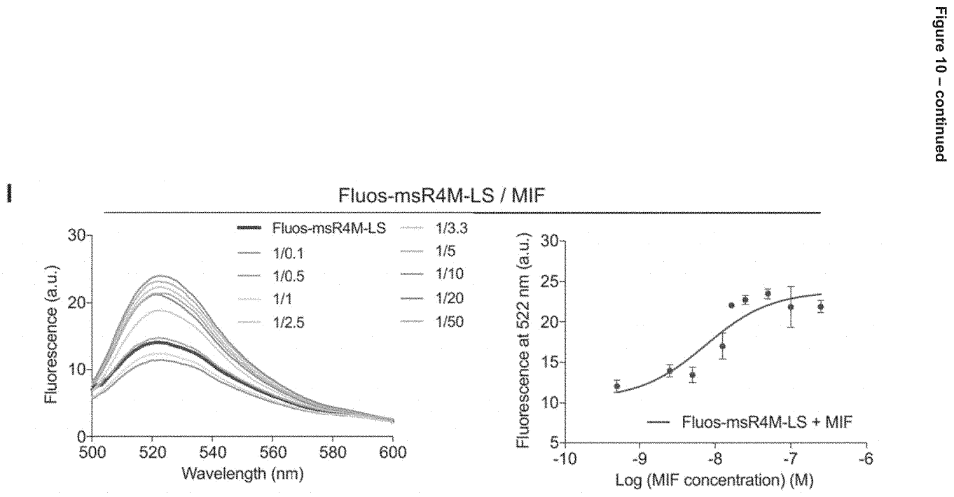

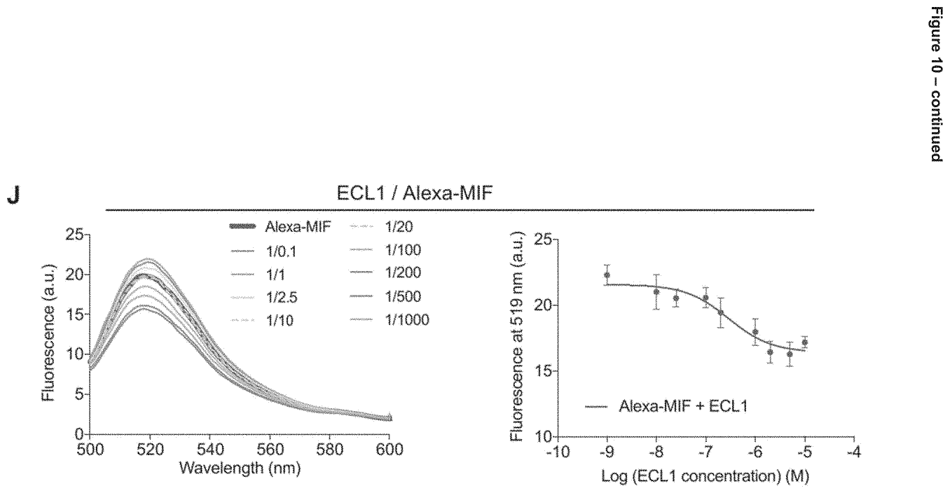

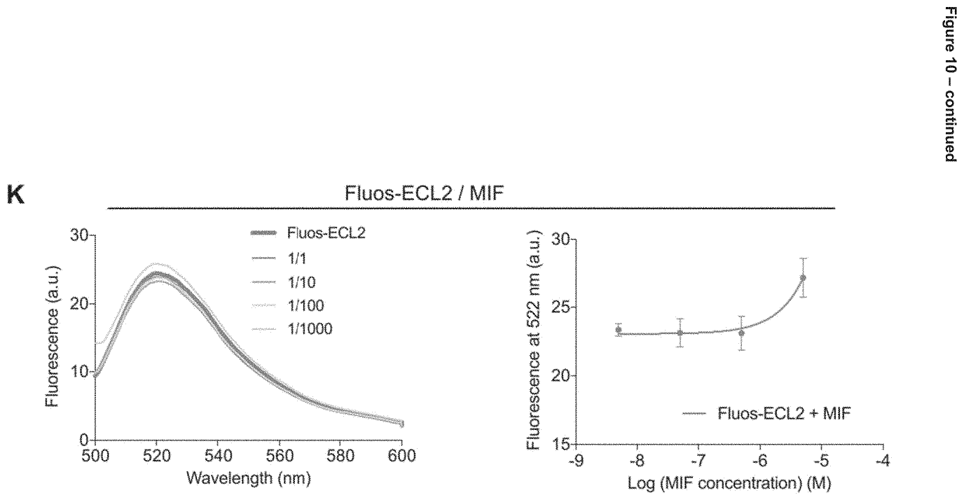

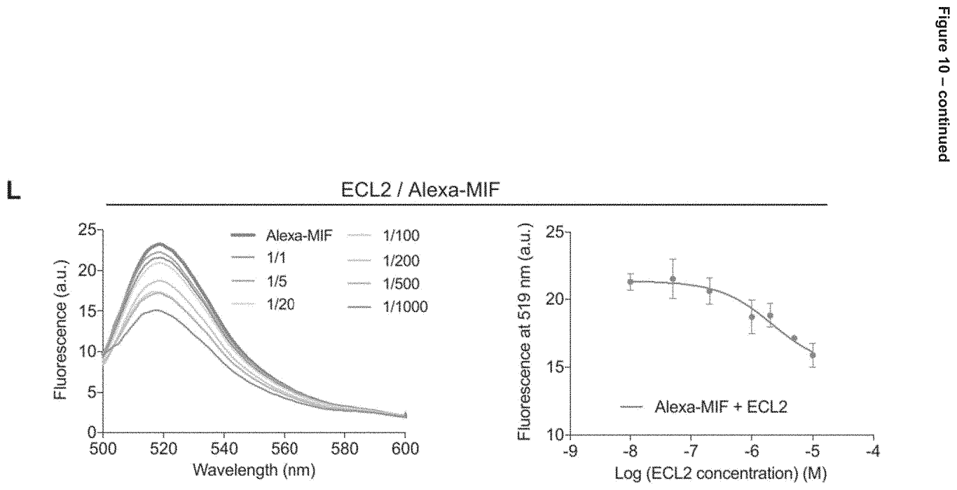

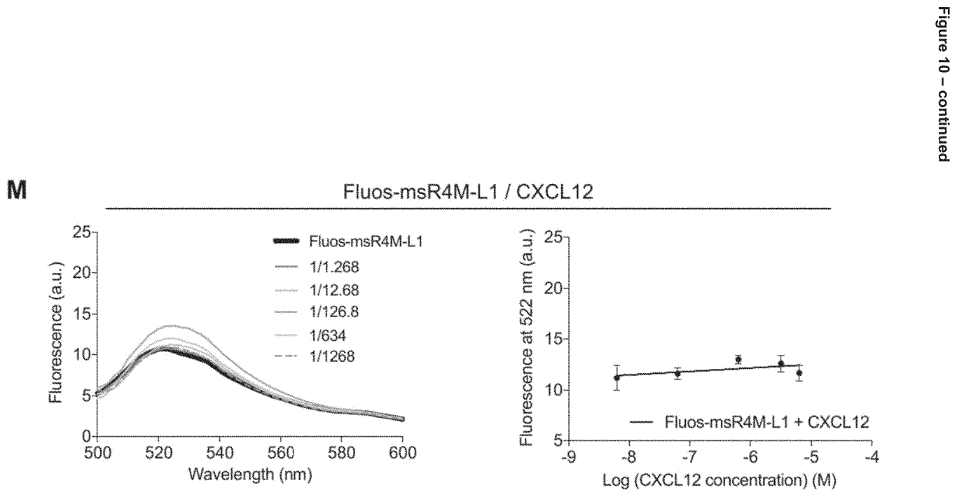

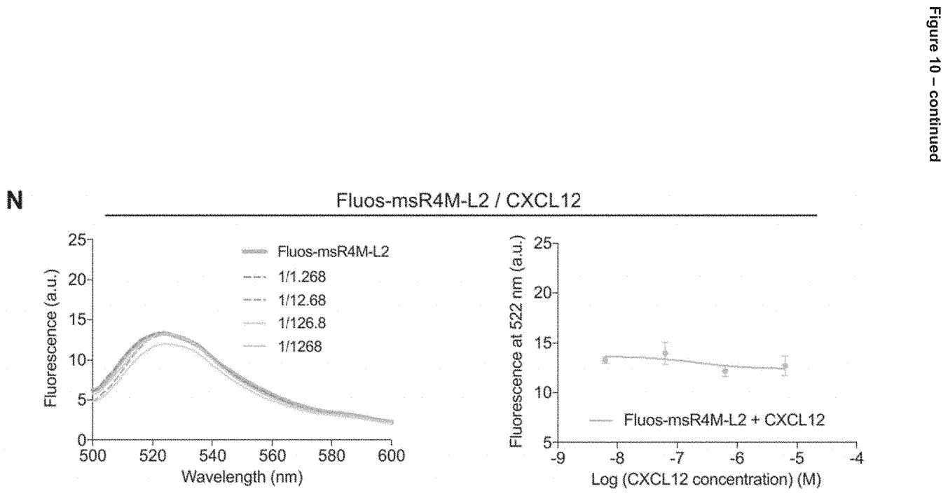

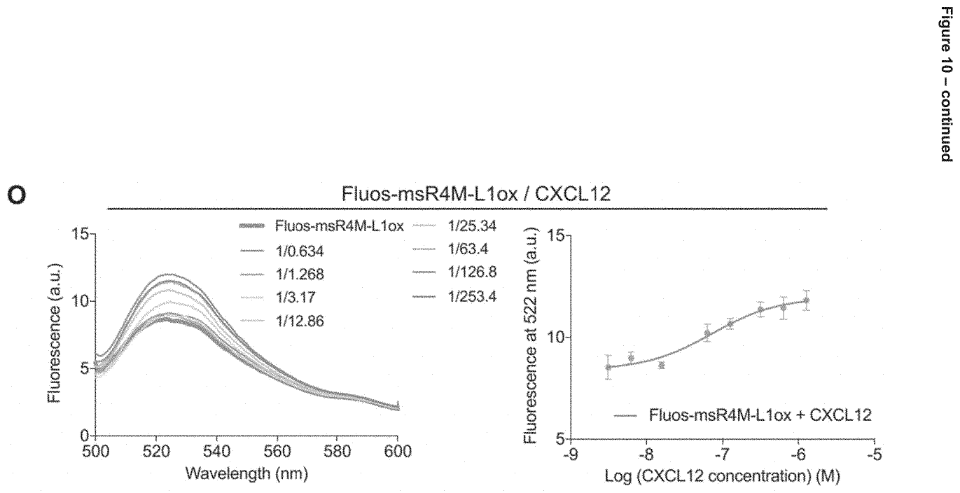

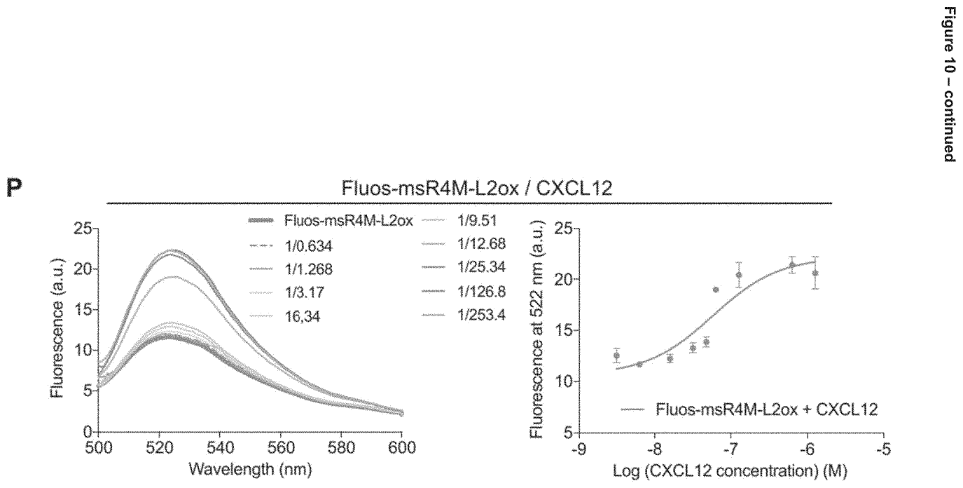

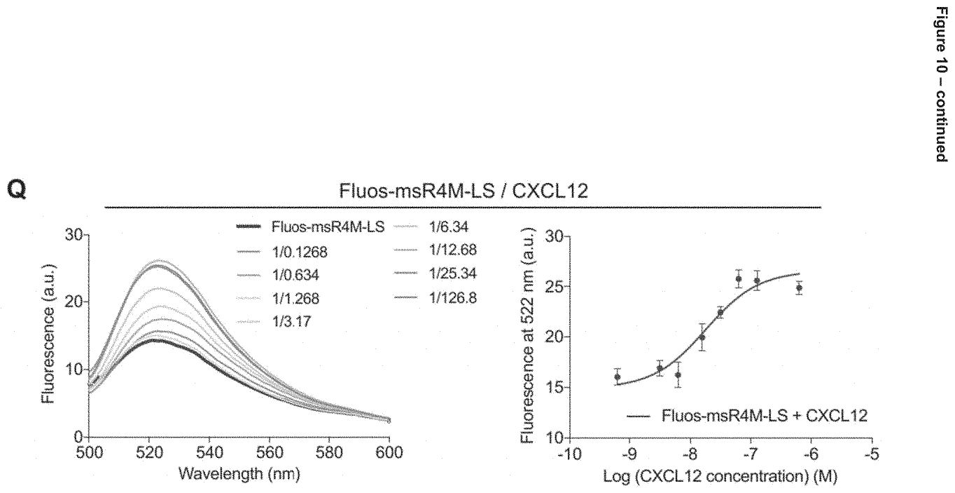

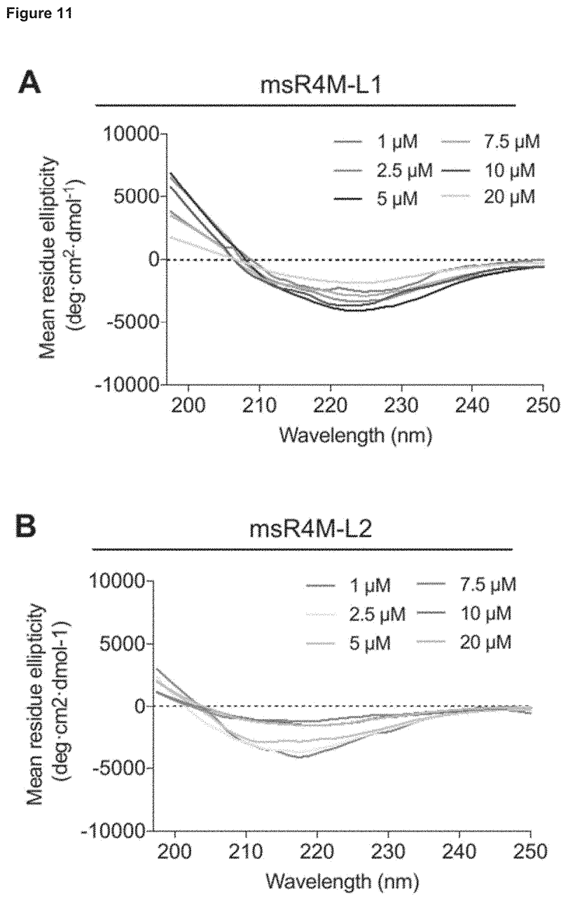

FIG. 11 Self-assembly propensities of msR4M-L1 versus msR4M-L2 as determined by circular dichroism (CD) spectroscopy and fluorescence spectroscopic titration. a, b CD spectra of msR4M-L1 (a) and msR4M-L2 (b) at increasing concentrations (1, 2.5, 5, 7.5, 10 and 20 μM). The more pronounced concentration-dependent reduction in CD signal for msR4M-L2 (b) compared to msR4M-L1 (a) in the region between 210 and 225 nm indicates a higher aggregation propensity of msR4M-L2. Conformations in the CD spectra were measured as mean residue ellipticity (MRE) as a function of the wavelength (given in nm) in the far-UV range. c, d Determination of self-assembly propensity by fluorescence titration spectroscopy. Binding (‘self-assembly’) was analyzed by titrating fluorescently labeled (Fluos) msR4M-L1 and msR4M-L2 with increasing concentrations of unlabeled msR4M-L1 (c) and msR4M-L2 (d), respectively. Left panels of c,d Fluorescence emission spectra. Right panels of c,d Binding curves derived from the fluorescence emission at 522 nm. Data are means±SD of three independent titration experiments.

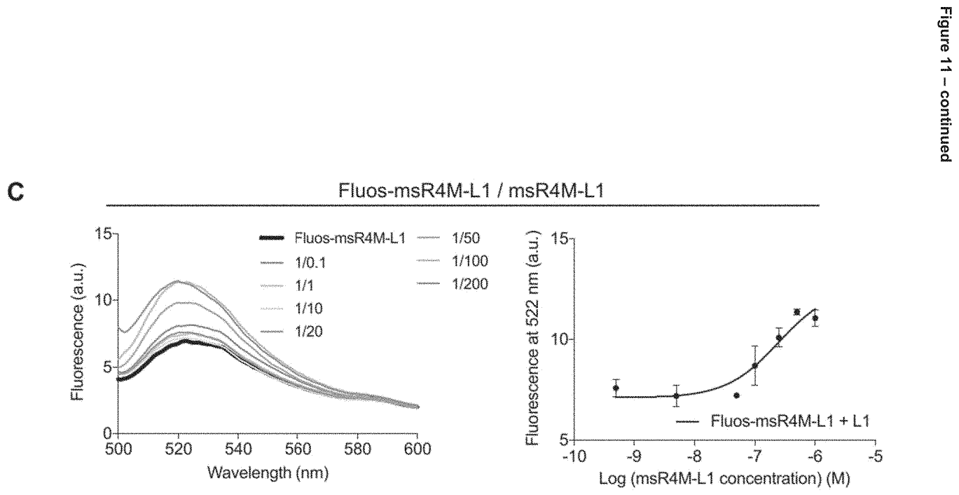

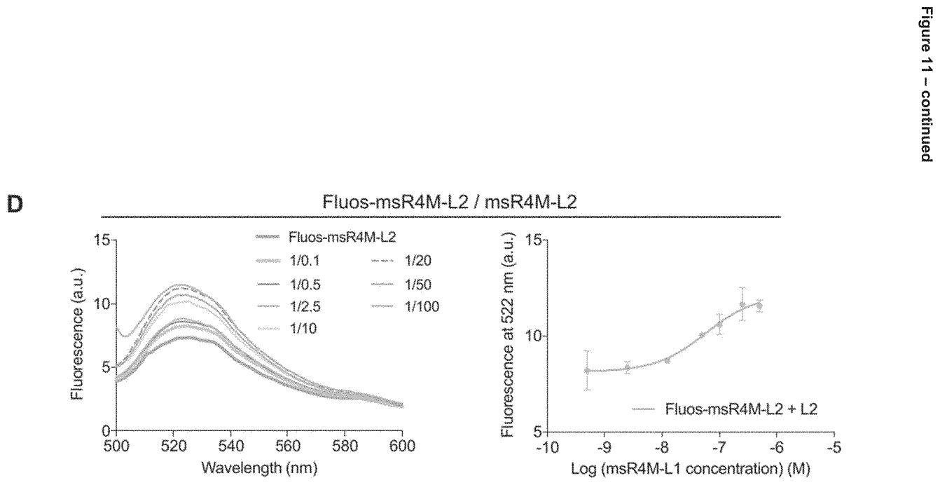

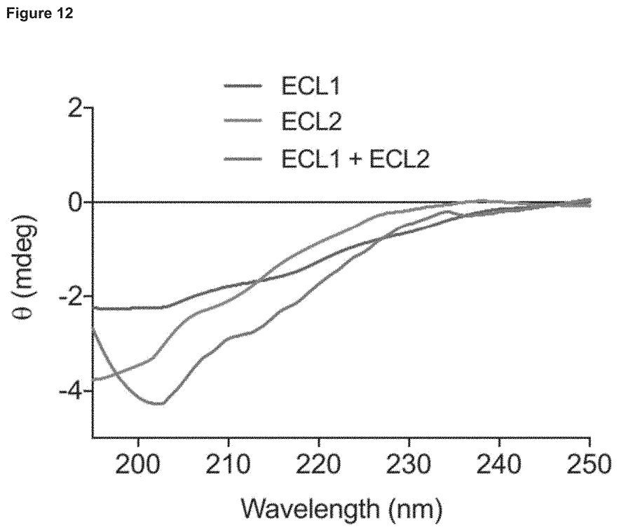

FIG. 12 Circular dichroism (CD) spectroscopy reveals that non-linked mixtures of CXCR4 ectodomain loop sequences ECL1 and ECL2 exhibit mostly random coil conformation in contrast to the linked mimics msR4M-L1 and msR4M-L2. CXCR4 ectodomain peptides ECL1[97-110] and ECL2[182-196] were mixed at a 1/1 ratio and subjected to far-UV CD spectroscopy. Corresponding individual ECL1 and 2 peptides were measured for comparison. CD spectra are represented as degrees of ellipticity (θ mdeg) as a function of the wavelength (given in nm) in the far-UV range.

FIG. 13 Overview of the MIF sequences involved in binding to the CXCR4 ectodomain peptide msR4M-L1. MIF sequences with high binding affinity are colored in blue, non-binders/low affinity-binders are depicted in grey. The scheme visualizes the binding affinities (K_{D}) between msR4M-L1 and partial MIF peptides as determined by fluorescence titration spectroscopy as listed in detail in Table 2. a Coverage of the full-length MIF sequence highlighting the core binding region 38-80 (blue) and the tested, non-binding, peptides located N- and C-terminal of this region (grey). b Focus on region 38-80. Binding (blue) versus non-binding/low-affinity binding (grey) partial MIF sequences within this region are aligned. For the purpose of this overview scheme, binding is defined relative to the affinity between msR4M-L1 and full-length MIF (KD≈30 nM): binding/high-affinity binding as “within a range of 10× K_{D} of msR4M-L1” and non-binding/low-affinity binding as “more than 100× less affine than the K_{D} of msR4M”.

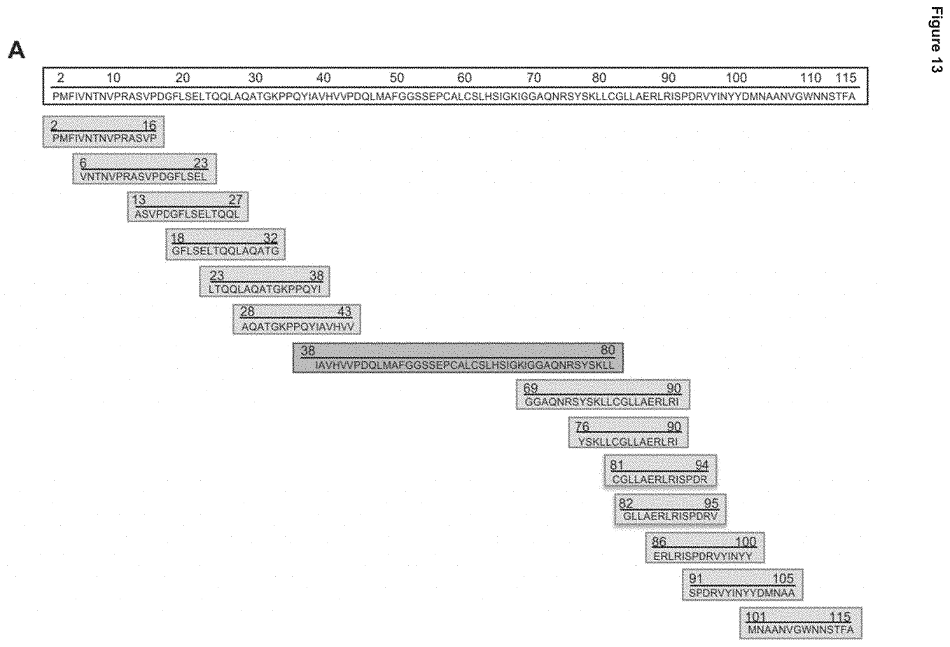

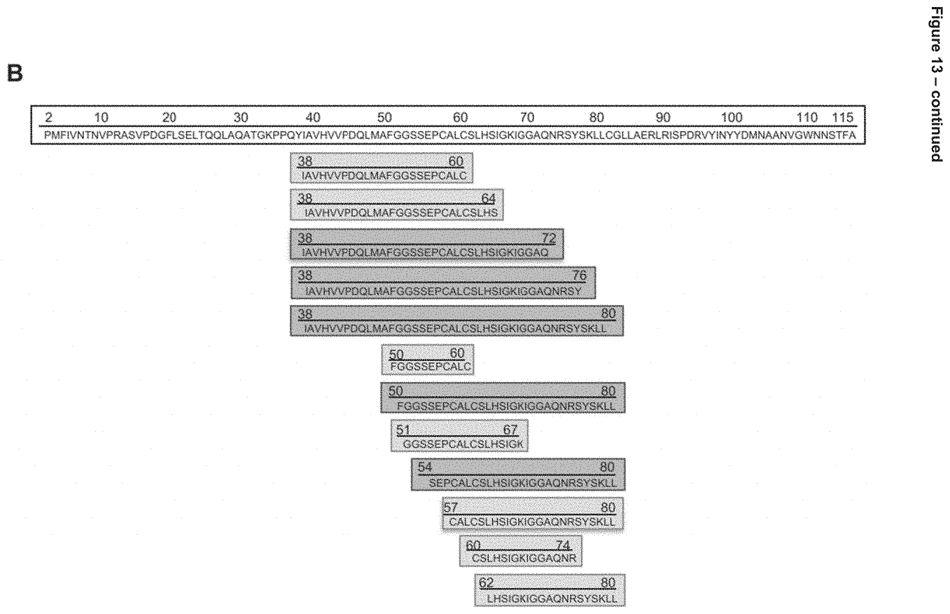

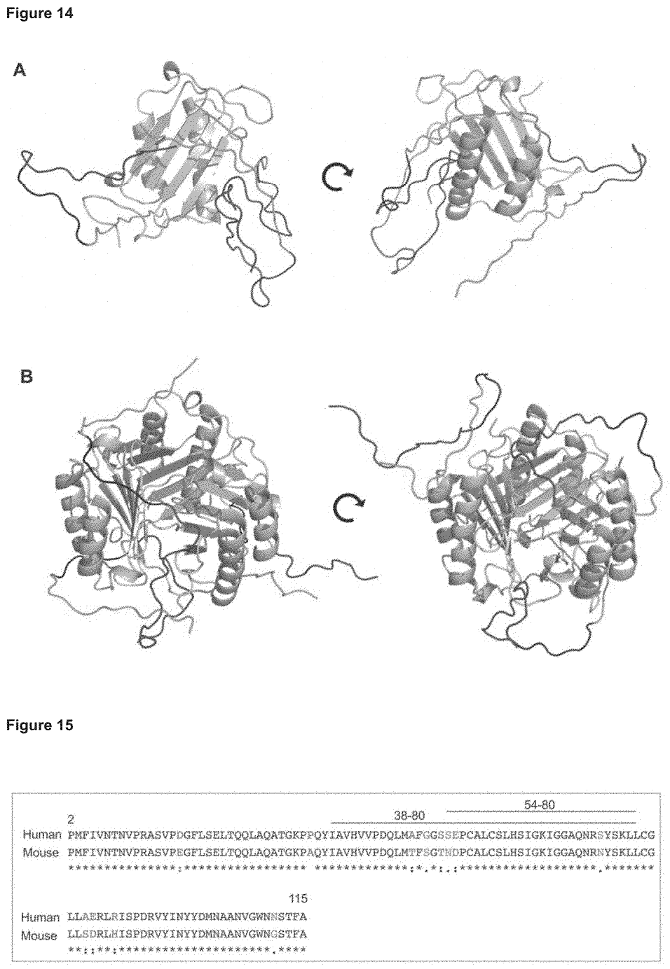

FIG. 14 Molecular docking between the msR4M-L1 homolog CXCR4-ECL1-[97-110]-Gly_{(7)-ECL}2[182-196] and MIF. Molecular docking confirms possible interaction sites between MIF and msR4M-L1. Interactions were investigated by protein-peptide-docking approaches using the structure of full-length human MIF (grey) and CXCR4-ECL1-[97-110]-Gly_{(7))-ECL}2[182-196], an msR4M-L1-like CXCR4 ectodomain mimic with a heptaglycine linker instead of the 6-Ahx-12-Ado spacer, which cannot be analyzed itself in the docking program but has essentially the same length as the 6-Ahx-12-Ado spacer. The ECL1 part of the ectodomain mimic is colored in green, the ECL2 part in blue and the glycine linker in black. The structure of MIF is from PDB 3DJH and docking was performed using the HPEPDOCK server (http-Jhuanglab.phys.hust.edu.cn/hpepdock). a Three high-ranking docking models for the complex between ‘monomeric’ MIF and CXCR4-ECL1-[97-110]-Gly₍₇₎-ECL2[182-196]. b Three high-ranking docking models for the complex between ‘trimeric’ MIF and CXCR4-ECL1-[97-110]-Gly₍₇₎-ECL2[182-196]. Selected models in which the msR4M-L1-like ectodomain mimic is arranged in close vicinity (<0.4 nm) to residues belonging to the N-like loop region of MIF are shown.

FIG. 15 High degree of sequence identity between human and mouse MIF and equal binding affinity of msR4M-L1 to human and mouse MIF. The high degree of sequence identity between human and mouse MIF is also represented in the core msR4M-L1 binding region at sequence 38-80 or 54-80. Sequence alignment between the protein sequences of human and mouse MIF using the multiple sequence alignment tool ClustalW2 (EMBL-EBI, https://www.ebi.ac.ukITools/msa/clustalw2/). Conserved residues are marked with an asterisk (*), conservative replacements by a colon (:) and blue color, semi-conservative replacements by a half-colon (.) and green color, and non-conservative replacements by empty denotation ( ) and red color.

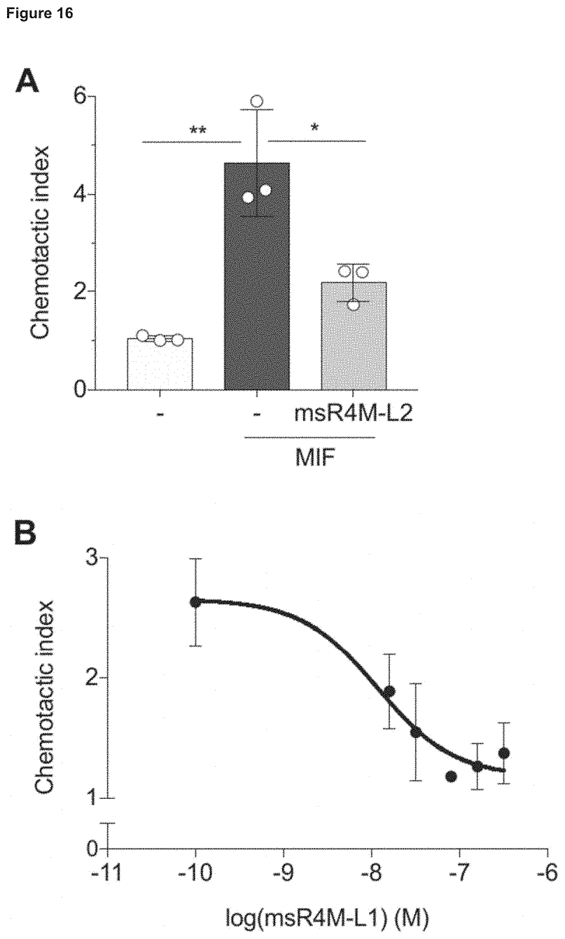

FIG. 16 Inhibition of MIF-triggered primary mouse B-lymphocyte chemotaxis by msR4M-L2 and msR4M-L1. MIF was added to the lower chamber of a Transwell device as chemoattractant at a concentration of 16 nM. a msR4M-L2 inhibits MIF-triggered B-lymphocyte chemotaxis. msR4M-L2 was added at a 5-fold molar excess over MIF. b Concentration-dependent inhibition of MIF-mediated B-lymphocyte chemotaxis by msR4M-L1. The chemotactic index of MIF-elicited chemotaxis is plotted against the different concentrations of msR4M-L1. IC50 plot with the chemotactic index plotted against the log(concentration) of msR4M-L1. The deduced estimated IC50 value is 10 nM. The data shown are means±SD of three experiments. Statistical analysis (a) was performed by unpaired T-test (*P<0.05, **P<0.01).

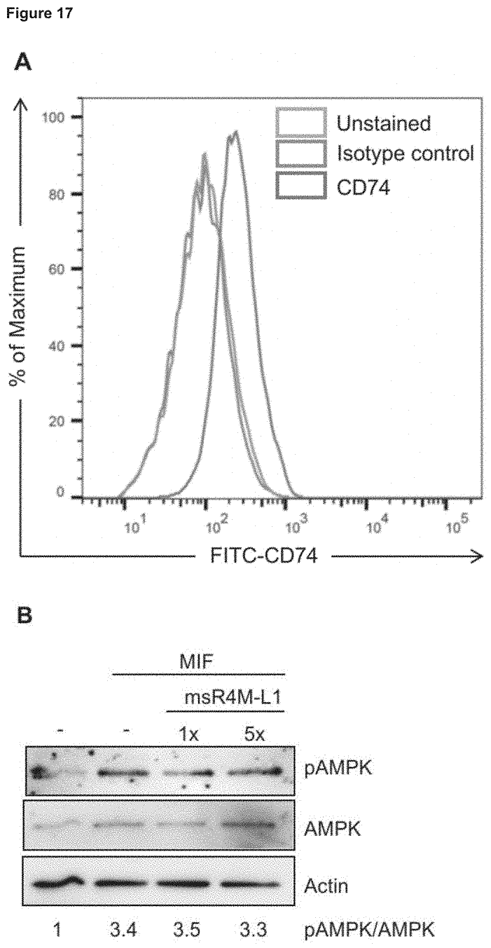

FIG. 17 msR4M-L1 does not interfere with MIF-triggered AMPK signaling in the CD74-expressing human cardiomyocyte cell line HCM. a Human cardiac myocytes (HCM) express CD74 on their surface. Flow cytometry verified marked surface expression of CD74 on HMCs. A FITC-conjugated anti-CD74 antibody (green) was used for detection. FITC-IgG2 (isotype control, blue) and cell incubations without antibody were measured as negative controls. b msR4M-L1 does not interfere with MIF-triggered AMPK signaling. MIF was applied at a concentration of 16 nM; msR4M-L1 was added at 1- and 5-fold molar excess over MIF. A representative Western blot (from a total of five independent experiments) from HCM lysates developed against pAMPK and total AMPK is shown. The blot was quantified by densitometry of pAMPK normalized against AMPK; the signal intensity expressed as pAMPK/AMPK is indicated. Actin is shown as additional loading control.

FIG. 18 Binding affinity between MIF and the small molecule MIF inhibitor ISO-1 as determined by fluorescence titration spectroscopy. Emission spectra of Alexa-MIF alone (blue) and with increasing concentrations of ISO-1 at indicated ratios are shown (left panel; representative titration); binding curve derived from the fluorescence emission at 519 nm (right panel). The derived binding affinity (K_{D}) is 14.4±4.4 μM. The data shown are means±SD of n=3 experiments.

FIG. 19 Fluos-msR4M-L1 stains atherosclerotic plaque tissue from atherogenic mice in a MIF-selective manner. Cryosections of the atherosclerotic predilection site of brachiocephalic artery (BCA) from atherogenic Mif-expressing Apoe^{-/-} (top) versus Mif-deficient Apoe^{-/-}Mif^{-/-} (bottom) on Western-type high-fat diet (HFD) were prepared for immunofluorescent staining and incubated with 500 nM Fluos-msR4M-L1. DAPI was used for counter-staining. BCA sections from Apoe^{-/-}Mif^{-/-} only show background staining, indicating MIF selectivity of the Fluos-msR4M-L1 positivity. PC, phase contrast image of tissue section; P, plaque area; L, lumen; M, media. Scale bar: 200 μm.

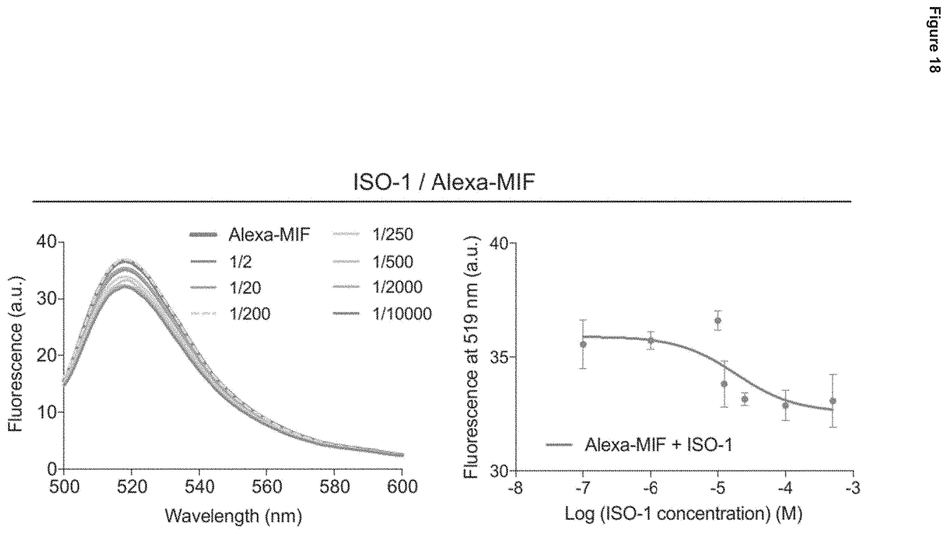

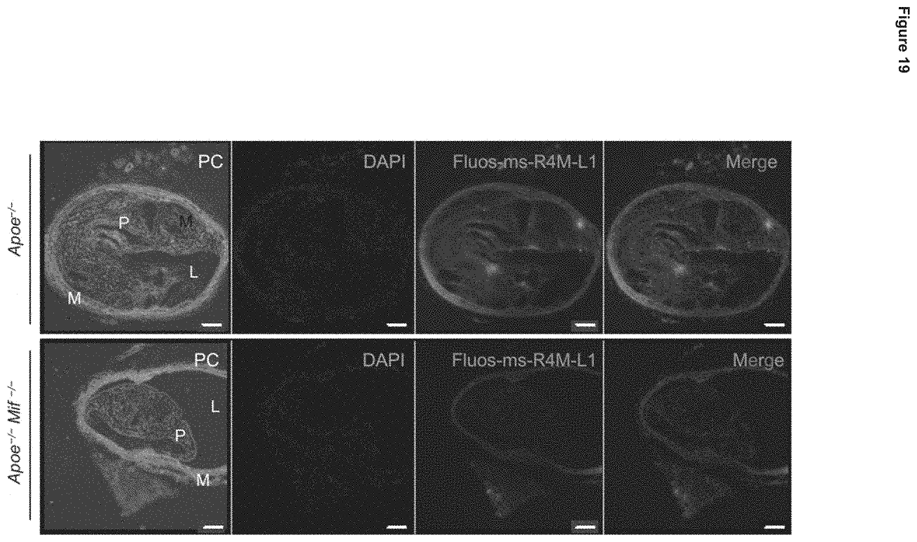

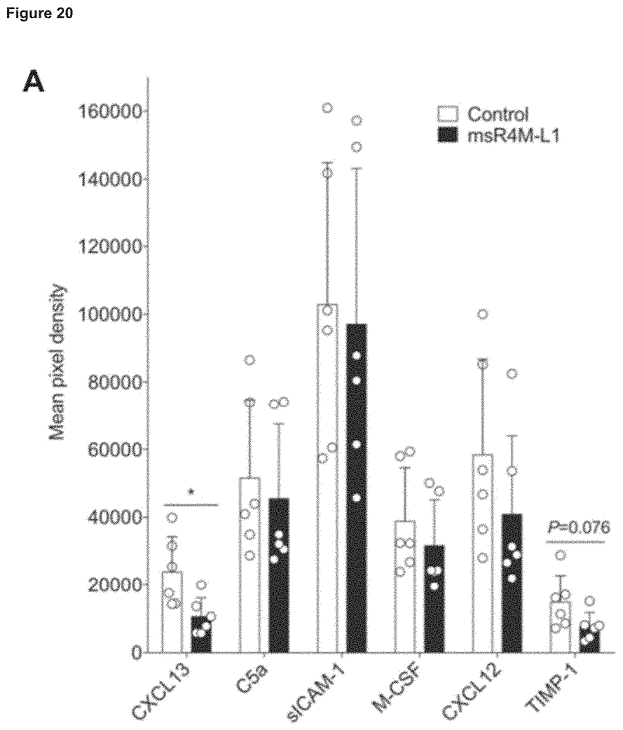

FIG. 20 Therapeutic administration of msR4M-L1 in atherogenic Apoe^{-/-} mice leads to a reduction in circulating inflammatory cytokine levels. Mouse cytokine array panel A (R&D Systems) featuring 40, mostly inflammatory/atherogenic, cytokines/chemokines was performed on plasma samples from Apoe^{-/-} mice on HFD that were therapeutically treated with msR4M-L1 or vehicle control according to FIG. 6a. The quantitative analysis of 34 of these cytokines/chemokines is shown in FIG. 6i. a Quantitative analysis of the levels of the remaining 6 cytokines/chemokines of the panel, i.e. those that have much higher circulating levels and give much higher signals on the developed array membrane. b Array set-up (top) and images of the developed arrays (bottom). The experiment shown represents the analysis of two out of six mice from each cohort (labelled 1, 2); each array analysis was performed in duplicate. Statistics are reported as means±SD from six independent experiments, corresponding to six mice per group, performed in duplicate each. Statistical analysis (a) was performed by multiple T-test or Mann-Whitney test as appropriate (*P<0.05). The P value for TIMP-1, which shows a trend towards reduction, is given. Cytokines/chemokine names are used according to the panel nomenclature. BLC, CXCL13; C5a, complement factor 5a; G-CSF, granulocyte-colony-stimulating factor; GM-CSF, granulocyte-macrophage-CSF: sICAM-1, soluble ICAM-1; IFN-γ, interferon-gamma: IL-1ra, IL-1 receptor antagonist; CCL/CXCL, CC-type and CXC-type chemokine; M-CSF, monocyte-CSF; IL, interleukin: TIMP-1, tissue inhibitor of metalloproteinases-1; TNF-α, tumor necrosis factor-a; TREM-1, soluble form of triggering receptor expressed on myeloid cells-1.

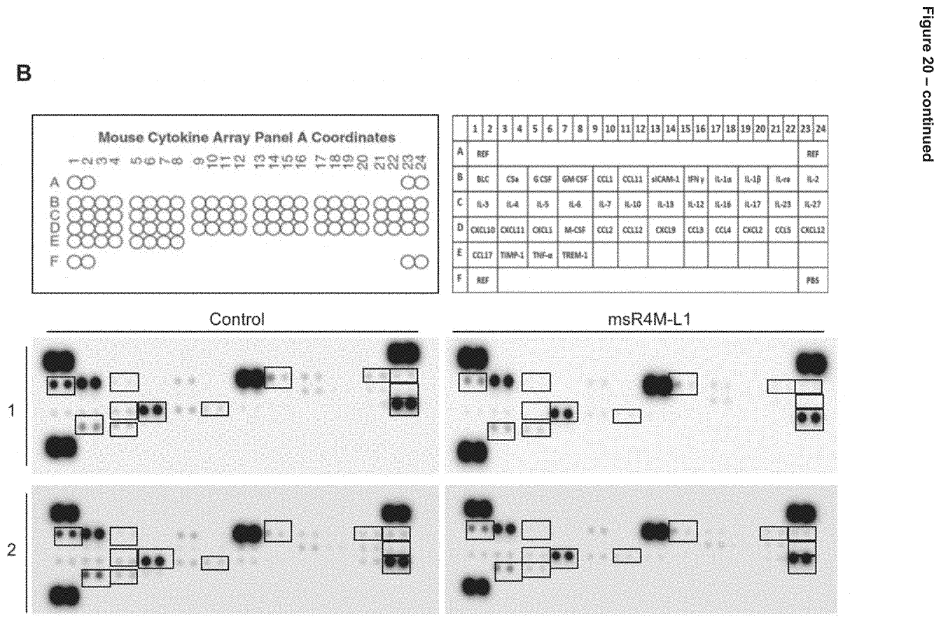

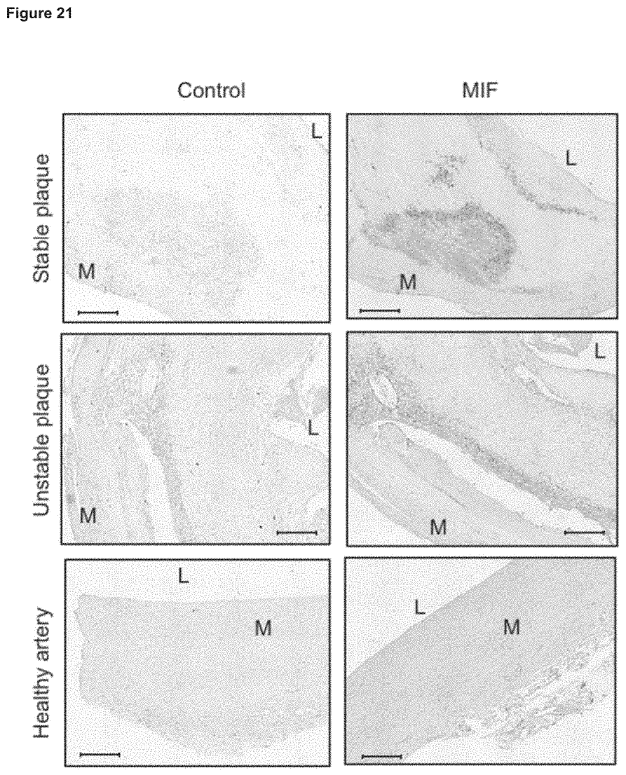

FIG. 21 MIF protein expression in human carotid atherosclerotic plaque tissue from patients who underwent carotid endarterectomy (CEA). MIF was detected by DAB-based immunohistochemistry (IHC) using a polyclonal anti-MIF antibody (MIF) and counterstaining with Mayer hematoxylin. Representative images from a stable plaque, an unstable plaque, and healthy control vasculature. Control stainings were performed in the absence of primary antibody (control). Size bars are 500 μm. Plaque/vessel regions are indicated for orientation (L, lumen; M, media). For quantification or multiple images, see FIG. 6l.

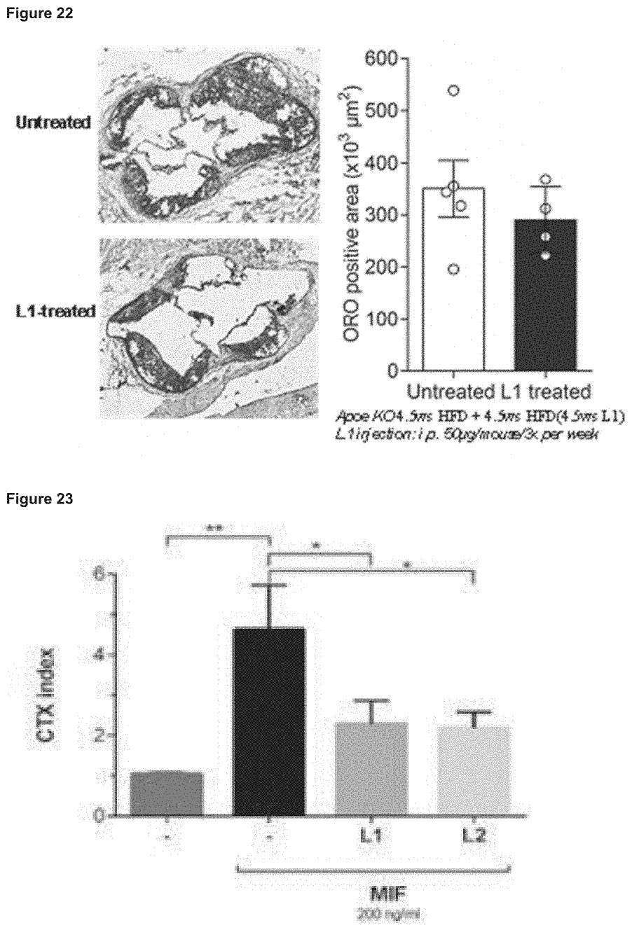

FIG. 22 Therapeutic effect of the CXCR4 mimic msR4M-L1 in a mouse model of atherosclerosis. The effect of msR4M-L1 was tested in a regression setting, where the mimic was administered after plaques had formed. Therapeutic administration of msR4M-L1 in atherogenic Apoe−/− mice with established plaques reduces plaque formation in aortic root (trend). left, Representative images of aortic roots from msR4M-L1-treated (L1-treated) versus vehicle-treated (untreated) mice. right, Quantification (4-5 mice per group) of oil red O-stained sections from msR4M-L1- versus vehicle-treated mice. Apoe−/− mice were fed a Western diet for 4.5 weeks, then msR4M-L1 treatment (50 μg per mouse, 3× per week) or vehicle was started for 4.5 weeks together with a continued Western diet.

FIG. 23 CXCR4 ectodomain mimic msR4M-L2 inhibits MIF-triggered lymphocyte chemotaxis. Chemotactic migration (Transwell) of primary mouse spleen B lymphocytes elicited by 16 nM (or 200 μg/ml) MIF as chemoattractant and inhibitory effect of msR4M-L2 (L2) compared to msR4M-L1 (L1). msR4M-L2 has a similar chemotaxis-inhibitory capacity as msR4M-L1. Data are reported as means±SD of n=3. Statistical analysis was performed with unpaired T-test (*P<0.05, **P<0.01).

FIG. 24 General architecture and modularity of GPCRs. Major regions and structural features of GPCRs are shown on an example of the dopamine receptor D3R crystal structure (PDB ID3PBL). Figure from Ref. Katritch et at, Trends Pharmacol Sci2012, doi:10.1016/j.tips.2011.

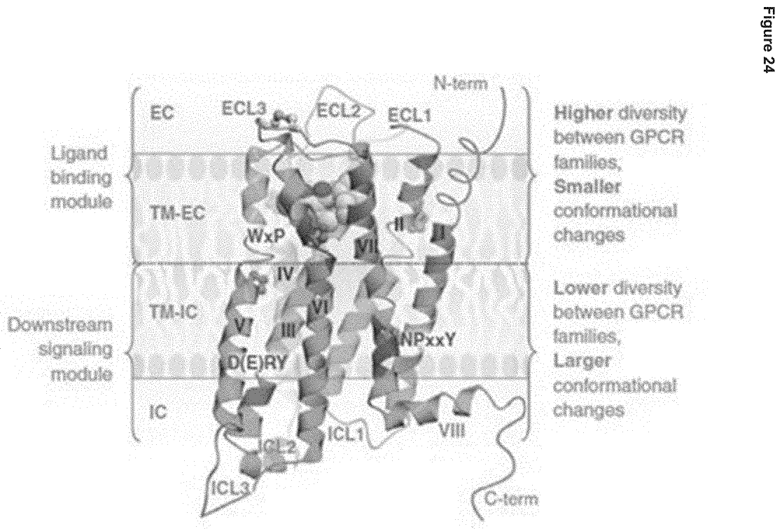

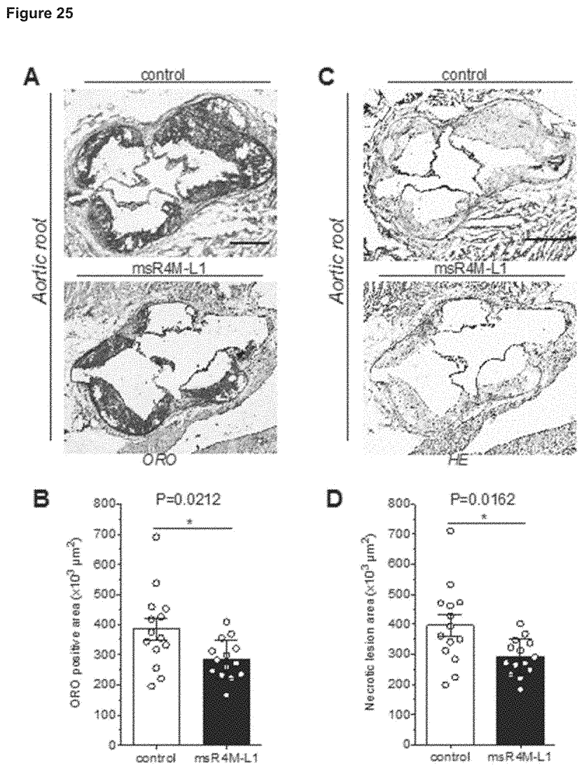

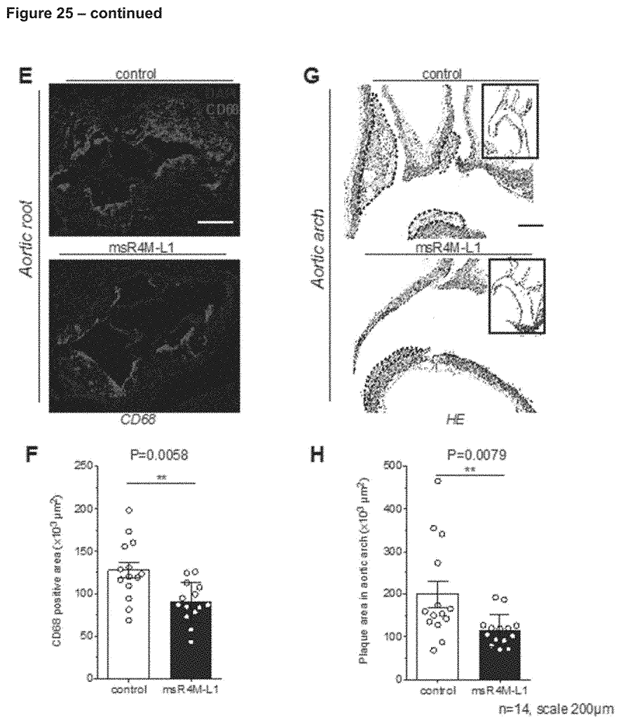

FIG. 25 msR4M-L1 reduces atherosclerotic plaques in a regression treatment regimen. Mice were fed a Western diet for 4.5 weeks; then mice were administered msR4M-L1 for the next 4.5 weeks (50 μg per injection per mouse, 3× times per week), while the Western diet was continued. Control mice received saline. Mice were sacrificed after 9 weeks and vessel tissues prepared for plaque, cell, and macrophage staining. A. C, E, staining of aortic root sections with oil red O (ORO), hematoxylin eosin (HE), and anti-CD68, respectively. G, staining of aortic arch with HE. B, D, F, H, respective quantifications from 14 mice per group. Statistics: two-tailed, unpaired Mann-Whitney test; *, P<0.05; **, P<0.01.

FIG. 26 illustration of the generation of “next generation mimics” (NGMs or ngms)

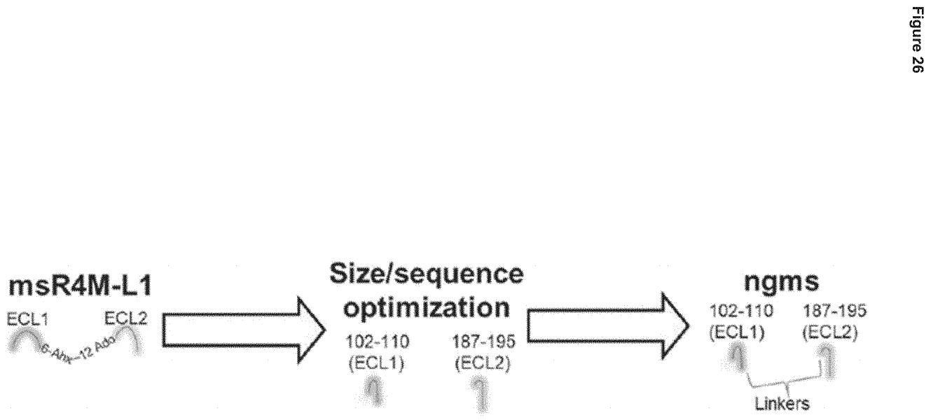

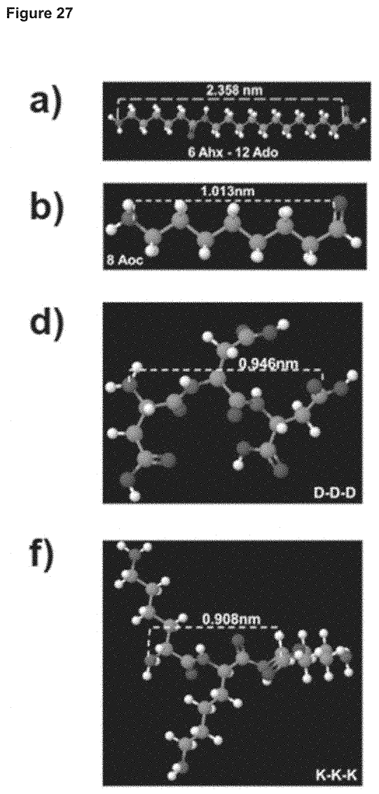

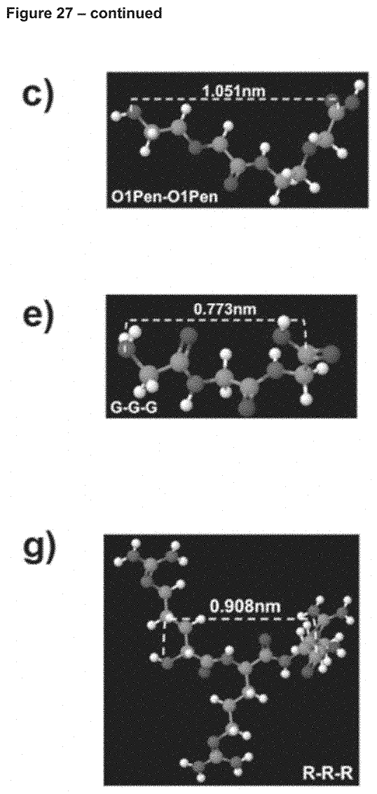

FIG. 27 Structures and dimensions of the spacers used in the NGMs. 6-Ahx-12-Ado (A) was used in msR4M-L1 and the NGM msR4M-L3. 8-Aoc (B) was used in msR4M-L4 and 01Pen-O1Pen (C) in msR4M-L5. D) shows the spacer used in msR4M-LD3, E) that used in msR4M-LG3, F) that in msR4M-LK3, and G) that in msR4M-LR3.

FIG. 28 shows examples of the HPLC chromatograms and mass spectrograms for msR4M-L5 and ms-R4M-LD3.

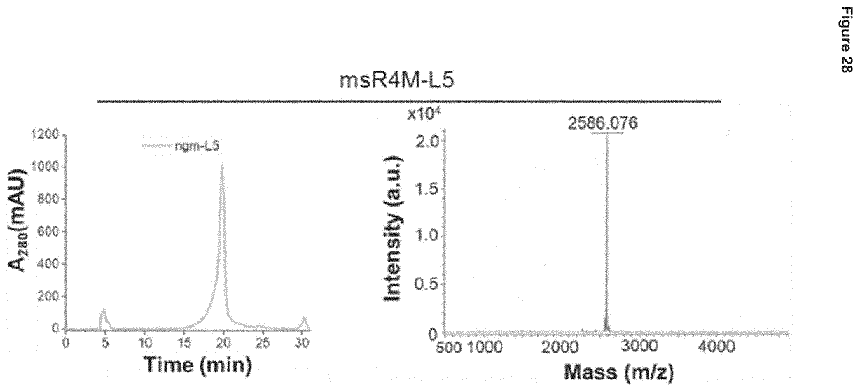

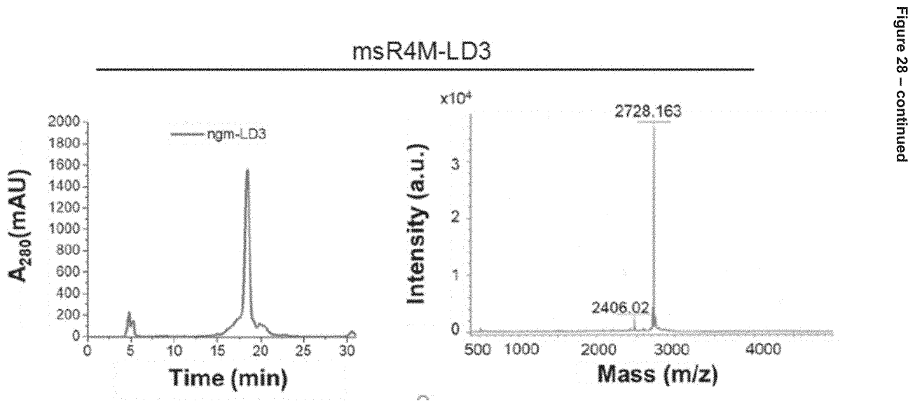

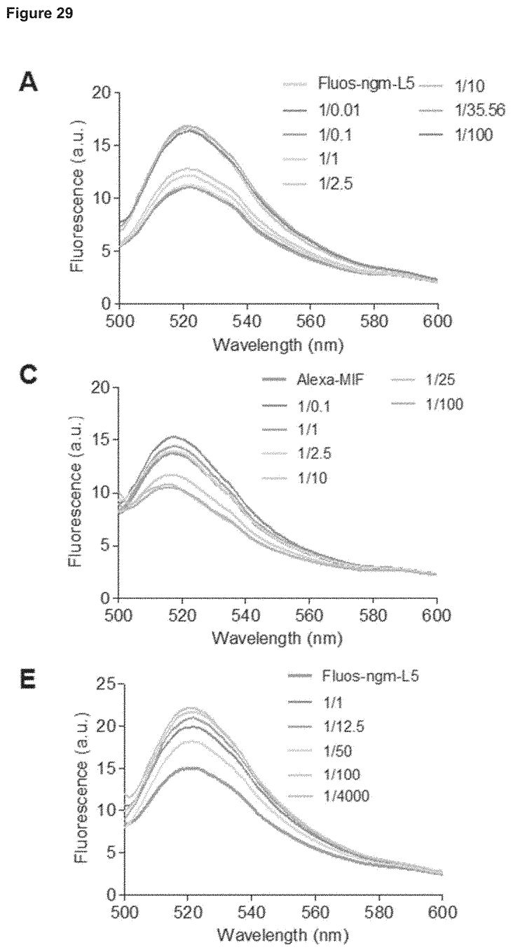

FIG. 29 Fluorescence spectroscopic titrations indicate that msR4M-L5 binds to MIF but not to CXCL12. A-B) Fluos-labelled msR4M-L5 was titrated with increasing concentrations of MIF. C-D) Alexa-labelled MIF was titrated with increasing concentrations of msR4M-L5. E-F) Fluos-labelled msR4M-L5 was titrated with increasing concentrations of CXCL12. A, C, E) Titrations curves over the entire wavelength spectrum. B, D, F) Graphs plotting different doses of MIF, msR4M-L5, and CXCL12, respectively, against the change at the peak wavelength (522 or 519 nm) according to panels A, C, E). Means+/−SD of 3 experiments are shown.

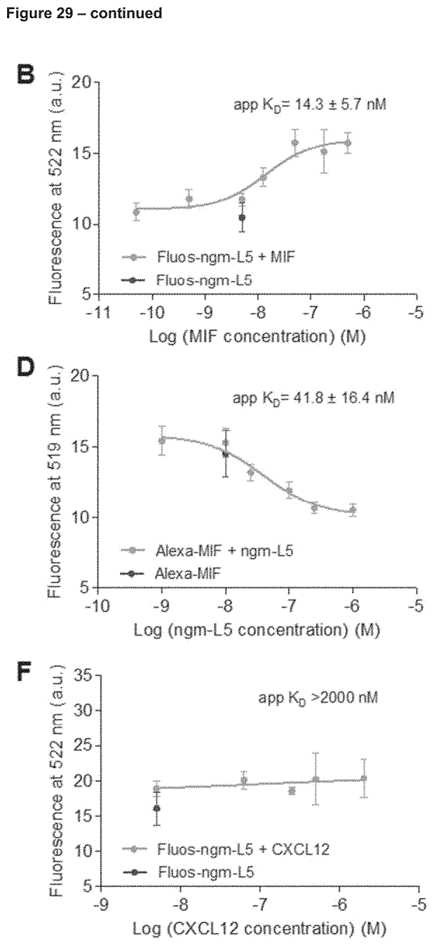

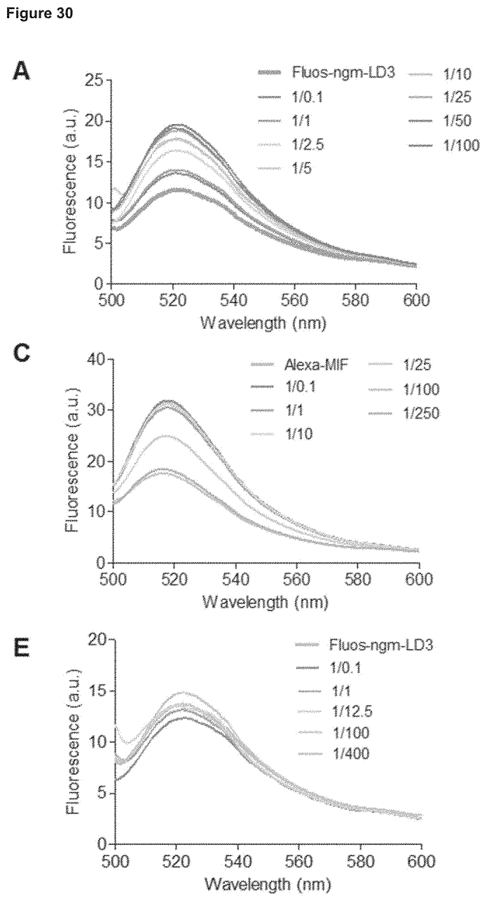

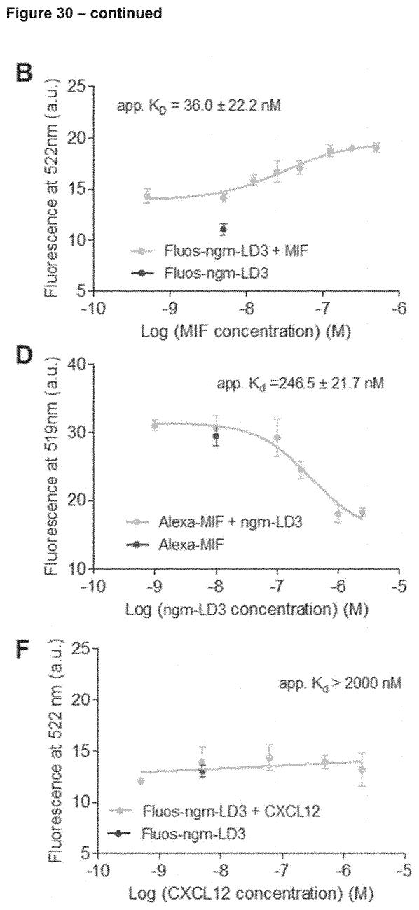

FIG. 30 Fluorescence spectroscopic titrations indicate that msR4M-LD3 binds to MIF but not to CXCL12. A-B) Fluos-labelled msR4M-LD3 was titrated with increasing concentrations of MIF. C-D) Alexa-labelled MIF was titrated with increasing concentrations of msR4M-LD3. E-F) Fluos-labelled msR4M-LD3 was titrated with increasing concentrations of CXCL12. A, C, E) Titrations curves over the entire wavelength spectrum. B. D. F) Graphs plotting different doses of MIF, msR4M-LD3, and CXCL12, respectively, against the change at the peak wavelength (522 or 519 nm) according to panels A, C, E). Means+/−SD of 3 experiments are shown.

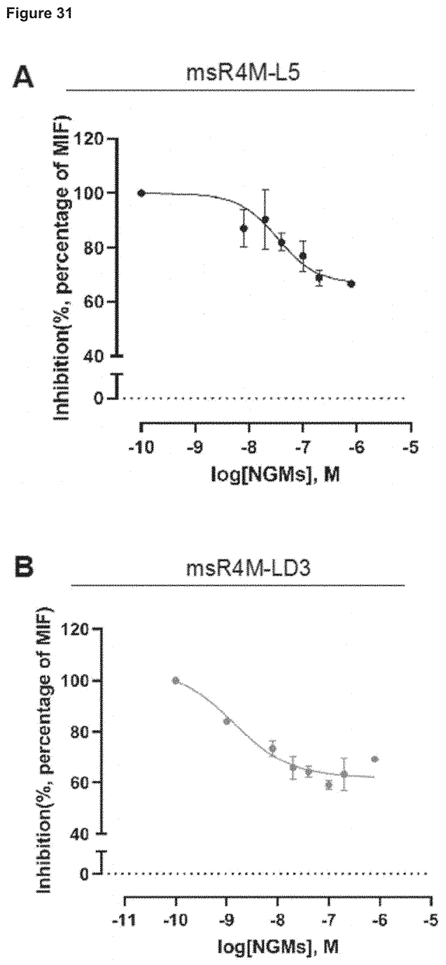

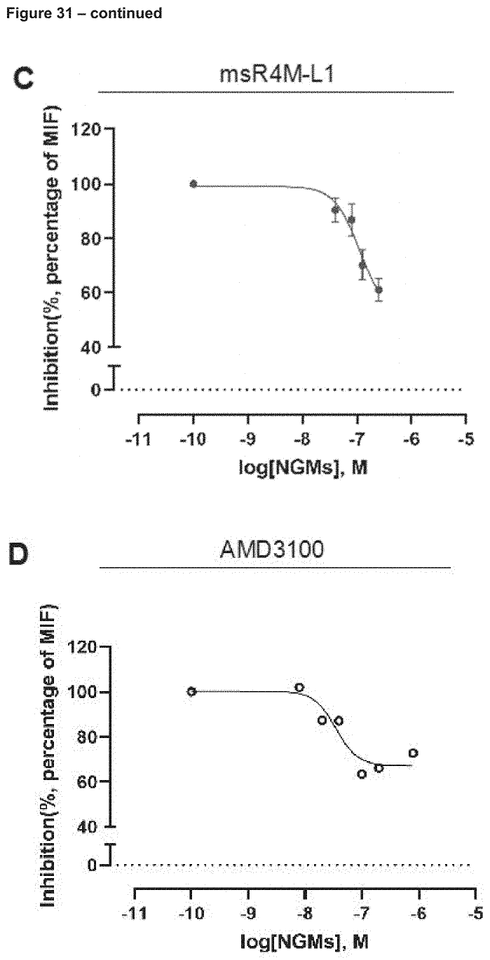

FIG. 31 msR4M-L5 and msR4M-LD3 inhibit the foam ceN-promoting activity of MIF as measured by Dil-oxLDL uptake assay in human monocyte-derived macrophages. Dose/IC50 curves of msR4M-L5 (A) and msR4M-LD3 (B). The concentration of the mimics (log scale) is plotted against relative inhibition depicted as percent of MIF-triggered Dil-oxLDL uptake, which is set at 100%. msR4M-L1 was titrated for comparison (C). The titration of the CXCR4 inhibitor AMD3100 (D) indicates that the CXCR4-dependent part of MIF-triggered oxLDL uptake accounts for approximately 50%.

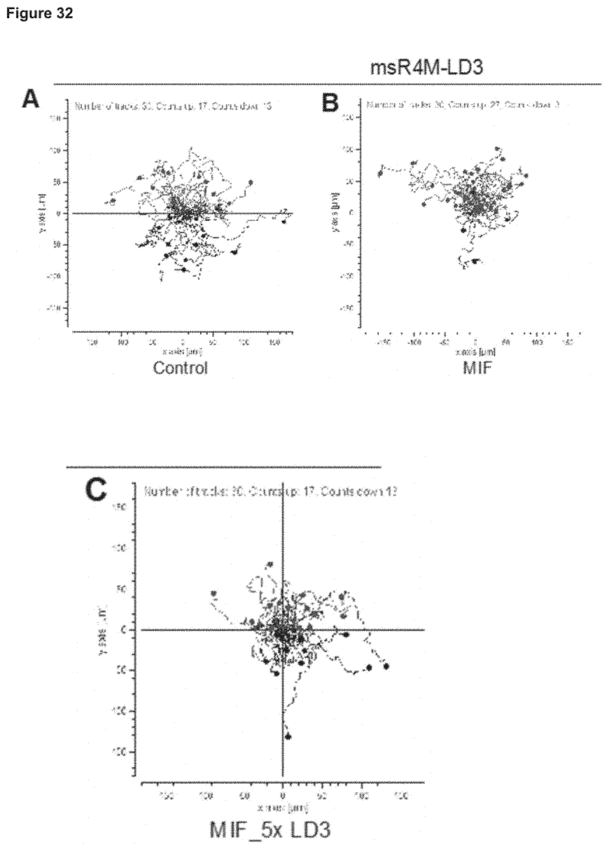

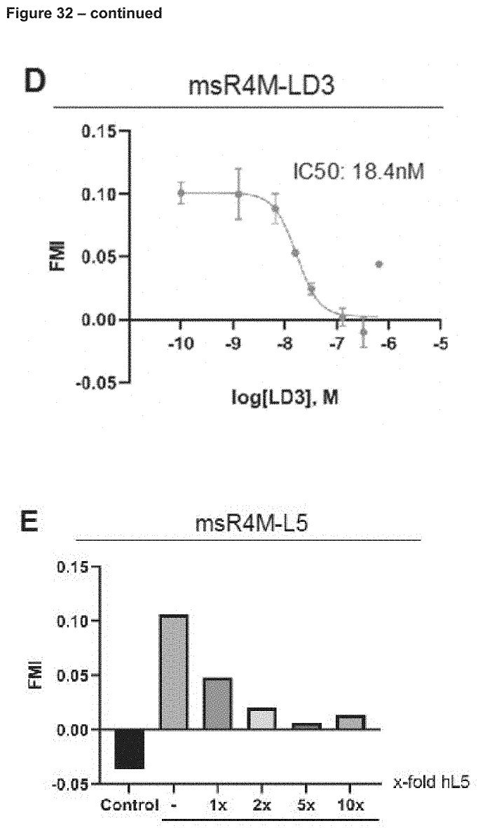

FIG. 32 msR4M-LD3 and msR4M-L5 inhibit the MIF-mediated monocytes migration as measured by single cell tracking in a 3D migration setup (Ibidi p-slides). A-C) Representative tracking experiments with 30 cells tracked each. A) Control, no chemokine added; cells randomly migrate in both directions. B) MIF added as chemokine to the upper chamber. C) Same as B) except that a 5× molar excess of msR4M-LD3 was added together with MIF. D) dose curve of msR4M-LD3 with a derived IC50 value of 18.4 nM. A dose curve for msR4M-L5 is shown in (E): data are from one experiment each (30 tracks per incubation) and the mimic is plotted as molar excess over MIF.

The Examples illustrate the invention.

### EXAMPLE 1—DESIGNING SOLUBLE CHEMOKINE-SELECTIVE CXCR4 ECTODOMAIN MIMICS

Previous peptide array and SAR studies of the inventors showed that residues 97-110 of ECL1 and 182-196 of ECL2 of the CXCR4 ectodomain contribute to the interface between MIF and CXCR4²⁵. It was therefore speculated whether this could be a basis to engineer soluble MIF-binding CXCR4 mimics. Peptides ECL1[97-110] and ECL2[182-196] were synthesized by solid-phase peptide synthesis (SPPS) using Fmoc chemistry²⁸.

A synthetic linker was chosen based on the CXCR4 X-ray structures^{28, 29, 30}. The conformationally constrained ectodomain mimic CXCR4-ECL1[97-110]-6-Ahx-12-Ado-ECL2[182-196] was designed and generated (‘msR4M-L1’; FIG. 1a,b; FIG. 14; Table 2; FIG. 15) that contained a 6-aminohexanoic acid (6-Ahx)/12-amino-dodecanoic acid (12-Ado) linker with a spacer length of 2.358 nm.

TABLE 1
Summary list and molecular masses of all synthesized CXCR4 ectodomain peptides applied
in this study.

| | Peptide | [M+H]⁺ | [M+H]⁺ |
|---|---|---|---|
| Peptide acronym | sequence | theoretical^{[a]} | experimental^{[a]} |
| ECL1 | NH₂-DAVANWYFGNFLCK-CONH₂ (SEQ | 1646.79 | 1670.02^{[b]} |
| | ID NO: 3) | | |
| ECL2 | NH₂-DRYICDRFYPNDLWW-CONH₂ | 1973.94 | 1974.38 |
| | (SEQ ID NO: 4) | | |
| mSR4M-L1 | ECL1-6-Ahx-12-Ado-ECL2 | 3912.92 | 3913.67 |
| msR4M-L2 | ECL1-O2Oc-12 Ado-ECL2 | 3944.93 | 3945.49 |
| msR4M-L1ox | 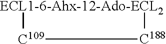 | 3910.90 | 3911.37 |
| msR4M-L2ox | 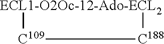 | 3942.91 | 3943.47 |
| msR4M-LS | 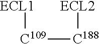 | 3618.68 | 3618.78 |
| Fluos-ECL1 | Fluos-DAVANWYFGNFLCK-CONH₂ | 2004.75 | 2027.93^{[b]} |
| Fluos-ECL2 | Fluos-DRYICDRFYPNDLWW-CONH₂ | 2330.92 | 2332.08 |
| Fluos-msR4M-L1 | Fluos-ECL1-6-Ahx-12-Ado-ECL2 | 4270.91 | 4271.72 |
| Biotin-6-Ahx- | Biotin-6-Ahx-ECL1-6-Ahx-12 Ado-ECL2 | 4252.06 | 4252.47 |
| msR4M-1L1 | | | |
| TAMRA-mSR4M-L1 | TAMRA-ECL1 -6-Ahx-12-Ado-ECL2 | 4325.06 | 4363.81^{[c]} |
| Fluos-msR4M-L2 | Fluos-ECL1-O2Oc-12 Ado-ECL2 | 4302.92 | 4303.28 |
| Fluos-msR4M-L1ox | 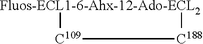 | 4268.89 | 4268.95 |
| Fluos-msR4M-L2ox | 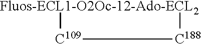 | 4300.90 | 4301.39 |
| Fluos-msR4M-LS | 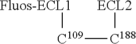 | 3976.67 | 3976.14 |

Table legend. The theoretical masses [M+H]⁺ (a) were calculated based on the monoisotopic mass
(M) and are Indicated for all peptides compared to the [M+H]⁺ (a), ([M+Na]⁺ (b), or [M+K]⁺ (c) masses
determined experimentally by MALDI-MS. ECL, extracellular loop; msR4M, MIF-specific CXCR4
ectodomain mimic; Fluos, fluorescein; TAMRA, 5-carboxy-tetramethylrhodamine; 6-Ahx, 6-
aminohexanoic acid; 12-Ado, 12-amino-dodecanoic acid; O2Oc, 3,6-dioxaoctanoic acid; NH2-,
indicates free N-terminus of respective peptide; -CONH₂, indicates C-terminal amidation of respective
peptide.

3,6-dioxaoctanoic acid (O2Oc)/12-Ado was chosen as an alternative, more hydrophilic, linker (‘msR4M-L2’; FIG. 7; Table 2; FIG. 8). The non-linked loop peptides was synthesized for comparison as well as variants of msR4M-L1 and -12 that were additionally constrained by a disulfide bridge in the presence (msR4M-L1ox and -L2ox) or absence of the synthetic linker (msR4M-Lis) (Table 2). Residues of the CXCR4 N-terminal were not include, because this region has been implicated as a critical region contributing to the CXCL12/CXCR4 interface^{29, 30, 31}, which was wished to be specifically excluded from the targeting strategy.

To determine whether the CXCR4 ectodomain mimics bind to MIF, fluorescence titration spectroscopy^{32, 33} was applied measuring changes in fluorescence emission of Fluos-labeled ectodomain peptide upon titration against MIF or CXCL12. Conversely, Alexa-Fluor 488-labeled MIF (Alexa-MIF) was titrated against unlabeled ectodomain peptides. Importantly, msR4M-L1 exhibited high affinity binding to MIF with an apparent (app.) K_{D}<40 nM (app. K_{D} Fluos-msR4M-L1/MIF=36.8±2.3 nM; app. K_{D} msR4M-L1/Alexa-MIF=31.1±16.6 nM), whereas no binding to CXCL12 was observed (app. K_{D}>5 μM) (Table 1; FIG. 1c,d; FIG. 10).

TABLE 2
Binding affinities between the CXCR4 ectodomain
peptides and MIF versus CXCL12 as determined
by fluorescence titration spectroscopy.

| | MIF or CXCL12 | | |
|---|---|---|---|
| | MIF | | |
| CXCR4 | Fluos-ECD | | CXCL12 ^{[a]} |
| ectodomain | peptide/ | Alexa-MIF/ | Fluos-ECD peptide/ |
| (ECD) | MIF ^{[b]} | ECD peptide ^{[c]} | CXCL12 |
| peptide | app. K_{D} (nM) ^{[d]} | app. K_{D} (nM) | app. K_{D} (nM) |
| msR4M-L1 | 36.8 ± 2.3 | 31.1 ± 16.6 | >5000 |
| msR4M-L2 | 18.6 ± 2.9 | 40.5 ± 7.6 | >5000 |
| msR4M-L1ox | 28.9 ± 2.5 | 30.0 ± 6.3 | 84.6 ± 42.1 |
| msR4M-L2ox | 105.3 ± 44.9 | 59.6 ± 15.3 | 54.8 ± 10.3 |
| msR4M-LS | 6.9 ± 2.0 | n.d. | 17.4 ± 4.7 |
| ECL1 | n.d. | 345.2 ± 79.4 | n.d. |
| ECL2 | >5000 | 2458 ± 1054 | n.d. |

Table legend.
^{[a]} Alexa-CXCL12 measurements were not pursued, because of the notion that Alexa labeling could interfere with the crucial residue Lys-1 of CXCL12³⁰ as well as other binding-relevant lysines.
^{[b]} Fluos-labeled ECD peptides used at a concentration of 5 nM;
^{[c]} Alexa-MIF used at 10 nM.
^{[d]} Reported apparent K_{D} values are means ± SD from three independent binding curves and were calculated as described³².
ECD, extracellular domain; app., apparent; n.d., not determined.

msR4M-L2 bound to MIF with similar affinity and also lacked CXCL12 binding (app. K_{D} Fluos-msR4M-L2/MIF=18.6±2.9 nM; app. K_{D} msR4M-L2/Alexa-MIF=40.5*7.6 nM; app. K_{D} Fluos-msR4M-L2/CXCL12 >5 μM; Table 1; FIG. 10). Thus, both mimics exhibit a >140-fold selectivity for MIF versus CXCL12. Interestingly, additional conformational restriction of the mimics by disulfide bridging led to an ‘induction’ of CXCL12 binding, while high-affinity MIF binding was preserved in such variants (Table 1: FIG. 10). By contrast, the single, non-linked ECL peptides ECL1[97-110] and ECL2[182-196] only exhibited a medium-low binding affinity for MIF (app. K_{D} ECL1/Alexa-MIF=345.2*79.4 nM; app. K_{D} ECL2/Alexa-MIF=2458*1054 nM; Table 1; FIG. 10).

These experiments suggested that msR4M-L1 and -L2 could represent promising CXCR4 mimics with high selectivity for MIF versus CXCL12. However, msR4M-L2 exhibited a higher self-assembly propensity than msR4M-L1 (app. K_{D} Fluos-msR4M-L2/msR4M-L2=69.6±61.9 nM versus Fluos-msR4M-L1/msR4M-L1=142.0*48.9 nM; FIG. 11). Of note, circular dichroism (CD) spectroscopy indicated that msR4M-L1 is well folded (FIG. 1e). CD spectroscopy also confirmed our design strategy with appreciable conformational restriction introduced by the 6-Ahx-12-Ado linker of msR4M-L1, as a non-linked mixture of ECL1 and 2 exhibited mostly random coil conformation (FIG. 12).

Thus, msR4M-L1 was selected as a lead and wished to further confirm its ligand binding selectivity. When immobilized MIF and CXCL12 were probed on a dot-blot membrane with 5(6)-carboxytetra-methylrhodamine (TAMRA)-msR4M-L1. MIF was readily detected in a concentration-dependent manner, whereas TAMRA-msR4M-L1 showed no binding to CXCL12 (FIG. 1f,g). It was also sought to validate binding between msR4M-L1 and MIF by microscale thermophoresis (MST), representing an additional solution method. MST analysis of TAMRA-msR4M-L1 and MIF overall confirmed high-affinity binding (app. K_{D} TAMRA-msR4M-L1/MIF=77.2±37.1 nM; FIG. 1h). MIF residues 80-87 and Tyr-100 are specific determinants of the interaction between MIF and CD74, but Pro-2 of MIF is not only important for MIF/CD74 binding, but also partially contributes to the MIF/CXCR4 interface^{21, 22, 25}. We therefore applied MST to experimentally confirm that msR4M-L1 does not interfere with MIF binding to its receptor CD74, which mediates MIF's cardioprotective activity¹⁵. MST titration of 50 nM Monolith NT-647 (MST-Red)-labeled MIF with increasing concentrations of HA-tagged sCD74(73-232) up to 2 μM was not influenced by 2 μM msR4M-L1 (app. K_{D} MST-Red-MIF/HA-sCD74(73-232)=54.5±36.6 nM; MST-Red-MIF/HA-sCD74(73-232)+msR4M-L1=21.8±15.4 nM; p=ns), supporting the notion that msR4M-L1 does not compete with MIF binding to CD74.

Together, the data demonstrate that msR4M-L1, an engineered soluble CXCR4 ectodomain mimic, binds with high affinity to MIF, exhibiting binding selectivity for MIF versus the cognate ligand CXCL12, while not interfering with MIF/CD74 binding. This led us to prioritize msR4M-L1 for further analysis.

### EXAMPLE 2—ENGINEERED CXCR4 MIMICS BIND TO A CORE BINDING REGION IN MIF

Next, the binding region in MIF that is necessary for interaction with the CXCR4 mimics was mapped. As previous structure-activity studies on the MIF/CXCR4 interface had provided evidence for a role of the N-like loop^{24, 25}, the mapping started with a MIF peptide fragment spanning this region (FIG. 2a). Applying fluorescence spectroscopy, MIF peptide 38-80 was found to bind to msR4M-L1 with similar affinity as full-length MIF (app. K_{D} Fluos-msR4M-L1/MIF[38-80]=57.1±7.8 nM; FIG. 2a,b; Table 3: FIG. 13).

TABLE 3
Binding affinities (K_{D}) between the CXCR4 ectodomain
peptide msR4M-L1 and partial MIF peptides as determined
by fluorescence titration spectroscopy.

| Overall screen of MIF(2-115) | | Screen of binding region MIF(38-80) | |
|---|---|---|---|
| | App. K_{D} | | App. K_{D} |
| MIF sequence | (nM) | MIF sequence | (nM) |
| 2-16 | >20000 | 38-80 | 57.1 ± 7.8 |
| 6-23 | >20000 | 38-60 | >20000 |
| 13-27 | >20000 | 38-64 | >20000 |
| 18-32 | >20000 | 38-68 | 696.3 ± 26.3 |
| 23-38 | >20000 | 38-72* | 160.7 ± 89.6 |
| 28-43 | >20000 | 38-76 | 42.2 ± 27.9 |
| 38-80 | 57.1 ± 7.8 | 50-60 | >5000 |
| 69-90 | >20000 | 50-80 | 55.2 ± 9.9 |
| 76-90 | >20000 | 51-67* | >20000 |
| 81-94 | >20000 | 54-80 | 70.6 ± 14.2 |
| 81-95 | 481.1 ± 43.5 | 55-80 | 479.4 ± 154.7 |
| 81-102 | 480.2 ± 83.1 | 56-69* | 1819 ± 491 |
| 82-95 | >10000 | 57-80 | 283.1 ± 57.7 |
| 86-100 | >20000 | 58-80 | 540.4 ± 206.3 |
| 91-105 | >10000 | 60-74* | >20000 |
| 101-115 | >20000 | 60-80 | 1758 ± 272 |
| | | 62-80 | >5000 |

Table legend.
Fluorescence spectroscopic analyses were performed as described in FIG. 2 of the main manuscript. Fluos-msR4M-L1 was generally applied at a concentration of 5 nM; the asterisk (*) denotes those peptide titrations for which Fluos-msR4M-L1 was used at 10 nM. The numbering of the sequence of human MIF (2-115) refers to the cDNA sequence and accounts for the notion that the N-terminal Met-1 residue is processed. MIF, human macrophage migration-inhibitory factor; app., apparent.

As the peptide lacks the 3D conformation of folded full-length MIF, this suggested that a locally-defined sequence was sufficient for the interaction with the ectodomain mimic. Moreover, detailed mapping of the msR4M-L1/MIF binding region by analyzing various 14-30-meric MIF peptide fragments spanning regions within and outside of sequence 38-80, narrowed the core binding region in MIF to sequence 50-80 or 54-80 (app. K_{D} Fluos-msR4M-L1/MIF[50-80]=55.2±9.9 nM; app. K_{D} Fluos-msR4M-L1/MIF[54-80]=70.6±14.2 nM; FIG. 2c; Table 3; FIG. 13). This MIF core region additionally bound to msR4M-L2 with high affinity (app. K_{D} Fluos-msR4M-L2/MIF[50-80]=30.9±20.4 nM; app. K_{D} Fluos-msR4M-L2/MIF[54-80]=52.9±25.6 nM). Together, these data suggest that the msR4M binding region of MIF is located in MIF sequence stretch 54-80, consistent with a role of the N-like loop²⁵.

Molecular docking simulations between CXCR4-ECL1 [97-110]-Gly₍₇₎-ECL2[182-196], an msR4M-L1-like CXCR4 ectodomain mimic with a heptaglycine linker instead of the 6-Ahx-12-Ado spacer, suggested that msR4M-L1 has a reasonable energetic probability of interacting with MIF and confirmed the experimentally determined binding interface within sequence 54-80, with amino acids of this region among the top residues predicted to be involved in msR4M-L1 binding (FIG. 14).

### EXAMPLE 3—CXCR4 ECTODOMAIN MIMICS SELECTIVELY INHIBIT MIF-TRIGGERED CXCR4 BINDING, SIGNALING AND CHEMOTAXIS, BUT DO NOT INTERFERE WITH CXCL12/CXCR4 AND MIF/CD74 SIGNALING

To scrutinize whether selective msR4M-L1/MIF binding correlates with inhibition of MIF-triggered inflammatory and atherogenic effects, it was first examined whether msR4M-L1 interfered with MIF/CXCR4-specific cell signaling. Advantage was taken of a yeast strain that expresses human CXCR4 and specifically measures agonist-mediated activation of CXCR4 via a reporter plasmid²⁵. Confirming previous data²⁵, MIF triggered CXCR4-mediated signaling, but co-incubation of MIF with msR4M-L1 blocked the β-galactosidase reporter signal in a concentration-dependent manner (FIG. 3a). In contrast, CXCL12/CXCR4-elicited signaling remained unaffected by msR4M-L1 (FIG. 3b). This suggested that msR4M-L1 specifically blocks MIF/CXCR4-driven cell signaling responses.

Receptor signaling analysis in the disclosed yeast system is limited to GPCRs and not amenable to the non-GPCR receptor CD74. To verify that msRM4-L1 does not interfere with the MIF/CD74 axis in a cell-based system, we transfected HEK293 cells with a construct driving CD74 surface expression¹⁴. Alexa-MIF cell surface binding as measured by flow cytometry was elevated in a CD74-dependent manner. Of note, co-incubation of Alexa-MIF with a 5-fold molar excess of msR4M-L1 did not reduce surface binding of Alexa-MIF (FIG. 3c). Together, the yeast-CXCR4 and HEK293-CD74 transfectant data showed that msR4M-L1 blocks the interaction between MIF and cell surface-expressed CXCR4, whereas binding to cell-surface CD74 is not affected.

It was next asked whether msR4M-L1 also selectively inhibits MIF responses in mammalian cell systems expressing endogenous CXCR4. B lymphocytes express substantial levels of CXCR4 and MIF has been shown to trigger murine B-cell chemotaxis in a CXCR4-dependent manner³⁴. Human and murine MIF share 90% amino acid identity and there is a high degree of cross-species receptor activity⁵. There also is a high degree of sequence identity between human and murine MIF in the binding region for msR4M-L1 (MIF(38-80): 86%; MIF(54-80): 89%; FIG. 15). By applying dot blot titration, it was confirmed that msR4M-L1 binds equally well to human and mouse MIF (FIG. 3d). Primary splenic B cells were then subjected to MIF-triggered chemotaxis using the Transwell system. When co-incubated with MIF, msR4M-L1 (as well as msR4M-L2; FIG. 16) fully blocked MIF-mediated B-cell chemotaxis in a concentration-dependent manner with maximal inhibition seen at a 5-fold molar excess (FIG. 3e) and an IC₅₀ in the range of 10-15 nM (FIG. 16). In contrast, msR4M-L1 was unable to inhibit chemotaxis elicited by CXCL12 (FIG. 3f). Thus, the yeast signaling and B-cell migration data suggested that msR4M-L1 potently and selectively interferes with MIF/CXCR4-mediated cell responses.

MIF is a pro-atherogenic cytokine, but also has context-dependent ‘local’ protective activity on cardiomyocytes^{14, 15, 16, 17}. Before further evaluating the translational potential of our findings, it was wished to exclude that msR4M-L1 interferes with protective MIF/CD74-mediated signaling in cardiomyocytes. Primary human cardiomyocytes (HCM; expressing CD74, FIG. 17) were incubated with MIF in the presence or absence of msR4M-L1. We then analyzed phosphorylated AMP kinase (pAMPK) levels, a correlate of protective MIF/CD74-mediated signaling. As demonstrated previously¹⁵, MIF upregulated pAMKP levels, but this effect was not attenuated by msR4M-L1 (FIG. 3g), confirming that the CXCR4 mimic does not cross-affect MIF activities through CD74.

### EXAMPLE 4—CXCR4 ECTODOMAIN MIMIC INHIBITS PRO-ATHEROGENIC MIF ACTIVITIES IN VITRO AND IN THE VASCULATURE EX VIVO

MIF is a driver of atherogenic monocyte activity and inhibition of monocyte-dependent atherogenic inflammation is a preferred strategy to limit atherosclerotic lesion formation. Monocyte/macrophage-expressed CXCR4 promotes atherogenesis via low density lipoprotein (LDL) uptake and foam cell formation, an effect specifically mediated by the MIF/CXCR4 axis but not by CXCL12/CXCR⁴⁶.

Confirming previous findings³⁶, uptake of fluorescently labeled LDL (Dil-LDL) by human macrophages derived from peripheral blood mononuclear cells (PBMCs) was markedly enhanced by MIF and this activity was blocked inhibitor by the pharmacological inhibitor AMD3100, verifying CXCR4 dependency. Of note, msR4M-L1 dose-dependently inhibited MIF-mediated Dil-LDL uptake (FIG. 4a,b). This assay also appeared suitable to compare the inhibitory capacity of msR4M-L1 with that of established MIF inhibitors. i.e. the neutralizing anti-MIF monoclonal antibody (mAb) NIH/IIID.9 and the small molecule inhibitor ISO-1. The inhibitory capacity of msR4M-L1 was slightly better than that of ISO-1, but lower than that or NIH/IIID.9 (FIG. 4b,c). This notion was confirmed by comparing the binding affinity between msR4M-L1 and MIF with those of ISO-1 and anti-MIF mAbs. Neutralizing mAbs such as NIH/IIID.9 or BAX01 bind human or mouse MIF with a K_{D} of 1-2 nM^{16, 37}; binding is thus more affine, albeit within a comparable nanomolar range, than that between MIF and msR4M-L1 (<40 nM). The K_{D} for the MIF/ISO-1 interaction has not been reported, but the IC₅₀ value for MIF/CD74 binding is 10 μM^{16, 38}. Using fluorescence spectroscopic titration, we determined the K_{D} between ISO-1 and MIF to be 14.4±4.4 μM (FIG. 18). Thus, while msR4M-L1 is superior to ISO-1 and NIH/IIID.9 in being receptor-selective, its inhibitory capacity and binding affinity is between that of small molecule inhibitors and anti-MIF mAbs.

It was next tested the potency of msR4M-L1 towards MIF-elicited three-dimensional (3D) chemotaxis of PBMCs. We applied 3D-chemotaxis methodology and assessed single-cell migration tracks via time-lapse microscopy. msR4M-L1 dose-dependently attenuated MIF-triggered motility of human monocytes as quantified by forward migration index. The pro-migratory effect of MIF was already ablated by a 2-fold molar excess of msR4M-L1 (FIG. 4d,e). By contrast, the CXCL12-induced cell motility response remained unaffected (FIG. 4f,g).

A major atherogenic process promoted by MIF is its effect on leukocyte adhesion in the atherosclerotic vasculature, an activity involving engagement of CXCR4¹⁴. To determine whether this function of MIF can be attenuated by CXCR4 mimics. MIF-triggered adhesion of MonoMac-6 monocytes on human aortic endothelial (HAoEC) monolayers under static conditions was assessed in the presence or absence of msR4M-L1. FIG. 5a shows that msR4M-L1 ablated the pro-adhesion effect of MIF. Before studying this effect in more pathogenically relevant ex-vivo and in-vivo settings, we wished to determine whether msR4M-L1 localizes to atherosderotic plaque tissue. We stained plaque sections obtained from aortic root and brachiocephalic artery of atherogenic Ldlr^{-/-} and Apoe^{-/-} mice, respectively, with Fluos-msR4M-L1 to detect plaque targeting of the CXCR4 mimic. Fluos-msR4M-L1 positivity was significantly more pronounced in plaque tissue from Mif-proficient atherogenic Ldlr^{-/-} or Apoe^{-/-} mice when compared to sections from Mif-deficient Ldlr^{-/-} Mif^{-/-} (FIG. 5b,c) or Apoe^{-/-} Mif^{-/-} (FIG. 19) mice, respectively. This showed that Fluos-msR4M-L1, similar to anti-MIF antibody, was capable of binding to plaque-associated MIF. It was next tested whether also in-vivo-administered Fluos-msR4M-L1 would localize to atherosclerotic plaque. Three days before vessel preparation, we intraperitoneally injected Fluos-msR4M-L1 into atherogenic Apoe^{-/-} mice. Multiphoton laser-scanning microscopy (MPM) analysis of whole-mount carotid arteries from these mice visualized by second harmonic generation (SHG) and fluorescein detection revealed that Fluos-msR4M-L1 strongly localized to intimal plaque areas, while staining in spleen, liver, and brain was marginal, suggesting that the CXCR4 mimic, at least partially, is targeted to atherosclerotic plaques in vivo (FIG. 5d).

To determine the functional consequence of this finding, leukocyte recruitment was studied in ex-vivo-mounted atherogenic carotid arteries using MPM. This involved injection of mice with msR4M-L1 three days before vessel preparation and visualization of in-situ adhering msR4M-L1- versus vehicle-exposed fluorescently labeled bone marrow-derived leukocytes in the vasculature under physiological flow conditions (FIG. 5e). In fact, the mimic significantly reduced the number of adhering leukocytes (FIG. 5f-h).

Together, these findings suggested that msR4M-L1 localizes to atherosclerotic plaque tissue in a MIF-specific manner and inhibits MIF-mediated atherogenic leukocyte recruitment by interfering with chemotactic migration and arterial adhesion.

### EXAMPLE 5—THE ENGINEERED CXCR4 MIMIC REDUCES ATHEROSCLEROSIS AND INFLAMMATION IN VIVO AND MARKS STABLE HUMAN CAROTID ATHEROSCLEROTIC PLAQUE TISSUE

Peptides are sensitive to proteolysis by plasma proteases and clearance. Thus, before testing the potential therapeutic utility of msR4M-L1 in vivo, its proteolytic stability was examined. Biotin-msR4M-L1 was incubated with human plasma isolated from the blood of healthy donors for various time intervals. SDS-PAGE/Western blot analysis revealed that appreciable amounts of intact, undigested biotin-msR4M-L1 could be recovered up to 16 h of plasma exposure, indicating that this peptide was reasonably stable in plasma (FIG. 6a).

To examine the therapeutic capacity of the CXCR4 mimic, an established in-vivo mouse model of early atherosclerosis was employed, in which lesions develop in aortic root and arch over a 4-5-week time course of HFD³⁹. Apoe^{-/-} mice received msR4M-L1 (50 μg per mouse i.p., three times per week) or vehicle treatment in parallel to HFD for 4.5 weeks (FIG. 6b). We did not observe any effect of msR4M-L1 administration on body weight, plasma total cholesterol, triglyceride levels, or blood leukocyte counts (Table 4).

TABLE 4
Therapeutic treatment of atherogenic Apoe^{-/-} mice
with msR4M-L1 does not affect blood leukocytes and lipid
levels. Blood cell count, body weight and serum lipid levels from
mice Apoe^{-/-} mice on cholesterol-rich high-fat diet
(HFD) for 4.5 weeks and treated with msR4M-L1 or vehicle control.

| | | |
|---|---|---|
| Vehicle | msR4M-L1 | P value |

Serum lipid levels

| | | | |
|---|---|---|---|
| Cholesterol (mg/dL) | 693.4 ± 25.2 | 647.4 ± 19.2 | 0.1686 |
| Triglycerides (mg/dL) | 161.3 ± 5.7 | 156.7 ± 3.8 | 0.5203 |

Blood cell counts

| | | | |
|---|---|---|---|
| Leukocytes (per μL) | 4874 ± 673 | 4632 ± 286 | 0.7490 |
| Monocytes (per μL) | 702 ± 128 | 552 ± 51 | 0.3096 |
| Lymphocytes (per μL) | 3070 ± 560 | 2347 ± 50 | 0.2343 |
| Neutrophils (per μL) | 2115 ± 272 | 1897 ± 46 | 0.4538 |

Body weight

| | | | |
|---|---|---|---|
| Weight (g) | 22.8 ± 0.4 | 22.3 ± 0.2 | 0.2848 |

Table legend.
Shown are means ± SD. P-values calculated by Student’s t-test. N = 5 (blood cell counts), n = 7 (body weight), and n = 11 (lipids) per group.

Importantly, atherosclerotic lesion size in aortic arch (FIG. 6c,d) and root (FIG. 6e,f) was significantly decreased in msR4M-L1-treated mice compared with vehicle-treated controls. Moreover, protection from lesion formation was accompanied by a significantly decreased number of lesional macrophages in the msR4M-L1 group, as revealed by MAC-2 staining (FIG. 6g,h), and by a marked reduction in circulating inflammatory cytokine levels, as measured by a cytokine array (FIG. 6i, FIG. 20). Reductions in the msR4M-L1-treated group were seen for IFN-γ, IL-1α, IL-16, TNF-αn (FIG. 6i) as well as CXCL13/BLC (FIG. 20), with trends observed for IL-27, CCL12, and TIMP-1, indicating that the CXCR4 mimic broadly down-regulates the inflammatory response associated with atherogenesis. Together, this demonstrated that msR4M-L1 exhibits a therapeutic atheroprotective and anti-inflammatory capacity in an experimental in-vivo model of atherosclerosis.

To further test the translational relevance of these findings, stable and unstable human carotid atherosclerotic plaque sections obtained from patients undergoing carotid endarterectomy (CEA) (Table 5) were probed with Fluos-msR4M-L1.

TABLE 5
Characteristics of the atherosclerotic patients undergoing carotid endarterectomy (CEA).

| | Staining with Fluos-msR4M-L1 | | | Staining with anti-MIF | | |
|---|---|---|---|---|---|---|
| | Stable | Unstable | | Stable | Unstable | |
| | ( n = 9) | (n = 15) | P value² | (n = 11) | (n = 15) | P value² |
| Age (y)¹ | 66.1 ± 2.0 | 71.2 ± 12.6 | 0.178 | 66.6 ± 1.8 | 68.9 ± 2.7 | 0.524 |
| Sex (male, %) | 33.3 | 60.0 | 0.223 | 54.6 | 40.0 | 0.482 |
| Neurological | 22.2 | 33.3 | 0.582 | 9.1 | 26.7 | 0.280 |
| symptoms (%) | | | | | | |
| Hypertension (%) | 77.8 | 80.0 | 0.902 | 81.8 | 93.3 | 0.384 |
| Diabetes mellitus (%) | 44.4 | 20.0 | 0.219 | 45.5 | 20.0 | 0.178 |
| Hyperlipidemia (%) | 66.7 | 66.7 | 0.999 | 63.6 | 60.0 | 0.858 |
| Smoking (%) | 66.7 | 40.0 | 0.223 | 45.5 | 46.7 | 0.954 |
| CKD¹ (%) | 0 | 6.7 | 0.451 | 0 | 6.7 | 0.403 |
| Coronary heart | 0 | 6.7 | 0.451 | 9.1 | 6.7 | 0.827 |
| disease (%) | | | | | | |
| PAD² (%) | 0 | 6.7 | 0.451 | 0 | 13.3 | 0.223 |
| Aspirin/Clopidogrel | 100.0 | 93.3 | 0.451 | 100.0 | 85.7³ | 0.207 |
| (%) | | | | | | |
| Beta-blocker (%) | 22.2 | 33.3 | 0.582 | 27.3 | 28.6³ | 0.946 |
| ACE-inhibitors³ (%) | 22.2 | 13.3 | 0.591 | 18.2 | 28.6 | 0.565 |
| Statins (%) | 88.9 | 86.7 | 0.880 | 90.9 | 85.7³ | 0.706 |
| Diuretics (%) | 0 | 6.7 | 0.451 | 18.2 | 14.3³ | 0.802 |

Table legend. All atherosclerotic carotid tissue samples used for analysis showed an advanced stage of atherosclerosis (types V-VII according to the American Heart Association (AHA) guidelines).
Healthy controls were age-matched (57.3 ± 5.5 years). P values refer to stable versus unstable samples (unpaired t-test). Information regarding this parameter missing for one patient. Abbreviations: CKD, chronic kidney disease; ACE, angiotensin-converting enzyme; PAD, peripheral artery disease.

Stainings were compared with sections from healthy vessels and counter-staining against MIF was performed using an anti-MIF antibody. Based on histological characterization of plaque morphology, a total of 11 stable and 17 unstable carotid plaques were examined; 6 healthy vessels served as controls. Fluos-msR4M-L1 led to a pronounced staining of stable carotid plaque tissue that was higher than Fluos-msR4M-L1 positivity detected in unstable plaques and healthy vessels (FIG. 6j,k). Of note, the staining profile mirrored that of MIF as detected by conventional antibody-based immunostaining (FIG. 6l; FIG. 21). In contrast, as described previously. CXCL12 exhibits more pronounced expression in unstable plaque⁴⁰. Thus, these findings revealed an association with the MIF staining pattern and a functional correlation with different types of plaque stages.

### EXAMPLE 6—DISCUSSION

Anti-cytokine/-chemokine strategies represent promising therapeutic approaches for a variety of diseases, including cancer, inflammation, and cardiovascular diseases. In addition to SMDs and antibodies, soluble receptors are an important targeting approach to block pathogenic cytokine effects^{7, 41}. While soluble cytokine receptors have been developed for single-membrane spanning receptors and are successfully used in the clinic against immune-mediated diseases, anti-chemokine strategies based on a soluble receptor principle are not established.

Herein a small engineered peptide-based, soluble chemokine receptor mimic is provided that distinguishes between two chemokines and features ligand- and receptor-selective anti-atherosclerotic capacities in vitro and in vivo. We focused on CXCR4, one of the most studied chemokine receptors^{42, 43}. CXCR4 has critical ligand- and context-dependent roles in various diseases. Together with its ligand CXCL12, it is a promising target in tumor metastasis⁴² and small molecule CXCR4 inhibitors such as Plerixafor/AMD3100 are used as stem cell mobilizers for transplantation therapy of patients with specific cancers⁴⁴. However, in atherosclerotic diseases, the CXCR4/CXCL12 axis has proven to be a difficult target, with both disease-promoting and protective properties. Genome-wide association studies (GWAS) and CXCL12 plasma level analysis revealed CXCL12 as a candidate gene associated with CAD^{45, 46, 47}, and disease-exacerbating activities such as cardiac inflammatory cell recruitment have been implied for the CXCR4/CXCL12 axis^{48, 49}. In contrast, beneficial activities include cardioprotective effects based on the contribution of CXCR4/CXCL12 to neoangiogenesis and cardiomyocyte survival^{43, 50, 51}. Moreover, disruption of this axis promotes atherosclerotic lesion formation through deranged neutrophil homeostasis⁵² and loss of atheroprotection²⁶. In this context, we have shown that atherogenesis-induced endothelial damage is counter-acted by unleashing CXCR4 activity and autocrine CXCL12 expression in endothelial cells through miR-126-containing apoptotic bodies²⁷ and that CXCR4 on vascular cells maintains arterial integrity and limits atherosclerosis by preserving barrier function and a normal contractile vascular smooth muscle cell (VSMC) phenotype²⁶.

Capitalizing on our earlier findings that CXCR4 engages MIF as a non-cognate ligand to drive atherogenic leukocyte recruitment^{14, 16, 17} and that CXCR4-supported endothelial barrier integrity is mediated by CXCL12 but not MIF²⁶, we surmised that MIF-specific CXCR4 targeting might be a promising avenue to circumvent the complexity of the CXCR4/CXCL12 system in cardiovascular conditions. In fact, we previously demonstrated that MIF-blocking strategies are superior to CXCL12 blockade in inducing plaque regression^{6, 14} and that the foam cell-promoting activity of CXCR4 is primarily elicited by MIF and not CXCL12³⁶. However, currently available MIF blocking strategies may not be optimal, as anti-MIF (Imalumab) or anti-CD74 (Milatuzumab) antibodies would potentially interfere with the cardioprotective MIF/CD74 axis^{15, 16}. The same holds true for MIF-directed SMDs, which are designed to bind in MIF's conserved tautomerase pocket and interfere with MIF binding to CD74. However, modification of this cavity invokes conformational changes in MIF that impair binding to CD74⁵³. AMD3100 partially interferes with MIF/CXCR4 binding^{14, 25}, but this CXCR4 inhibitor has been found to impair the cardio- and atheroprotective activity spectrum of the CXCR4/CXCL12 axis^{26, 27, 52}.

The disclosed engineering design was guided by the CXCR4 structures^{29, 30, 31} and SAR studies^{24, 25}, highlighting CXCR4 ectodomain regions that may be harnessed to engineer a soluble receptor mimic to selectively target MIF and spare CXCL12. Approaches to utilize the ectodomains of single membrane-spanning type I cytokine receptors such as the TNF or IL-6 receptor have been successfully developed as immunomodulatory drugs^{7, 41}. However, mimicking the ectodomain of seven-helix membrane-spanning GPCRs is inherently complex due to the discontinuous nature of the receptor backbone topology. Ligand binding in (poly)peptide-ligating GPCRs such as chemokine receptors typically involves several extracellular portions of the receptor, often a combination of residues of several ECLs and the N-terminal³⁰. Only a handful of reports are available: the N-terminal and ECL3 elements of CXCR1 and CXCR2 were assembled on a soluble GPCR B1 domain scaffold protein⁵⁴; based on the crystal structure of rhodopsin, all three predicted ECLs of CXCR4 were connected to form an HIV gp120-binding mimic⁵⁵; and a construct mimicking corticotropin-releasing factor receptor-1 (CRF-R1) combined native chemical ligation and recombinant technology and encompassed the entire 23 kDa ectodomain of CRF-R1⁵⁶. Such studies have remained explorative, led to constructs with micromolar binding affinities, and neither chemokine selectivity, nor in-vivo or disease relevance were addressed.

The engineered MIF-selective CXCR4 mimics reported here are highly reduced GPCR mimics of only 29 residues plus two non-natural amino acids of the linker moiety (molecular weight <4 kDa), reducing the size of CXCR4 by >90%. MIF selectivity over CXCL12 was achieved by combining only selected residues within ECL1 and ECL2. As determined by independent biophysical methods, the lead candidate mimics bind MIF with low nanomolar affinity (K_{D}˜30 nM), in line with the reported K_{D} value of 19 nM for MIF binding to full-length membrane CXCR4¹⁴, while binding to CXCL12 is essentially absent. This affinity is reasonable compared with that of Imalumab or the pre-clinical anti-MIF antibody NIH/III.D9 (K_{D}˜1-3 nM)⁵⁷ and MIF-directed SMDs (micromolar K_{D})¹⁶. Of note, despite its high affinity, msR4M-L1 neither affected MIF binding to CD74, nor did it impair MIF/CD74-mediated stimulation of AMPK phosphorylation in human cardiomyocytes as a correlate of MIF's cardioprotective activity¹⁵. Hence, msR4M-L1 has more favorable selectivity characteristics than the available anti-MIF therapeutic strategies. A molecular explanation for this selectivity comes from experiments mapping the MIF binding site, msR4M-L1 targets MIF region 54-80, a part of the N-like loop known to mediate MIF/CXCR4 binding, but not involved in MIF/CD74 binding, in line with data showing that the tautomerase site of MIF and residues 80-87 determine the MIF/CD74 binding interface^{21, 22, 55}. Importantly, binding selectivity of msR4M-L1 for MIF versus CXCL12 was functionally paralleled in a number of inflammation- and atherosclerosis-relevant cell systems, i.e. GPCR/CXCR4 signaling, 2D lymphocyte chemotaxis, foam cell formation, monocyte adhesion, and 3D monocyte migration, representing MIF/CXCR4-mediated cell systems with disease relevance³⁶.

Intriguing structural information also comes from mimics, in which we introduced a disulfide bridge between residues Cys-109 of ECL1 and Cys-186 of ECL2. In contrast to msR4M-L1 and -12 that are fully selective for MIF, introduction of the disulfide bridge led to a gain-of-CXCL12-binding activity, irrespective of the presence (msR4M-L1ox, msR4M-L2ox) or absence (msR4M-LS) of the spacer-mediated conformational constraint. This is in line with the identification of a Cys-109-Cys-188 disulfide in the X-ray structure of CXCR4^{29, 30} and structural insights on the CXCR4/CXCL12 interface⁵⁸, and supports the notion that the natural CXCR4 receptor is ‘equipped’ to interact with both CXCL12 and MIF¹⁴. On the other hand, the K_{D} for MIF binding dropped >10-fold, when the respective ECL1 and 2 sequences were not covalently linked. Together, these data indicate that the MIF binding-determining sequence elements within the CXCR4 mimics need to be covalently linked, but that conformational restriction needs to allow for a certain flexibility to guarantee selectivity between different CXCR4 chemokines. Comparison of the various synthesized mimics further instructs for future optimization towards higher potency, stability, or selectivity²⁸.

The biochemical and cell-based experiments encouraged us to examine whether the mimics would be efficacious in a pathogenic ex-vivo organ or in-vivo setting. Using fluorescently labeled msR4M-L1 to stain atherosclerotic tissue sections from atherogenic mice and in-vivo-administration of this peptide verified that msR4M-L1 localizes to and marks atherosclerotic plaque tissue in a MIF-specific manner. Indeed. MIF has been shown to be upregulated in atherosclerotic lesions, where secreted MIF is deposited similar to classical arrest chemokines and localizes to plaque macrophages, foam cells, and VSMCs^{14, 59, 60}. While these experiments do not fully exclude the possibility that msR4M-L1 also—partially—localizes to CXCL12+ regions, our biochemical data proving binding selectivity, suggest that this is unlikely. Furthermore, while the MIF homolog MIF-2/D-DT¹³ has not been studied in atherosclerosis, it may be of future interest to design mimics directed at MIF-2 for applications in MIF-2-dominated inflammatory conditions.

An MPM-based ex-vivo atherosclerotic carotid artery system was used to monitor luminal leukocyte adhesion under pathophysiologically relevant conditions and demonstrated that treatment with msR4M-L1 markedly attenuated adhering leukocyte numbers. Such systems have been powerful in demonstrating the leukocyte recruitment potential of MIF or classical arrest chemokines such CXCL1/KC^{14, 39, 52, 61, 62}. In conjunction with the Fluos-msR4M-L1 plaque staining data, the MPM data indicate that msR4M-L1 blocks MIF-mediated atherogenic leukocyte recruitment. Important proof for a translational utility of the GPCR mimics reported here comes from testing msR4M-L1 therapeutically in a mouse model of atherosclerosis in vivo³⁹. The chosen treatment regimen of three 50 μg-injections per week maintained circulating doses of the mimic in line with the determined K_{D}/IC₅₀ values. The mimic potently blocked atherosclerosis at key predilection sites, reduced lesional macrophage accumulation and circulating inflammatory cytokines/chemokines, while no effects on lipids or leukocyte counts were observed, suggesting that it specifically targeted a MIF-mediated pathogenic inflammatory effect in atherogenic lesions. The experiment constitutes a ‘proof-of-concept’ for such compounds in an in-vivo disease setting and is a good predictor for their efficacy in advanced atherosclerosis models, but also other models involving MIF-related chronic inflammation^{12, 14, 16, 17, 52, 63}. In fact, a pilot study indicates a beneficial role of msR4M-L1 in a 9-week regression type of atherosclerosis model, although the data currently only suggests a trend and did not reach statistical significance (FIG. 22). Moreover, CXCR4 is a major receptor driving cancer metastasis, and not only the CXCR4/CXCL12 but also the CXCR4/MIF axis has been implicated in this process^{4, 64}. While it is beyond the scope of this study to address the inhibitory potential of our peptides in cancer, the mimics appear principally suitable for such an application. Further, the selectivity differences seen between our covalently linked conformationally restricted versus non-linked versus hyper-restricted constructs may instruct for the design of dual-specificity inhibitors against both MIF and CXCL12, e.g. for future applications in cancer. Similarly, it may be envisaged to expand the concept to MIF/CXCR2, which also has a role in atherosclerosis^{1, 14}.

The CANTOS trial has provided clinical proof that an immunotherapy-based targeting approach against IL-1α, a key inflammatory mediator, improves cardiovascular outcome in an at-risk population^{4, 65}. However, treatment with Canakinumab did not improve mortality in atherosclerotic patients and caused an increase in infections, highlighting the need to identify additional drug targets and to develop anti-inflammatory strategies with a high selectivity profile that block atherosclerotic pathways. Engineering of CXCR4 mimics towards MIF specificity could be one such approach and represent a novel class of anti-atherogenic molecules based on the soluble GPCR ectodomain concept. MsR4Ms are peptide-based molecules and, while there are over 60 peptide drugs approved worldwide, there are pros and cons compared to antibodies and SMDs. Advantages are a good surface coverage and hence high selectivity and potency, favorable safety, and low-cost production; disadvantages are the limited proteolytic stability and bioavailability⁶⁶. However, these issues can be overcome by peptide chemistry tools and peptide design strategies^{28, 66}. Thus, msR4M-L1 should be viewed as a proof-of-concept inhibitor of MIF/CXCR4-specific atherogenesis, whose properties may be improved by designed second-generation mimics. Accordingly, studies in patients with atherosclerotic disease could be a future perspective. In fact, staining of human carotid artery samples from patients who underwent CEA with Fluos-msR4M-L1 revealed interesting clinical correlations with stable versus unstable plaque phenotypes that mirrored the MIF expression profile in these lesions. In accord, CXCL12 expression was previously found to be more prominently expressed in unstable plaque tissue⁴⁰.

In conclusion, the designed MIF-selective soluble CXCR4 mimics are a novel class of anti-atherosclerotic/-inflammatory agents that could complement currently available inhibition strategies by antibodies or SMDs. It is demonstrated that these molecules can be engineered to be chemokine-selective, to exhibit high binding affinities, and to be potent in blocking atherogenic chemokine activities in vitro and in vivo, while sparing potentially contraindicative protective pathways through alternative receptors or ligands.

### EXAMPLE 7—NEXT GENERATION MIMETICS

There are over 60 peptide drugs approved worldwide, but there are pros and cons compared to antibodies and SMDs. Advantages of peptide drugs are a good surface coverage and hence high selectivity and potency, favorable safety, and low-cost production; disadvantages are the limited proteolytic stability and bioavailability⁶⁶. The disadvantages can be overcome by peptide chemistry tools and peptide design strategies^{28, 66}. To this end, one typical approach pursued in the field is to shorten the bioactive peptide sequence, to identify required and dispensable residues, and to translate this information into the design of shorter, more stable peptide analogs that retain activity, and to design peptidomimetics. The second-generation mimics described herein are representative of such an approach and represent shorter peptides themselves with retained full activity.

On the basis of “msR4M-L1”

97 110 182 196
NH2-DAVANWYFGNFLCK-6-Ahx-12-Ado-DRYICDRFYPNDLWV-
CONH2

residues and sequence positions in msR4M-L1 that are dispensable and are available for substitutions to introduce D-amino acids, non-natural amino acids, N-methylated amino acids, and amino acids that may be used for covalent cyclization were identified. Identification relied on various analyses, e.g. a sequence comparison between the presumed binding sites of CXCR4 for MIF versus CXCL12, on data from peptide arrays and from an alanine-scanning approach, and on data from a shortening approach of the msR4M-L1 sequence (“fragment approach”).

This led to the identification of the following dispensable residues: D182, R183, I185, C186, R188, V196.

These are available for the above described substitutions towards more stable and more active second-generation mimics and also represent candidate residues for a shortening approach.

Depending on their role in peptide conformation, the substitution of individual residues in a peptide sequence also can lead to an “increase” in inhibitory activity of the peptide. Such residues have an intrinsic inhibitory activity and dampen the effect. The substitution of such “inhibitory” residues can lead to peptide variants with higher, “improved” binding activity. In the present invention, potential “Inhibitory” residues were identified by substitution with Ala. Accordingly, Ala substitutions in the following positions led to an increase in binding affinity to MIF compared to the parent sequence in msR4M-L1 or a respective shorter sequence: D97, A98, V99, (A100), L108, C109, K110, P191, D193 This analysis also led to the identification of shorter fragments of msR4M-L1 with partial MIF binding and partial inhibitory activity that can serve as a basis or scaffold for short next generation mimics.

Their binding activity as determined by fluorescence spectroscopic binding assay is summarized in Table 5.

Table 6: MIF binding activity of shortened ECL1 and ECL2 fragments of msR4M-L1
fragment 100-110: Kd=215+/−72 nM
fragment 101-110: Kd=51 nM
fragment 102-110: Kd=80+/−8 nM
fragment 187-195: Kd=286+/−35 nM
fragment 185-195: Kd=324+/−30 nM
(comp. msR4M-L1: Kd=30-35 nM)

Their MIF binding potential is further confirmed in the Dil-LDL uptake-based assay, a surrogate assay representing atherogenic foam cell formation. The fragments of Table 5 exhibit a ca. 80% inhibitory capacity compared to full-length msR4M-L1 in the Dil-LDL foam cell assay.

Next, the shortened ECL fragments with partial MIF-binding/inhibitory activity were reconnected in an attempt to generate shortened msRM4 variants comprising the minimally required residues from both the ECL1 and ECL2 loop sequences. Table 6 summarizes the reconnected short “active” fragments:

TABLE 7
Reconnected short mimics msR4M-L3, -L4, and -L5:

| | | | Atherogenic |
|---|---|---|---|
| | | Binding affinity | inhibitory |
| | Sequence & linker | (fluorescence | activity |
| | (ECL1 - | spectroscopic | (Dil-LDL |
| Name | linker - ECL2) | titration) | uptake) |
| msR4M-L3 | 102-110-6-Ahx-12- | Kd = 14 +/− 4 nM | 80% of |
| | Ado-187-195 | | full-length |
| | | | msR4M-L1 |
| msR4M-L4 | 102-110-8-Aoc-187- | Kd = 12 +/− 5 nM | 80% of |
| | 195 | | full-length |
| | | | msR4M-L1 |
| msR4M-L5 | 102-110-O1-Pen-O1- | Kd = 14 +/− 6 nM | 30% of |
| | Pen-187-195 | | full-length |
| | | | msR4M-L1 |

As a next step, reconnected short mimics of -L1 with improved solubility properties were created by introducing solubility-enhancing linkers and tags, which introduce positive or negative charges. This was achieved by introducing three lysine residues (K3), three arginine residues (R3), three aspartic acid residues (D3), or three glycine residues (G3) as N-terminal tag, C-terminal tag, or as linker between the shortened ECL1 and 2 sequences. As Table 7 shows, the introduction of these solubility-enhancing residues in msR4M-G3, -D3, -R3, and -K3 retained the high binding affinity to MIF as determined by fluorescence spectroscopic binding assay and by CD spectroscopy:

TABLE 8
Reconnected short mimics with enhanced solubility:

| | Sequence & | Binding affinity | Estimated |
|---|---|---|---|
| | linker | (fluorescence | solubility |
| | (ECL1 - | spectroscopic | index by |
| Name | linker - ECL2) | titration) | CD spectroscopy |
| msR4M-G3 | 102-110-G-G-G- | Kd = 35 +/− 6 nM | >2× compared to |
| | 187-195 | | msR4M-L1 |
| msR4M-D3 | 102-110-D-D-D- | Kd = 33 +/− 21 nM | >2× compared to |
| | 187-195 | | msR4M-L1 |
| msR4M-R3 | 102-110-R-R-R- | 22 +/− 2 nM | >2× compared to |
| | 187-195 | | msR4M-L1 |
| msR4M-L5 | 102-110-K-K-K- | ca. 30-40 nM | >2× compared to |
| | 187-195 | | msR4M-L1 |

Furthermore, second-generation mimics will feature advantageous properties such as improved proteolytic stability by introducing conformational constraints via lactam-bridge- or disulfide-mediated cyclization, while accounting for the required conformational flexibility as determined from the comparison of the structure-activity relationships between msR4M-L1 and -L2 with -L1ox, -LS, and L2ox (see Table 1).

### EXAMPLE 8—THERAPEUTIC APPLICABILITY OF MSR4M-L1 IN A REGRESSION MODEL

To further test the therapeutic applicability of msR4M-L1, an in vivo test in a “regression setting” was applied to mimic the patient situation, who is typically seen by a physician only when symptoms occur. The real-life situation is thus one in which preformed plaques already exist, when a patient starts treatment. A regression model therefore better mimics the situation in patient with pre-existing atherosclerotic disease.

In a pilot study, atherogenic ApoE−/− mice were put on Western diet for 4.5 wks. Next, treatment with msR4M-L1 was performed (50 μg/mouse, 3× per week; 4-5 mice per group) in parallel with another 4.5 wks of Western diet. msR4M-L1-treated mice show a decreased plaque load (trend).

The study was extended to 14 mice per group and the data indicate that msR4M-L1 leads to a regression of atherosclerotic plaques as measured by ORO staining in aortic root, HE staining in aortic root, and HE staining in aortic arch. Intralesional inflammation as measured by CD68+ macrophage area was also reduced in the msR4M-L1 group. The effect was not as pronounced as in the early atherogenesis co-treatment model, but significant (FIG. 26)

### EXAMPLE 9—INHIBITORY CAPACITY OF MSR4M-L2 IN LEUKOCYTE CHEMOTAXIS ASSAY

msR4M-L2 (Table 2) has a similar binding affinity to MIF as msR4M-L1 (see Table 1), although it contains a spacer with different hydrophobicity. Analysis of msR4M-L2 in the MIF-elicited leukocyte chemotaxis assay showed that it has a similar inhibitory capacity as msR4M-L1 in controlling leukocyte recruitment (FIG. 23).

### EXAMPLE 10—APPLICATION TO OTHER GPCRS

Chemokine receptor and many other GPCRs display an overall similar structural architecture with a discontinuous extracellular domain (ECD) consisting of an N-domain and three extracellular loops (FIG. 24). While there are ligand-dependent differences in the conformations of ECDs from different chemokine receptors/GPCRs, the general structural principals are conserved (FIG. 24).

Accordingly, the msR4M principle can be applied to other CXC or CC chemokine receptors, or other GPCRs. Moreover, the principle can be applied to hybrid receptors combining ECD regions from different receptors to tailor, enhance, or restrict ligand binding and inhibitory specificities. For example, a msRxM hybrid between CXCR4 and CXCR2 can inhibit atherogenic functions of MIF that are mediated by both CXCR4 and CXCR2.

### EXAMPLE 11—NEXT GENERATION MIMETICS II

msR4Ms are ectodomain mimics of CXCR4, with their size varying from 3.9 to 4.3 kDa. Full-length msR4Ms such msR4M-L1 and -L2 consist of a 14-meric ECL1 and a 15-meric ECL2 covalently bonded by a non-natural linker; in msR4M-L1ox and -L2ox an additional disulfide bridge is introduced. Size optimization studies of the individual ECL1 and ECL2 loops by alanine scanning suggested that the 9-mers ECL1(102-110) and ECL2(187-195) are the shortest individual binders of MIF with a reasonable binding affinity to MIF. These fragments were then linked to shorter “next generation mimics” (NGMs or ngms) as summarized in FIG. 26.

The linkers were chosen as follows starting our considerations from msR4M-L1:

6-Ahx and 12-Ado formed the linker in msR4M-L1. Even though the determined length of the 6-Ahx-12-Ado linker is longer than the measured distance of ECL1(102-110) and ECL2(187-195) (according to the crystal structure data), a 6-Ahx-12-Ado linker was chosen to generate the next generation mimic msR4M-L3 or shortly L3. In next trying to imitate it the length of 0.95 nm between K110 and D187, the mono-unit spacer 8-Aoc and the tandem spacer O1Pen-O1Pen were introduced resulting in NGMs L4 and LS, respectively. To generate the NGMs LD3, LK3, and LR3 with predicted improved solubility properties three aspartic acid, three lysine or three arginine residues, respectively, were introduced as linker. LG3 with three glycine residues form synthesized for further comparison. FIG. 27 shows structures and dimensions of the corresponding spacers.

Table 9 summarizes the names, sequences, mass spec analysis, and apparent affinities (app. Kds) of interaction between NGMs and MIF, as determined by fluorescence spectroscopic titrations

| | | | | Fluos- | Alexa-488- |
|---|---|---|---|---|---|
| | | | | ngm/MIF | MIF/ngm |
| | Peptide | [M + H]⁺ | [M + H]⁺ | app. K_{d} | app. K_{d} |
| Peptide sequence^{[a]} | abbreviation | expected^{[b]} | found^{[b]} | (±SD) (nM) ^{[c]} | (±SD) (nM) ^{[c]} |
| [ECL1(102-110)] -6 Ahx- | ngm-L3 | 2693.36 | 2693.54 | 44.4 (±16.4) | 11.7 (±7.3) |
| 12 Ado- [ECL2(187-195)] | | | | | |
| [ECL1(102-110)] - 8 Aoc- | ngm-L4 | 2524.24 | 2524.38 | 11.9 (±4.8) | 43.2 (±20.2) |
| [ECL2(187-195)] | | | | | |
| [ECL1(102-110)]- O1pen- | ngm-L5 | 2585.23 | 2586.08 | 14.3 (±5.7) | 41.8 (±16.4) |
| O1pen - [ECL2(187-195)] | | | | | |
| [ECL1(102-110)] - D-D-D - | ngm-LD3 | 2728.22 | 2728.16 | 36.0 (±22.2) | 246.5 (±21.7) |
| [ECL2(187-195)] | | | | | |
| [ECL1(102-110)] - G-G-G - | ngm-LG3 | 2554.20 | 2555.23 | 35.0 (±19.6) | >5000 |
| [ECL2(187-195)] | | | | | |
| [ECL1(102-110)] - K-K-K - | ngm-LK3 | 2767.42 | 2767.26 | 36.4 (±7.5) | 44.8 (±10.3) |
| [ECL2(187-195)] | | | | | |
| [ECL1(102-110)] - R-R-R - | ngm-LR3 | 2851.44 | 2851.82 | 16.8 (±6.2) | 110.1 (±28.1) |
| [ECL2(187-195)] | | | | | |

Peptides were dissolved and analyzed by MALDI-TOF-MS;
^{[a]}Peptides were synthesized with free amino-N-terminal and amidated C-terminal;
^{[b]}monoisotopic molar mass with an additional hydrogen [M + H]⁺;
^{[c]} App. K_{ds}, are means (±SD) from three independent titration experiments which were performed in aqueous 1 × b, pH 7.4, containing 1% HFIP.

FIG. 28 shows examples of the HPLC chromatograms and mass spectrograms for msR4M-L5 and ms-R4M-LD3:

The binding affinity of the NGMs for MIF and that for CXCL12 were tested for comparison, to determine their affinity and selectivity for MIF. msR4M-L5 and msR4M-LD3 have high affinities for MIF but essentially no binding propensity for CXCL12 (see binding curves in FIG. 29, 30, and Table 9). They also have favorable biophysical properties such as solubility, with msR4M-LD3 performing most superior here.

msR4M-L3 and -L4 also showed high affinity for MIF (Table 9) and had essentially no binding affinity for CXCL12, but had less favorable solubility properties. msR4M-LK3 and -LR3 have very good solubility properties and also bound to MIF with high affinity (Table 9), but were also found to have good binding affinity for CXCL12.

Overall, these binding and biophysical data therefore suggested that msR4M-L5 (containing a non-natural linker moiety) and msR4M-LD3 (containing a natural triple-Asp spacer conveying very good solubility properties to the mimic) were the most favorable mimics in terms of their binding affinity for MIF, their selectivity for MIF over CXCL12, and good biophysical properties. These were therefore tested for MIF-blocking activity in prototypical atherogenic assays.

The inhibitory potential of the mimics on the atherogenic activity of MIF was tested in an oxLDL-based foam cell assay, in which the MIF-triggered uptake of Dil-labelled oxLDL is measured by microscopic quantification. The inhibitory effect of msR4M-L5 and msR4M-LD3 were tested in comparison with msR4M-L1 (FIG. 31). AMD3100 was used as a control to define the lower threshold value for CXCR4-dependent effects in this assay.

Using a lower threshold for the effect size of the MIF/CXCR4 response in this assay of roughly 50% as related to the effect of AMD3100 (IC50 of 48.9 nM), IC50 values or 69.7 nM and 1.4 nM were determined for msR4M-L5 and msR4M-LD3, respectively, which compares well with that estimated for msR4M-L1 (although the dose curve was not fully titrated) of ca. 100 nM.

In a second type of atherogenic assay, the inhibitory capacity of msR4M-L5 and msR4M-LD3 was tested on MIF-triggered monocyte migration in a 3D migration setting (FIG. 32).

### EXAMPLE 12—METHODS

Cytokines/chemokines and reagents. Biologically active recombinant MIF was prepared as reported previously and exhibited a purity of ˜98%^{14, 35}. For some of the biophysical methods, a 90-95% purified preparation was used. Fluorescently-labeled MIF¹⁸ and was generated using the Microscale Protein Labeling Kit from Invitrogen-Molecular Probes (Karlsruhe, Germany: Alexa-488-MIF) or Monolith Kit RED-NHS from NanoTemper (Munich, Germany: MST-Red-MIF). LPS content was tested by limulus amoebocyte assay (LAL, Lonza, Cologne, Germany) and verified to be <5 pg/μg. Cell culture-grade tumor necrosis factor (TNF)-α was purchased from Life Technologies (Carlsbad, United States). Recombinant CXCL12, prepared as described⁶², was a gift of Dr. von Hundelshausen (LMU Munich) or was purchased from Peprotech (Hamburg, Germany). Other reagents were obtained from Sigma, Merck, Roth, or Calbiochem, and were of the highest purity degree available.

Design, peptide synthesis, purification, and linker chemistry. Based on the crystal structures of human CXCR4 (codes 3ODU, 3OEU0, 3OE6, 3OE8, 3OE9, 4RWS) and previous SAR studies^{24, 25}, CXCR4 ectodomain peptides were selected. The crystal structures were imported into PyMOL Molecular Graphics System (Version 1.8.2.2 Schrödinger, LLC) and Jmol (http://www.jmol.org) for determining the C-to-N distance between residues 97-110 and 182-196^{29, 30}. Conjugates of 12-Ado with either 6-Ahx or O2Oc were visualized in three-dimensional space using Molview and Jmol as. The estimated distances between the N- and C-terminal in both conjugates were similar to the ECL1-ECL2 distance. All CXCR4-derived peptides were synthesized as C-terminal amides on Rink amide MBHA resin by SPPS using Fmoc chemistry as described²⁸. Couplings of Fmoc-6-Ahx-OH, Fmoc-12-Ado-OH and Fmoc-O2Oc-OH (Iris Biotech GmbH, Marktredwitz, Germany) were carried out with 3-fold molar excess of 2-(7-Aza-1H-benzotriazole-1-yl)-1,1,3,3-tetramethyluronium hexafluorophosphate (HATU) and 4.5-fold molar excess of N,N-diisopropylethylamine (DIEA) in N,N-dimethylformamide (DMF). Fmoc-deprotection was carried out with 0.1 M hydroxybenzotiazole (HOBt) in 20% v/v piperidine in dimethylformamide (DMF) for 3 and 9 min to avoid aspartimide formation³², 5(6)-carboxy-fluorescein (Fluos)- and biotin-labeled ectodomain peptides were synthesized as described³², 5(6)-carboxytetramethylrhodamine (TAMRA, Novabiochem/Merck KGaA, Darmstadt, Germany) was coupled N-terminally to side chain-protected msR4M-L1 on solid phase, after Fmoc-deprotection. Disulfide bridges in msR4M-L1ox and msR4M-L2ox were formed in 1 mg/mL peptide solution in aqueous 3 M guanidinium hydrochloride (GdnHCl) in 0.1 M ammonium carbonate (NH₄HCO₃) solution, containing 40% dimethylsulfoxide (DMSO). msR4M-LS was produced similarly, using 0.3 mg/mL ECL1 and 0.5 mg/mL ECL2 and 20% DMSO. Reverse-phase high-performance liquid chromatography (RP-HPLC) was applied for the purification of crude and oxidized peptides by using Reprosil Gold 200 C18 (250×8 mm) or Reprospher 100 C18-DE (250×8 mm) columns with pre-column (30×8 mm) (Dr. Maisch-GmbH, Herrenberg, Germany). The mobile phase consisted of 0.058% (v/v) trifluoroacetic acid (TFA) in water (buffer A) and 0.05% (v/v) trifluoroacetic acid in 90% (v/v) acetonitrile and water (buffer B) (flow rate 2.0 mL/min). All peptides were purified with an elution program of 10% B for 1 min, followed by a gradient from 10% to 90% B over 30 min, except for msR4M-LS, which was eluted with 30% B for 7 min followed by an increase to 60% B over 30 min. Expected molecular weights were verified by matrix-assisted laser desorption/ionization mass spectrometry (MALDI-MS)²⁸. Peptides were used as TFA salts. For in-vivo experiments, the TFA anion was exchanged to chloride by four cycles of dissolution/lyophilization of pure msR4M-L1 in aqueous 5 mM HCl and one cycle of bidistilled water²⁸. MIF sequence-based peptides (Table 3) were synthesized on Wang resin or purchased from Peptide Specialities GmbH (PSL, Heidelberg, Germany). MIF-derived peptides were N-terminally acetylated and had a free carboxylate function.

Fluorescence spectroscopy. Fluorescence spectroscopic titrations were performed as described^{28, 32}. Fluorescence spectra were recorded using a JASCO FP-6500 fluorescence spectrophotometer. MIF or CXCL12 were reconstituted in 20 mM sodium phosphate buffer, pH 7.2: peptide stocks were freshly made in HFIP at 4° C. as described^{28, 32}. After mixing Fluos-labeled peptides or Alexa-MIF with their unlabeled titration partner in assay buffer, measurements were performed in 10 mM sodium phosphate buffer, pH 7.4, containing 1% HFIP. Fluos-labeled peptide was applied at 5 nM and Alexa-MIF at 10 nM unless indicated otherwise. For the titration with ISO-1, Alexa-MIF had a concentration of 50 nM and ISO-1 varied from 0.1 to 500 μM in 10 mM sodium phosphate buffer, pH 7.4, containing 0.5% DMSO. The excitation wavelength was 492 nm and emission spectra were obtained between 500 and 600 nm. Apparent K_{D} values (app. K_{D}) were calculated assuming a 1/1 binding model³².

Circular dichroism (CD) spectroscopy. CD spectra were obtained with a JASCO J-715 spectro-polarimeter (JASCO, Tokyo, Japan) applying an established protocol⁶⁷. Far-UV CD measurements were carried out between 195 and 250 nm. The response time was set at 1 s, intervals at 0.1 nm, and bandwidth at 1 nm. All spectra were measured at RT and represent an average of three recorded spectra. Scans were recorded for the ectodomain mimic peptides at a concentration of 1-20 μM in 10 mM sodium phosphate buffer, pH 7.4, containing 1% HFIP, following dilution of freshly made peptide stock solution in HFIP (4° C.) into the buffer-containing cuvette. Singular ECL1 and ECL2 peptides were measured at 5 μM. The background spectrum of buffer/1% HFIP alone was subtracted from the spectra of the peptides. Dynode voltage was below 1000 and did not interfere with the measurements.

Dot blot. Different amounts (0-400 ng) of human MIF, mouse MIF, or human CXCL12 were spotted on a nitrocellulose membrane and membranes allowed to dry for 30 min. Non-specific binding was blocked with Tris-buffered saline (TBS), pH 7.4, containing 0.1% Tween-20 (TBS-T) and 1% BSA. TAMRA-msR4M-L1 was reconstituted at a concentration of 10 μM in PBS containing 2.5% HFIP, diluted to a 3 μM working solution in 1% BSA/TBS-T, and incubated with the membrane at 4° C. Fluorescence intensities were measured at 600 nm using an Odyssey® Fc imager (LICOR Biosciences, Bad Homburg, Germany). The total intensity of each spot was automatically corrected by the individual background signal. The signal intensity of 400 ng human MIF was set to 100%.

Microscale thermophoresis. MST measurements were recorded on a Monolith NT.115 instrument with green/red filters (NanoTemper Technologies, Munich, Germany). MST power was set at 80% and LED power was at 95%: all measurements were performed at 37° C. MST traces were tracked for 40 s (laser-off: 5 s, laser-on: 30 s; laser-off: 5 s). A stock solution of 200 nM TAMRA-msR4M-L1 was prepared in 20 mM sodium phosphate buffer, pH 7.2, containing 0.2% Tween-20. For titration of MIF, sub-stock solutions were prepared by serial 1:1 dilutions from a 20 μM stock solution in 20 mM sodium phosphate buffer, pH 7.2. TAMRA-msR4M-L1 and each MIF sub-stock were mixed at a 1:1 ratio, incubated for 10 min and loaded in the capillaries. Experimental measurement values by the temperature jump (T-Jump) setting.

The setup was similar for the titrations between MST-Red-MIF and soluble human CD74 (sCD74). Soluble CD74 has been described¹⁹ and is a fusion protein of an N-terminal HA-tag and CD74 residues 73-232 (R&D Systems, Minnesota, USA). The stock solution of sCD74 (4 μM) was prepared in PBS (1×, pH 7.2) and MST-Red-MIF dissolved at a concentration of 100 nM in PBS containing 0.01% BSA. Sub-stocks of sCD74 for titration were prepared by serial 1:1 dilution in 1×PBS, pH 7.2, containing 0.005% BSA. To test if MIF/sCD74 binding is affected by msR4M-L1, MST-Red-MIF (100 nM) was pre-mixed with msR4M-L1 (4 μM) and sCD74 titrations performed as above. App. K_{D} values were calculated assuming a 1/1 binding model.

CXCR4-specific signaling in a yeast-based cell system. The yeast CXCR4-specific cell signaling system employing S. cerevisiae strain (CY12946), expressing functional CXCR4 that replaces the yeast STE2 receptor and is linked to a β-galactosidase (lacZ) signaling read-out, has been described^{24, 25}. CXCL12 and MIF elicit a CXCR4-specific signaling response in this cell system^{25, 26}. Briefly, yeast transformants stably expressing human CXCR4 were grown overnight at 30° C. in yeast nitrogen base selective medium (Formedium, UK). Cells were diluted to an OD₆₀₀ of 0.2 and grown to an OD₆₀₀ of 0.3-0.6. Transformants were incubated with 20 μM human MIF or 2 μM human CXCL12 in the presence or absence of different concentrations of msR4M-L1 for 1.5 h. OD₆₀₀ was measured and activation of CXCR4 signaling quantified by β-galactosidase activity using a commercial BetaGlo Kit (Promega, Mannheim, Germany).

Cell culture and cell lines. Human aortic endothekal cells (HAoECs) were from PromoCel (Heidelberg, Germany). Cells were plated on collagen (Biochrom AG, Berlin, Germany) in endothelial cell growth medium (ECGM, PromoCell) and cultured as described⁶⁸. The monocytic cell line MonoMac-6 was cultured in RPMI 1640 medium with 10% fetal calf serum (FCS) as established¹⁴. Primary human cardiac myocytes (HCM) isolated from the ventricles of the adult heart were from PromoCell and used at passage 2-8. They were cultured in myocyte basal medium (PromoCell), containing 5 μg/mL insulin, 5% FCS, 2 ng/mL fibroblast growth factor (FGF), and 0.5 ng/mL epidermal growth factor (EGF). Human embryonic kidney (HEK)-293 cells were cultured in DMEM-GlutaMAX (Life Technologies-Gibco) supplemented with 10% FCS and 1% penicillin/streptomycin. FCS was obtained from Invitrogen-Thermo Fisher Scientific. Miscellaneous cell culture reagents (media, supplements) were bought from Invitrogen and PAA (Pasching, Austria).

HEK293-CD74 surface binding assay. HEK293 cells were transiently transfected with 8 μg of the pcDNA3.1-CD74minRTS-FLAG plasmid using Polyfect (Qiagen. Hilden. Germany) and expressed surface CD74 after 24 h (efficiency 50-80%), as described⁶⁹. HEK293-CD74 transfectants were washed and 3×10⁵ cells resuspended in ice-cold PBS containing 0.5% BSA, and incubated with 400 nM Alexa-488-labeled MIF in the presence or absence of msR4M-L1 (2 μM) on ice for 2 h. After washing in ice-cold PBS containing 0.1% BSA, the amount of Alexa-488-labeled MIF bound to the cell surface was quantified by flow cytometry using a FACS Verse instrument (BD Biosciences, Heidelberg, Germany). Binding of Alexa-488-MIF to non-transfected “wildtype” HEK293 cells, which do not express CD74, served as background control.

Mice. Mice were housed under standardized light-dark cycles in a temperature-controlled air-conditioned environment under specific pathogen-free conditions at the Center for Stroke and Dementia Research (CSD). Munich. Germany, with free access to food and water. AN mice used in this study were between 7-10 weeks or age and were on C57BL/6 background. Apoe^{-/-} mice were initially obtained from Charles River Laboratories (Sulzfeld, Germany) and backcrossed within the CSD animal facility before use. The atherogenic Ldlr^{-/-} and Ldlr^{-/-} Mif^{-/-} mice as well as Apoe^{-/-} Mif^{-/-} mice have been described previously^{14, 61}. All mouse experiments were approved by the Animal Care and Use Committee of the local authorities and performed in accord with the animal protection representative at CSD.

Chemotaxis analysis of murine B cells. A Transwell-based assay was used as described previously³⁴. Briefly, splenic B cells were isolated by negative depletion using a Pan B Cell Isolation Kit (Miltenyi Biotec. Bergisch Gladbach, Germany). Purity of the cells was between 95 and 99%. One-hundred μL of cell suspension containing 1×10⁶ cells in RPMI 1640/5% FCS was loaded into the upper chamber of a Transwell insert. Filters were transferred into the lower chambers containing MIF or CXCL12 in the presence or absence of ectodomain peptides. Chemotaxis was followed for 4 h at 37° C. in a humidified atmosphere of 5% CO₂. Migrated cells were counted by flow cytometry using CountBright™ Absolute Counting Beads (Molecular Probes-Invitrogen).

CD74 signaling in human cardiomyocytes. Before stimulation, medium was replaced by fresh myocyte basal medium containing 0.05% FCS and HCMs rested for 16 h. Surface CD74 expression on HCMs was verified by flow cytometry (FITC-conjugated anti-human CD74, FITC-IgG2 (isotype control) (BD Pharmingen), 1 h/4° C. in the dark, BD FACSVerse™ flow cytometer, FlowJo software). AMPK signaling was elicited by addition of human MIF (16 nM, 60 min) following an established procedure¹⁵. To test for an influence of msR4M-L1, MIF was preincubated with 16 or 80 nM msR4M-L1 and mixtures added to HCMs. After treatment, cells were lysed and subjected to SDS-PAGE/Western blotting. AMPK activation was revealed with an antibody against phosphorylated AMPK (anti-pAMPKα, 1:1000, Cell Signaling Technologies, Heidelberg, Germany) and total AMPKα (anti-AMPKα, 1:1000), as well as actin detected for standardization. Anti-rabbit horseradish peroxidase (HRP)-conjugated antibody (1:10000, GE Healthcare, Freiburg, Germany) was used for development and signals quantitated by chemiluminescence using an Odyssey® Fc imager.

Isolation of human peripheral blood-derived monocytes. Human peripheral blood-derived monocytes were isolated as described¹⁴. Briefly, blood was collected from healthy donors or buffy coat obtained from the blood bank of Munich University Hospital, mixed 1:1 with PBS, and PBMCs isolated by Ficoll-Paque Plus gradient (GE Healthcare). Monocytes were purified by negative depletion using the Monocyte Isolation Kit II (Miltenyi). Monocyte purity was verified by flow cytometry using an anti-CD14 antibody (Miltenyl) and was 95-98%. Purified cells were suspended in RPMI 1640 medium supplemented with 10% FCS, 1% penicillin/streptomycin, 2 mM L-glutamine and 1% NEAA. The isolation of PBMCs from donor blood was approved by the local ethics committee of LMU Munich. 3D migration of human peripheral blood-derived monocytes by time-lapse microscopy. The 3D-migration behavior of human monocytes was assessed by time-lapse microscopy and individual cell tracking using the 3D chemotaxis μ-Slide system from Ibidi GmbH (Munich, Germany), adapting the established Ibidi dendritic cell protocol for human monocytes. Briefly, isolated monocytes (4×10⁶ cells) were seeded in rat tail collagen type-I gel in DMEM and subjected to a gradient or MIF or CXCL12 (64 nM) in the presence or absence of msR4M-L1. Cell motility was monitored performing time-lapse imaging every 1 min at 37° C. for 2 h using a Leica inverted DMi8-Life Cell Imaging System equipped with a DMC2900 Digital Microscope Camera with CMOS sensor and live cell-imaging software (Leica Microsystems, Wetzlar, Germany). Images were imported as stacks to ImageJ software and analyzed with the manual tracking and chemotaxis/migration tools (Ibidi GmbH).

Dil-LDL uptake/foam cell formation. MIF/CXCR4-dependent foam cell formation was assessed by measuring uptake of fluorescently labeled human low density lipoprotein particles (Dil-LDL) in primary human monocyte-derived macrophages following a described protocol³⁶. Briefly, cells were incubated in culture medium (RPMI 1640-GlutaMAx medium containing 100 U/mL penicillin, 100 μg/mL streptomycin, and 0.2% BSA) for 15 h at 37° C. and subsequently incubated in the same medium supplemented with 1% HPCD ((2-hydroxy)-β-cyclodextrin, Sigma-Aldrich) for 45 min. After washing with imaging solution (MEM without phenol red containing 30 mM HEPES, 0.5 g/L NaHCO₃, pH 7.4, and 0.2% BSA), cells were exposed to 50 μg/mL 1,1′-dioctadecyl-3,3,3′3′-tetra-methylindocarbocyanine-labeled LDL (Dil-LDL) for 30 min at 4° C. followed by incubation at 37° C. for 20 min. Cells were washed with ice-cold imaging solution (pH 3.5), fixed, and counter-stained with Hoechst 33258.

Static monocyte adhesion. HAoECs were seeded at a density of 30.000 cells/well in 6 well μ-Ibidi Perfusion slides VI 0.4 (Ibidi GmbH). After overnight incubation, human TNF-α or MIF were added at a final concentration of 4 or 16 nM, respectively, in the presence versus absence of msR4M-L1 (320 nM), and cells incubated for 16 h. After perfusion of the chambers with fresh medium, MonoMac6 cells (1×10⁶ cells/mL) in PromoCell medium were added for 30 min. Non-adhering cells were flushed away by gentle perfusion using a 30 mL syringe. To quantify adherent monocytes, 10 individual images from each treatment were acquired using a Leica DMi8 inverted microscope with a 10× objective and cells quantified using Image J.

Staining of atherosclerotic plaque tissue with Fluos-msR4M-L1. Immunofluorescent staining of atherosclerotic tissue with Fluos-msR4M-L1 was performed with specimens from atherogenic Ldlr^{-/-} and Apoe^{-/-} mice. Ldlr^{-/-} mice were on chow diet for 30 weeks and developed native atherosclerotic lesions as reported previously¹⁴. Mif-deficient mice (Ldlr^{-/-} Mif^{-/-}) were used for comparison. Aortic root sections were deparaffinized and rehydrated. For antigen retrieval, slides were boiled in sodium citrate buffer, pH 6.0, 0.05% Tween-20, and blocked with PBS, containing 5% donkey serum and 1% BSA. For staining, slides were incubated at 4° C. with Fluos-msR4M-L1 (5 μM) in blocking buffer. DAPI was used for nuclear counterstain and sections were imaged using a Leica DMI8 fluorescent microscope. The mean fluorescence intensity localized to the aortic vessel wall was quantified via Image J.

For Apoe^{-/-} mice (and Apoe^{-/-} Mif^{-/-} as control⁶¹), cryo-conserved sections of advanced lesions from brachiocephalic artery (BC) were used from mice on Western-type high-rat diet (HFD, 1.25% cholesterol) for 24 weeks. Sides were fixed in ice-cold acetone, rehydrated in PBS, and blocked in PBS/1% BSA, incubated with 500 nM Fluos-msR4M-L1, and analyzed as above.

Fluos-msR4M-L1 staining and monocyte adhesion in atherosclerotic carotid arteries by multiphoton microscopy. Monocyte adhesion experiments in atherosclerotic carotid arteries under physiological flow conditions ex vivo have been established^{14, 70}. Seven-week-old Apoe^{-/-} mice were fed a Western-type HFD (0.2% cholesterol) for 12 weeks. The last three days before sacrifice, mice were injected with msR4M-L1 (100 μg, once daily) or sterile saline (control). On day 3, arteries were prepared and mounted into an arteriograph chamber as described⁷⁰. Carotids were flushed with buffer containing msR4M-L1 (3 μM). Mouse leukocytes isolated from the bone marrow of msR4M-L1- or vehicle-treated atherogenic Apoe^{-/-} mice were stained with fluorescent Green CMFDA or Red CMPTX (Thermo Fisher Scientific). After washing with Hank's Balanced Salt Solution (HBSS), stained leukocytes were incubated with 3 μM msR4M-L1 (red) or PBS (green, control) for 1 h at 37° C. The red- and green-stained cell pools were mixed at a 1:1 ratio and 3×10⁶ cells in 6 mL perfused into the artery of msR4M-L1- or vehicle-treated mice, respectively. Arteries were scanned by MPM using a multispectral TCS SP8 DIVE instrument with filter-free 4TUNE NDD detection module (Leica) and the number of adherent and transmigrated leukocytes determined by scanning multi-photon excitation. Vessel structure (and plaques) were visualized by second harmonic generation (SHG).

For plaque staining with Fluos-msR4M-L1 in carotid arteries ex vivo, Fluos-msR4M-L1 was i.p.-injected into aged atherogenic Apoe^{-/-} mice (24-week HFD) three days before carotid preparation (50 μg per, once daily), arteries prepared and staining inspected by MPM as above.

Proteolytic stability assay. Human plasma was prepared from blood of healthy volunteers by standard procedure. Biotin-6-Ahx-msR4M-L1 was dissolved in PBS and mixed with PBS or human plasma (final concentration 70 μM) and solutions incubated for 0.5, 1, 4, or 16 h at 4° C. or 37′C. Samples were then diluted in 2× Novex Tricine SDS sample buffer (Life Technologies) at a ratio of 1:6. Samples were electrophoresed in a 10-20% Tricine gel, transferred to nitrocellulose, and biotin-6-Ahx-msR4M-L1 revealed by streptavidin-POD conjugate (Roche Diagnostics, Mannheim. Germany; 1:5000 dilution), using an Odyssey Fc imager.

Cytokine array. Cytokine/chemokine profiling was performed from plasma samples of msR4M-L1-versus vehicle-treated Apoe^{-/-} mice using mouse cytokine array panel A (R&D Systems, ARY006) according to the manufacturer's instructions. Plasma samples were diluted (1:10) in array buffer; incubated with antibody detection cocktail for 1 h at RT, exposed to the blocked membranes (overnight, 4° C.), membranes washed and incubated with streptavidin-HRP conjugate working solution (30 min, RT). Membranes were developed with Chemi-Reagent Mix and analyzed by Odyssey® Fc imager. The average signal (pixel density) of duplicate spots was quantified by ImageJ.

In-Vivo Model of Atherosclerosis

Therapeutic injections of msR4M-L1 and aorta preparation. Seven-eight-week-old female Apoe^{-/-} mice were randomly divided into two groups of 11-12 mice each and both groups put on a Western-type HFD (0.2% cholesterol) for 4.5 weeks. Mice develop early-to-intermediate atherosclerotic lesions in this model³⁹. One group was i.p.-injected with 50 μg msR4M-L1 dissolved in saline every other day for 4.5 weeks; controls received saline. No toxicity or side effects were noted. At the end of the experiment, mice were sacrificed, blood collected by cardiac puncture and saved for blood cell and lipid measurements and mice transcardially perfused with saline. Hearts, proximal aortas and carotid arteries were prepared and fixed for plaque morphometry and lesion analysis.

Quantification of plaques and vessel morphometry (oil red O and H&E staining). Cut heart tissue containing aortic root were embedded in optimum cutting temperature (OCT) (Sakura Finetek, Osaka, Japan) and frozen at −80° C. Eight-μm sections were prepared for oil-red O (ORO) staining and plaque immune cell analysis. The accumulation of macrophages in aortic root lesions was determined by an anti-MAC-2 antibody followed by Cy5-conjugated secondary antibody. Nuclei were visualized with DAPI. The aortic arch was cut, fixed in 4% paraformaldehyde (PFA) and embedded in paraffin. Ten-μm sections containing the three branches (brachiocephalic, left common carotid, and left subclavian artery) were prepared and stained with hematoxylin/eosin (HE) for vessel morphometry. Images were captured with a Leica DMi8 microscope and quantified using Image J.

Blood cell counts, triglycerides and cholesterol levels. Blood was collected in EDTA tubes and leukocytes and plasma obtained by centrifugation at 630×g (10 min, 4° C.). For leukocyte counts, red blood cells (RBC) were depleted by RBC-lysis buffer (BioLegend) at RT, leukocytes washed and suspended in PBS containing 0.5% BSA. Cells were stained with an antibody cocktail comprising APC-Cy-7-conjugated anti-CD45, PE-conjugated anti-CD11b. APC-conjugated anti-CD19, FITC-conjugated anti-CD3. APC-conjugated anti-Ly6C. and PE-conjugated anti-Ly6G (BD Biosciences). Measurements were analyzed using a BD FACSVerse™ flow cytometer and data quantified using FlowJo software.

Total cholesterol and triglyceride concentrations were measured enzymatically using routine cholesterol fluorometric and triglyceride colorimetric assay kits, respectively (Cayman Chemical Company. Ann Arbor. USA).

Analysis of Human Carotid Atherosclerotic Plaques

Patient population, study groups and tissue samples. Carotid artery tissue samples (n=28) came from the Munich Vascular Biobank (MVB) and were from patients who underwent carotid endarterectomy (CEA) in the Department of Vascular and Endovascular Surgery at University Hospital of Technische Universität München. Preparation of samples for histological and IHC analysis has been reported⁴⁰. Carotid specimens were fixed in formalin and embedded in paraffin (FFPE) and used to evaluate the expression of MIF by antibody or for staining with Fluos-msR4M-L1. Healthy FFPE carotid vessels were obtained from the Forensic Medicine Department (n=6). The type of atherosclerotic lesions in the CEA samples was determined according to the American Heart Association (AHA) guidelines using HE and Elastica-van-Gieson (EVG) staining procedures as described⁴⁰. All carotid tissues used showed advanced atherosclerosis (stage V-VII). The study was approved by the local ethical committee of the University Hospital and followed the Guidelines of the World Medical Association Declaration of Helsinki. All patients provided informed consent. Immunohistochemistry/immunofluorescence staining. Immunofluorescence staining of human CEA tissues with Fluos-msR4M-L1 was performed using the same protocol as for paraffin-embedded specimens from Ldlr^{-/-} mice (see above). Antibody-based detection of MIF was performed applying the DAB+ kit (Abcam, ab64238) following the standard protocol. MIF was detected with the polyclonal goat antibody N-20 (Santa Cruz, sc-16965; 1:100). HRP-conjugated polyclonal rabbit anti-goat immunoglobulin (DAKO, P0160, 1:1000) was used as secondary antibody. Slides were counterstained with Mayer hematoxylin and stainings analyzed with a Leica DMi8.

Statistical Analysis. Statistical analysis was performed using GraphPad Prism version 7 and 8 software. Data are represented as means±SD. After testing for normality, data were analyzed by two-tailed Student's t-test, Mann-Whitney U, or Kruskal-Wallis test as appropriate. Differences with p<0.05 were considered to be statistically significant.

### REFERENCES

1. Charo. I. F. & Ransohoff, R. M. The many roles of chemokines and chemokine receptors in inflammation. N Eng J Med 354, 610-621 (2006).
2. Bachelerie, F. et al. International Union of Basic and Clinical Pharmacology. LXXXIX. Update on the extended family of chemokine receptors and introducing a new nomenclature for atypical chemokine receptors. Pharmacol Rev 66, 1-79 (2014).
3. Koenen, R. R. & Weber, C. Chemokines: established and novel targets in atherosclerosis. EMBO Mot Med 3, 713-725 (2011).
4. Ridker, P. M. et al. Antiinflammatory therapy with Canakinumab for atherosclerotic disease. N Eng J Med 377, 1119-1131 (2017).
5. Proudfoot, A. E., Bonvin, P. & Power, C. A. Targeting chemokines: Pathogens can, why can't we? Cytokine 74, 259-267 (2015).
6. Weber, C. & Noels, H. Atherosclerosis: current pathogenesis and therapeutic options. Nat Med 17, 1410-1422 (2011).
7. Femandez-Botran, R., Crespo, F. A. & Sun, X. Soluble cytokine receptors in biological therapy. Expert Opin Biol Ther 2, 565-605 (2002).
8. Calandra, T. & Roger. T. Macrophage migration inhibitory factor: a regulator of innate immunity. Nat Rev Immunol 3, 791-800 (2003).
9. Bernhagen, J. et al. MIF is a pituitary-derived cytokine that potentiates lethal endotoxaemia. Nature 365, 756-759 (1993).
10. David. J. R. Delayed hypersensitivity in vitro: its mediation by cell-free substances formed by lymphoid cell-antigen interaction. Proc Natl Acad Sci USA 56, 72-77 (1966).
11. Donnelly, S. C. et al. Regulatory role for macrophage migration inhibitory factor in acute respiratory distress syndrome. Nat. Med. 3, 320-323 (1997).
12. Morand, E. F., Leech, M. & Bernhagen, J. MIF: a new cytokine link between rheumatoid arthritis and atherosclerosis. Nat Rev Drug Discov 6, 399-410 (2006).
13. Merk, M. et al. The D-dopachrome tautomerase (DDT) gene product is a cytokine and functional homolog of macrophage migration inhibitory factor (MIF). Proc Natl Acad Sci USA 108, E577-585 (2011).
14. Bernhagen, J. et al. MIF is a noncognate ligand of CXC chemokine receptors in inflammatory and atherogenic cell recruitment. Nat Med 13, 587-596 (2007).
15. Miller, E. J. et al. Macrophage migration inhibitory factor stimulates AMP-activated protein kinase in the ischaemic heart. Nature 451, 578-582 (2008).
16. Sinitski, D. et al. Macrophage migration inhibitory factor (MIF)-based therapeutic concepts in atherosclerosis and inflammation. Thromb Haemost 119, 553-566 (2019).
17. Zemecke, A., Bernhagen, J. & Weber, C. Macrophage migration inhibitory factor in cardiovascular disease. Circulation 117, 1594-1602 (2008).
18. Kleemann, R. et al. Intracellular action of the cytokine MIF to modulate AP-1 activity and the cell cycle through Jab1. Nature 408, 211-216 (2000).
19. Leng, L. et al. MIF signal transduction initiated by binding to CD74. J Exp Med 197, 1467-1476 (2003).
20. Qi, D. et al. The vestigial enzyme D-dopachrome tautomerase protects the heart against ischemic injury. J Clin Invest 124, 3540-3550 (2014).
21. Assis, D. N. et at. The role of macrophage migration inhibitory factor in autoimmune liver disease. Hepatology 59, 580-591 (2014).
22. Pantouris, G. et al. Nanosecond dynamics regulate the MIF-induced activity of CD74. Angew Chem Int Ed Engl 67, 7116-7119 (2018).
23. Weber, C. et al. Structural determinants of MIF functions in CXCR2-mediated inflammatory and atherogenic leukocyte recruitment. Proc Natl Acad Sci USA 105, 16278-16283 (2008).
24. Lacy. M. et al. Identification of an Arg-Leu-Arg tripeptide that contributes to the binding interface between the cytokine MIF and the chemokine receptor CXCR4. Sci Rep 8, 5171 (2018).
25. Rajasekaran, D. et al. Macrophage migration inhibitory factor-CXCR4 receptor interactions: evidence for partial allosteric agonism in comparison with CXCL12 chemokine. J Biol Chem 291, 15881-15895 (2016).
26. Doring. Y. et al. Vascular CXCR4 limits atherosclerosis by maintaining arterial integrity: evidence from mouse and human studies. Circulation 136, 388-403 (2017).
27. Zemecke, A. et al. Delivery of microRNA-126 by apoptotic bodies induces CXCL12-dependent vascular protection. Sci Signal 2, ra81 (2009).
28. Spanopoulou, A. et al. Designed macrocyclic peptides as nanomolar amyloid inhibitors based on minimal recognition elements. Angew Chem Int Ed Engl 57, 14503-14508 (2018).
29. VW, B. et al. Structures of the CXCR4 chemokine GPCR with small-molecule and cyclic peptide antagonists. Science 330, 1066-1071 (2010).
30. Qin, L. et al. Structural biology. Crystal structure of the chemokine receptor CXCR4 in complex with a viral chemokine. Science 347, 1117-1122 (2015).
31. Crump, M. P. et al. Solution structure and basis for functional activity of stromal cell-derived factor-1; dissociation of CXCR4 activation from binding and inhibition of HIV-1. EMBO J 16, 6996-7007 (1997).
32. Yan, L. M., Tatarek-Nossol, M., Velkova. A., Kazantzis, A. & Kapumiotu, A. Design of a mimic of nonamyloidogenic and bioactive human islet amyloid polypeptide (IAPP) as nanomolar affinity inhibitor of IAPP cytotoxic fibrillogenesis. Proc Natl Acad Sci USA 103, 2046-2051 (2006).
33. Eftink, M. R. Fluorescence methods for studying equilibrium macromolecule-ligand interactions. Methods Enzymol 278, 221-257 (1997).
34. Klasen, C. et al. MIF promotes B cell chemotaxis through the receptors CXCR4 and CD74 and ZAP-70 signaling. J Immunol 192, 5273-5284 (2014).
35. Bernhagen, J. et al. Purification, bioactivity, and secondary structure analysis of mouse and human macrophage migration Inhibitory factor (MIF). Biochemistry 33, 14144-14155 (1994).
36. Domschke, G. et al. Systematic RNA-interference in primary human monocyte-derived macrophages: A high-throughput platform to study foam cell formation. Sci Rep 8, 10516 (2018).
37. Kerschbaumer. R. J. et al. Neutralization of macrophage migration inhibitory factor (MIF) by fully human antibodies correlates with their specificity for the p-sheet structure of MIF. J Biol Chem 287, 7446-7455 (2012).
38. Leng, L. et al. A small-molecule macrophage migration inhibitory factor antagonist protects against glomerulonephritis in lupus-prone NZB/NZW F1 and MRL/lpr mice. J Immunol 186, 527-538 (2011).
39. Rami, M. et al. Chronic intake of the selective serotonin reuptake inhibitor fluoxetine enhances atherosclerosis. Arterioscler Thromb Vasc Bio 38, 1007-1019 (2018).
40. Merckelbach, S. et al. Expression and cellular localization of CXCR4 and CXCL12 in human carotid atherosclerotic plaques. Thromb Haemost 118, 195-206 (2018).
41. Rose-John, S. The soluble interleukin 6 receptor advanced therapeutic options in inflammation. Clin Pharmacol Ther 102, 591-598 (2017).
42. Zlotnik, A., Burkhardt, A. M. & Homey. B. Homeostatic chemokine receptors and organ-specific metastasis. Nat Rev Immunol 11. 597-606 (2011).
43. Pawig. L., Klasen, C., Weber, C., Bernhagen, J. & Noels, H. Diversity and inter-connections in the CXCR4 chemokine receptor/ligand family: molecular perspectives. Front Immunol 6, 429 (2015).
44. DiPersio, J. F., Uy, G. L., Yasothan, U. & Kirkpatrick, P. Plerixafor. Nat Rev Drug Discov 8, 105-106(2009).
45. Schunkert, H. et al. Large-scale association analysis identifies 13 new susceptibility loci for coronary artery disease. Nat Genet 43, 333-338 (2011).
48. Doring. Y. et al. CXCL12 derived from endothelial cells promotes atherosclerosis to drive coronary artery disease. Circulation 139, 1338-1340 (2019).
47. Sjaarda, J. et al. Blood CSF1 and CXCL12 as causal mediators of coronary artery disease. J Am Coll Cardiol 72, 300-310 (2018).
48. Liehn, E. A. et al. Double-edged role of the CXCL12/CXCR4 axis in experimental myocardial infarction. J Am Coll Cardiol 68, 2415-2423 (2011).
49. Zemecke, A. et al. SDF-1alpha/CXCR4 axis is instrumental in neointimal hyperplasia and recruitment of smooth muscle progenitor cells. Circ Res 96, 784-791 (2005).
50. Hu, X. et al. Stromal cell derived factor-1 alpha confers protection against myocardial ischemia/reperfusion injury: role of the cardiac stromal cell derived factor-1 alpha CXCR4 axis. Circulation 116, 654-663 (2007).
51. Saxena, A. et al. Stromal cell-derived factor-1alpha is cardioprotective after myocardial infarction. Circulation 117, 2224-2231 (2008).
52. Zemecke, A. et al. Protective role of CXC receptor 4/CXC ligand 12 unveils the importance of neutrophils in atherosclerosis. Circ Res 102, 209-217 (2008).
53. Fingerle-Rowson, G. et al. A tautomerase-null macrophage migration-inhibitory factor (MIF) gene knock-in mouse model reveals that protein interactions and not enzymatic activity mediate MIF-dependent growth regulation. Mol Cell Biol 29, 1922-1932 (2009).
54. Barter, E. F. & Stone, M. J. Synergistic interactions between chemokine receptor elements in recognition of interleukin-8 by soluble receptor mimics. Biochemistry 51, 1322-1331 (2012).
55. Mobius. K., Durr, R., Haussner, C., Dietrich, U. & Eichler, J. A functionally selective synthetic mimic of the HIV-1 co-receptor CXCR4. Chemistry 18, 8292-8295 (2012).
56. Pritz, S. et al. Synthesis of protein mimics with nonlinear backbone topology by a combined recombinant, enzymatic, and chemical synthesis strategy. Angew Chem Int Ed Engl 47, 3642-3645 (2008).
57. Mahalingam, D. et al. Safety and efficacy analysis of imalumab, an anti-oxidized macrophage migration inhibitory factor (oxMIF) antibody, alone or in combination with 5-fluorouracil/leucovorin (5-FU/LV) or panikumumab, in patients with metastatic colorectal cancer (mCRC). Ann Oncol; 2016: 2016. p. ii105.
58. Ziarek, J. J. et al. Structural basis for chemokine recognition by a G protein-coupled receptor and implications for receptor activation. Sci Signal 10 (2017).
59. Chen, D. et al. Expression of human tissue factor pathway inhibitor on vascular smooth muscle cells inhibits secretion of macrophage migration inhibitory factor and attenuates atherosclerosis in ApoE−/− mice. Circulation 131, 1350-1360 (2015).
60. Burger-Kentischer, A. et al. Expression of macrophage migration inhibitory factor in different stages of human atherosclerosis. Circulation 105, 1561-1566 (2002).
61. Schmitz, C. et al. Mif-deficiency favors an atheroprotective autoantibody phenotype in atherosclerosis. FASEB J 32, 4428-4443 (2018).
62. von Hundelshausen, P. et al. Chemokine interactome mapping enables tailored intervention in acute and chronic inflammation. Sci Trans Med 9 (2017).
63. Kang, I. & Bucala, R. The immunobiology of MIF: function, genetics and prospects for precision medicine. Nat Rev Rheumatol 15, 427-437 (2019).
64. Dessein, A. F. et al. Autocrine induction of invasive and metastatic phenotypes by the MIF-CXCR4 axis in drug-resistant human colon cancer cells. Cancer Res 70, 4644-4654 (2010).
65. Lutgens, E. et al. Immunotherapy for cardiovascular disease. Eur Heart J epub-ahead of print (2019).
66. Henninot, A., Collins. J. C. & Nuss. J. M. The current state of peptide drug discovery: back to the future? J Med Chem 61, 1382-1414 (2018).
67. Andreetto, E. et al. A hot-segment-based approach for the design of cross-amyloid interaction surface mimics as inhibitors of amyloid self-assembly. Angew Chem Int Ed 54. 13095-13100 (2015).
68. Asare. Y. et al. Inhibition of atherogenesis by the COP9 signalosome subunit 5 in vivo. Proc Nat Acad Sci USA 114, E2766-E2775 (2017).
69. Yoo, S. A. et al. MIF allele-dependent regulation of the MIF coreceptor CD44 and role in rheumatoid arthritis. Proc Natl Acad Sci USA 113, E7917-E7926 (2016).
70. van der Vorst, E. P. C. et al. Functional ex-vivo imaging of arterial cellular recruitment and lipid extravasation. Bio Protoc 7 (2017).

1. A chemokine-selective CXCR4 ectodomain-derived (poly)peptide comprising or consisting of a first peptide of (X1)(X2)(X3)(X4)(X5)WYFGNF(X6)(X7)(X8) (SEQ ID NO: 1) linked via a linker to a second peptide of (Y1)(Y2)(Y3)(Y4)(Y5)D(Y6)FY(Y7)N(Y8)LW(Y9) (SEQ ID NO: 2), wherein
(X1) is present or absent and, if present, is an amino acid, preferably D or A
(X2) is present or absent and, if present, is an amino acid, preferably A or G
(X3) is present or absent and, if present, is an amino acid, preferably V or A
(X4) is present or absent and, if present, is an amino acid, preferably A or G
(X5) is present or absent and, if present, is an amino acid, preferably N
(X6) is an amino acid, preferably L or A
(X7) is an amino acid, preferably C, A or S, more preferably C or A
(X8) is an amino acid, preferably K or A
(Y1) is present or absent and, if present, is an amino acid, preferably D or A
(Y2) is present or absent and, if present, is an amino acid, preferably R or A
(Y3) is present or absent and, if present, is an amino acid, preferably Y
(Y4) is present or absent and, if present, is an amino acid, preferably I or A
(Y5) is present or absent and, if present, is an amino acid, preferably C, A or S, more preferably C or A
(Y6) is present or absent and, if present, is an amino acid, preferably D, R or A
(Y7) is an amino acid, preferably P or A
(Y8) is an amino acid, preferably D or A
(Y9) is present or absent and, if present, is an amino acid, preferably V
and wherein said linker has a length of 0.2 to 5 nm, preferably 1 nm to 5 nm, more preferably 2 to 4 nm, and most preferably about 2.358 nm.

2. The (poly)peptide of claim 1, wherein the linker comprises or consists of 1 to 8, and preferably 2 or 3 amino acids.

3. The (poly)peptide of claim 1, wherein the linker comprises or consists of non-natural amino acids.

4. The (poly)peptide of claim 3, wherein the non-natural amino acids are selected from the group consisting of 6-aminohexanoic acid (6-Ahx), 12-amino-dodecanoic acid (12-Ado) and 3,6-dioxaoctanoic acid (O20c).

5. The (poly)peptide of claim 1, wherein the linker comprises or consists of 6-Ahx-12-Ado or O2Oc-12-Ado, and preferably consists of 6-Ahx-12-Ado.

6. The (poly)peptide of claim 1, wherein the linker comprises or consists of three naturally-occurring amino acids, preferably selected from G, D, R and K, wherein the linker is most preferably selected from DDD and RRR.

7. The (poly)peptide of claim 1, wherein the (poly)peptide is a cyclic (poly)peptide.

8. The (poly)peptide of claim 7, wherein the (poly)peptide comprises two cysteines or homocysteines being linked by an S—S bond.

9. The (poly)peptide of claim 8, wherein the two cysteines or homocysteines are preferably located at an N-terminus of the first peptide and a C-terminus of the second peptide.

10. The (poly)peptide of claim 1, wherein the (poly)peptide is fused to
(i) a component modulating serum half-life, wherein the component modulating serum half-life is preferably Fc domain of an antibody, an albumin binding tag, albumin or polyethylene glycol,
(ii) a component increasing solubility of the (poly)peptide, wherein the component increasing the solubility of the (poly)peptide is preferably selected from a peptide comprising acids with positively and negatively charged side chains, betaines, polyionic tags, cyclodextrins, glycosyl moieties, and conjugated nanoparticles, and/or
(iii) a diagnostic label, preferably a chromogenic label, a fluorogenic label, or an isotope.

11. The (poly)peptide of claim 1, wherein the (poly)peptide comprises a first peptide differing by no more than three, preferably by no more than two, more preferably by one amino acid mutation and most preferably by zero amino acid mutations from the peptide of DAVANWYFGNFLCK (SEQ ID NO: 3) and/or comprises a second peptide differing by no more than three, preferably by no more than two, more preferably by one amino acid mutation and most preferably by zero amino acid mutations from the peptide of DRYICDRFYPNDLWV (SEQ ID NO: 4).

12. A composition, preferably a pharmaceutical composition comprising the (poly)peptide of claim 1.

13-15. (canceled)

16. A method of treating a disease, comprising using the (poly)peptide of claim 1.

17. The method of claim 16, wherein the disease is an atherosclerotic disease, an inflammatory disease, a tumor, a neuroinflammatory or neuro-degenerative disease, or an autoimmune disease.

18. The method of claim 17, wherein
the atherosclerotic disease is an atherosclerotic disease in individuals with a high-MIF expression genotype as defined by the CATT6-8 or CATT-non-5/5 promoter polymorphism; and/or
wherein the tumor is cancer, preferably metastatic cancer.
